(19) United States
(12) Patent Application Publication
(10) Pub. No.: US 2025/0320215 A1
(43) Pub. Date: Oct. 16, 2025

(54) SALTS OF N-[4-(4-[[2-(DIMETHYLAMINO)ETHYL]AMINO]-3-METHYL-1H-PYRAZOLO[3,4-D]PYRIMIDIN-6-YL)-2-FLUOROPHENYL]-2,5-DIFLUOROBENZENESULFONAMIDE AND CRYSTALLINE FORMS THEREOF

(71) Applicant: THRYV THERAPEUTICS INC, Montreal (CA)

(72) Inventors: Marc VIDAL, Montreal (CA); Maroua KHALIFA, Montreal (CA); Liyu WANG, Beijing (CN); Wenying DAI, Beijing (CN)

(73) Assignee: THRYV THERAPEUTICS INC, Montreal, QU (CA)

(21) Appl. No.: 19/017,207

(22) Filed: Jan. 10, 2025

Related U.S. Application Data
(63) Continuation of application No. PCT/CA2023/050930, filed on Jul. 11, 2023.
(60) Provisional application No. 63/368,208, filed on Jul. 12, 2022.

(51) Int. Cl. C07D 487/04; A61K 31/519

(52) U.S. Cl. CPC C07D 487/04; A61K 31/519

(57) ABSTRACT

Compounds of Formula (1) and Formula (II) are provided:

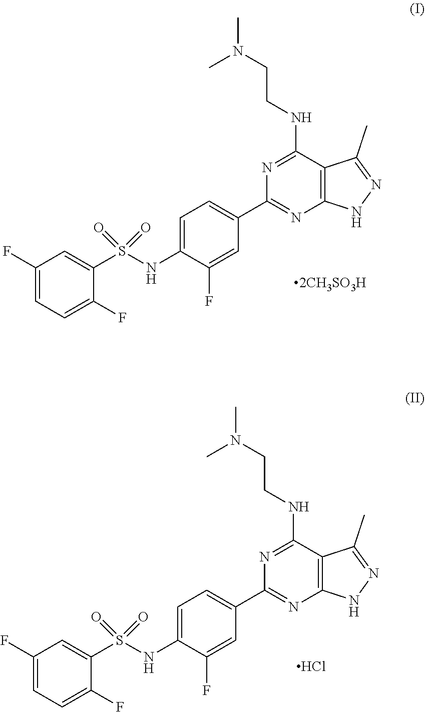 [SMILES: Cc1n[nH]c2nc(-c3ccc(NS(=O)(=O)c4cc(F)ccc4F)c(F)c3)nc(NCCN(C)C)c12 ; Cc1n[nH]c2nc(-c3ccc(NS(=O)(=O)c4cc(F)ccc4F)c(F)c3)nc(NCCN(C)C)c12 ; Cl]

Crystalline forms of the compounds of Formula (1) and Formula (II) are also provided. The compounds of Formula (1) and Formula (II), and crystalline forms thereof can be used for the treatment of several conditions linked to the inhibition of SGK-1, such as a cardiovascular disease selected from the group consisting of Long QT syndrome, heart failure, arrhythmia such as atrial fibrillation, ischemic injury, ischemic infarction, cardiac fibrosis, vascular proliferation, restenosis, dilated cardiomyopathy, and stent failure; cancer; epilepsy; Parkinson's disease; and Lafora disease.

### CROSS-REFERENCE TO RELATED APPLICATION

The present application is a continuation application of International Patent Application No. PCT/CA2023/050930 filed Jul. 11, 2023, which application claims priority from U.S. Provisional Application No. 63/368,208 filed Jul. 12, 2022, the content of which is hereby incorporated by reference in its entirety.

### TECHNICAL FIELD

The technical field relates to salts of the compound N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide and their crystalline forms, as well as pharmaceutical compositions, therapeutic uses thereof and processes of manufacture.

### BACKGROUND

Long QT syndrome (LQTS) is a condition of the heart's electrical system, in which repolarization of the heart after a heartbeat is affected. LQTS results in an increased risk of an irregular heartbeat which can result in fainting, drowning or even sudden death. Several genetic causes for LQTS have been identified, and a majority of mutations are seen in genes encoding for three main cardiac ion channels (KCNQ1, KCNH2 and SCN5a).

There are several existing treatment options for LQTS, such as the use of beta-blockers that slow the heart rate by reducing the effect of adrenaline on the heart, surgery on the nerves that regulate the heartbeat, and/or the use of an implantable cardioverter defibrillator. However, none of the existing treatment options address the underlying mechanistic problem.

Serine/threonine-protein kinase (SGK-1) (also known as serum/glucocorticoid-regulated kinase 1) is a protein kinase that plays a role in a cell's response to stress. SGK-1 activates certain potassium, sodium, and chloride channels. For instance, SGK-1 is known to regulate the myo-inositol transporter during osmotic stress. Several challenges remain in the development of an SGK-1 inhibitor for the treatment of heart conditions such as LQTS.

### SUMMARY

Compounds of Formula (I) and Formula (II) are provided:

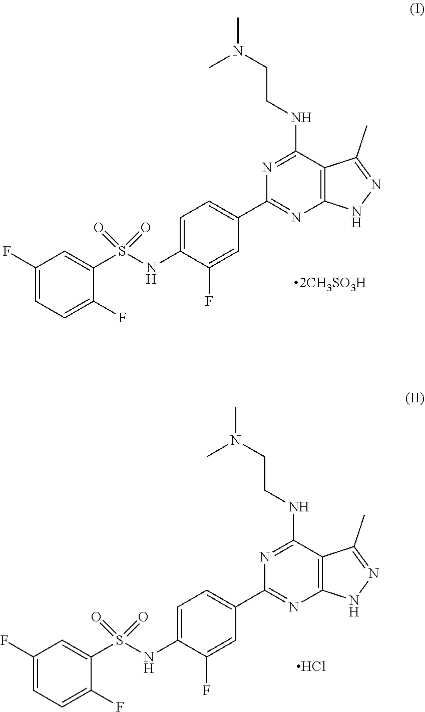 [SMILES: Cc1n[nH]c2nc(-c3ccc(NS(=O)(=O)c4cc(F)ccc4F)c(F)c3)nc(NCCN(C)C)c12 ; Cc1n[nH]c2nc(-c3ccc(NS(=O)(=O)c4cc(F)ccc4F)c(F)c3)nc(NCCN(C)C)c12 ; Cl]

Crystalline forms of the compounds of Formula (I) and Formula (II) are also provided. The compounds of Formula (I) and Formula (II), and crystalline forms thereof can be used for the treatment of several conditions linked to the inhibition of SGK-1, such as a cardiovascular disease selected from the group consisting of Long QT syndrome, heart failure, arrhythmia such as atrial fibrillation, ischemic injury, ischemic infarction, cardiac fibrosis, vascular proliferation, restenosis, dilated cardiomyopathy, and stent failure; cancer; epilepsy; Parkinson's disease; and Lafora disease.

### BRIEF DESCRIPTION OF THE FIGURES

FIG. 1 is an XRPD overlay of Compound 1 (mixture of Material A and Form B) and the Form B of Compound 1;

FIG. 2 is an Expanded XRPD of Compound 1 with allowed peak positions from the indexing solution of Form B (Compound 1);

FIG. 3 is a ¹H NMR spectrum of Compound 1 in DMSO-d6;

FIG. 4 is a ¹H NMR spectrum of Compound 2 in DMSO-d6;

FIG. 5a is an XRPD overlay of Compound 2 (mono-formate salt, anhydrous—Form A) and Compound 1;

FIG. 5b is an XRPD pattern of Compound 2 (mono-formate salt, anhydrous—Form A);

FIG. 6 is an indexing solution of Compound 2 (mono-formate salt, anhydrous—Form A);

FIG. 7 are TGA and DSC thermograms of Compound 2 (mono-formate salt, anhydrous Form A);

FIG. 8 is an XRPD pattern of Compound 3, mono-hydrochloride salt, unsolvated—Form A;

FIG. 9 is an indexing solution of Compound 3, mono-hydrochloride salt, unsolvated—Form A;

FIG. 10 is a ¹H NMR spectra overlay of Compound 1 and Compound 3 (mono-hydrochloride salt unsolvated, Form A) in DMSO-d6;

FIG. 11 are TGA and DSC thermograms for Compound 3 (mono-hydrochloride salt unsolvated, Form A);

FIG. 12 is an XRPD overlay of Compound 4 (Di-mesylate salt, unsolvated—Form A), Compound 1 and Compound 1 Form C;

FIG. 13 is an XRPD pattern of Compound 4 (Di-mesylate salt, unsolvated—Form A);

FIG. 14 is an indexing solution of Compound 4 (Di-mesylate salt, unsolvated—Form A);

FIG. 15 is a ¹H NMR spectrum of Compound 4 in DMSO-d6 (Di-mesylate salt, unsolvated—Form A) FIG. 16 are TGA and DSC thermograms for Compound 4 (Di-mesylate salt, unsolvated Form A);

FIG. 17 is a DVS isotherm and results table for Compound 2 (Formate salt, anhydrous Form A);

FIG. 18 is an XRPD overlay of Compound 2 (Formate salt, anhydrous—Form A) pre- and post-DVS;

FIG. 19 is a DVS isotherm and results table for Compound 3 (Hydrochloride salt, unsolvated—Form A);

FIG. 20 is an XRPD overlay of Compound 3 (Hydrochloride salt, unsolvated—Form A) pre- and post-DVS;

FIG. 21 is a DVS isotherm and results table for Compound 4 (Di-mesylate salt, unsolvated—Form A);

FIG. 22 is an XRPD overlay of Compound 4 (Di-mesylate salt, unsolvated—Form A) pre- and post-DVS;

FIG. 23 is an XRPD pattern of Compound 1 (Free Form—Form D);

FIG. 24 are TGA and DSC thermograms for Compound 1 (Free Form—Form D);

FIG. 25 is an XRPD pattern of Compound 3 (Form B);

FIG. 26 are TGA and DSC thermograms for Compound 3 (Form B);

FIG. 27 is an XRPD pattern of Compound 3 (Form C);

FIG. 28 are TGA and DSC thermograms for Compound 3 (Form C);

FIG. 29 is an XRPD pattern of Compound 3 (Form D);

FIG. 30 are TGA and DSC thermograms for Compound 3 (Form D);

FIG. 31 is an XRPD pattern of Compound 3 (Form E);

FIG. 32 are TGA and DSC thermograms for Compound 3 (Form E);

FIG. 33 is an overlay of Compound 1 (from top to 2^{nd} line: Free Form E/Free Form D) and Compound 3 (following from 3^{rd} line to bottom Form E/Form D/Form C);

FIG. 34 is a DVS plot of Compound 3 (Form D);

FIG. 35 is a DVS mass plot of Compound 3 (Form D);

FIG. 36 is an XRPD overlay of Compound 3 (Form D) pre- (down) and post-DVS (up);

FIG. 37 is an XRPD pattern of Compound 4 (Form B);

FIG. 38 are TGA and DSC thermograms for Compound 4 (Form B);

FIG. 39 is an XRPD pattern of Compound 4 (Form C);

FIG. 40 are TGA and DSC thermograms for Compound 4 (Form C);

FIG. 41 is an XRPD pattern of Compound 4 (Form D);

FIG. 42 are TGA and DSC thermograms for Compound 4 (Form D);

FIG. 43 is an XRPD pattern of Compound 4 (Form E);

FIG. 44 is an XRPD pattern of Compound 4 (Form F);

FIG. 45 are TGA and DSC thermograms for Compound 4 (Form F);

FIG. 46 is an XRPD pattern of Compound 4 (Form G);

FIG. 47 are TGA and DSC thermograms for Compound 4 (Form G);

FIG. 48 is a DVS plot of Compound 4 (Form B);

FIG. 49 is an XRPD overlay of Compound 4 (Form B) pre- (down) and post-DVS (up);

FIG. 50 is a DVS plot of Compound 4 (Form D); and

FIG. 51 is an XRPD overlay of Compound 4 (Form D) pre- (down) and post-DVS (up).

### DETAILED DESCRIPTION

### Definitions

The term “stable”, as used herein, includes chemical stability and/or solid-state stability. A compound is considered chemically stable when the compound can be stored in an isolated solid form, or in the form of a solid formulation in which it may be provided in admixture with pharmaceutically acceptable carriers, diluents or adjuvants, under normal storage conditions, without any significant degree of chemical degradation or decomposition.

A compound is considered to have solid-state stability when the compound can be stored in an isolated solid form, or in the form of a solid formulation in which it may be provided in admixture with pharmaceutically acceptable carriers, diluents or adjuvants, under normal storage conditions, without any significant degree of solid state transformation (e.g. crystallisation, recrystallisation, loss of crystallinity, solid state phase transition, hydration, dehydration, deliquescence, solvation or desolvation).

Crystalline forms of solid chemical compounds influence not only their dissolution behavior (i.e. bioavailability) but also their solid-state stability. One way of comparing the solid-state stability of crystalline forms is to evaluate the relative “thermodynamic stability” of the crystalline forms. To evaluate the thermodynamic stability of crystalline forms, typical techniques include, but are not limited to, slurrying, slow evaporation, slow cooling, slow antisolvent addition, or a combination of these methods. Calorimetry techniques (e.g., Differential Scanning Calorimetry) can also be used to measure thermal events and phase transitions across a wide temperature range, and a comparison between the crystalline forms can give an indication as to their relative thermodynamic stability.

The expression “pharmaceutically acceptable carrier or excipient”, as used herein, includes without limitation any adjuvant, carrier, glidant, sweetening agent, diluent, preservative, dye/colorant, flavor enhancer, surfactant, wetting agent, dispersing agent, suspending agent, stabilizer, isotonic agent, solvent or emulsifier which is known as being acceptable for pharmaceutical use in humans or domestic animals.

The expression “pharmaceutical composition”, as used herein, refers to the formulation of a compound and a pharmaceutically acceptable carrier or excipient.

The term “about”, as used herein, generally means within an acceptable standard error of the mean, when considered by a person skilled in the art. For example, depending on the value or range considered, the term “about” can mean within 10%, within 5%, or within 1% of the value or range.

As used herein, the term “hydrate” refers to a crystalline form of a molecule that further comprises molecules of water incorporated into the crystalline lattice structure. The water molecules in the hydrate may be present in a regular arrangement and/or a non-ordered arrangement. The hydrate may comprise either a stoichiometric or nonstoichiometric amount of the water molecules. For example, a hydrate with a nonstoichiometric amount of water molecules may result from partial loss of water from the hydrate.

As used herein, the term “non-stoichiometric hydrate” refers to a hydrate that exists as channel structure with the water packed throughout the crystal lattice thus forming in both stoichiometric and nonstoichiometric phases.

As used herein, the terms “anhydrate” or “anhydrous” refer to a crystalline form of a molecule per se that does not further comprise molecules of water incorporated into the crystalline lattice structure.

As used herein, the term “solvate” refers to a crystalline form of a molecule that further comprises molecules of a solvent or solvents incorporated into the crystalline lattice structure. The solvent molecules in the solvate may be present in a regular arrangement and/or a non-ordered arrangement. The solvate may comprise either a stoichiometric or nonstoichiometric amount of the solvent molecules. For example, a solvate with a nonstoichiometric amount of solvent molecules may result from partial loss of solvent from the solvate. The solvent can include various organic solvents. It should also be understood that a “solvate” can include a single solvent, a mixture of solvents or a mixture of a solvent (or solvents) and water.

The term “substantially the same”, used herein to describe X-ray diffraction patterns, is meant to include patterns in which peaks are within a standard deviation of ±0.2° 2θ or an X-ray diffraction pattern comprising least 4, 5, 6, 7, 8, 9, 10, 11, 12, 13, 14, 15 or 16 peaks in common with the referenced pattern. Further, a person skilled in the art will appreciate that relative peak intensities will show inter-apparatus variability as well as variability due to degree of crystallinity, preferred orientation, prepared sample surface, and other factors. As such, the relative peak intensities should be taken as a qualitative measure.

The present description provides salt screening experiments from N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide (Compound 1) and the crystalline forms thereof. In particular, the present description provides the following compound of Formula I and Formula II:

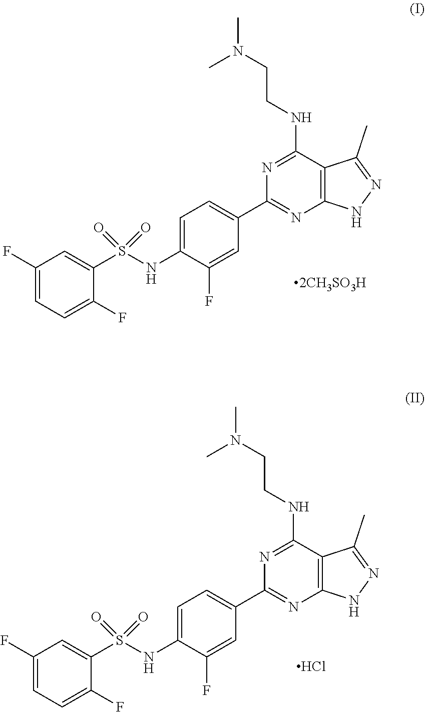 [SMILES: Cc1n[nH]c2nc(-c3ccc(NS(=O)(=O)c4cc(F)ccc4F)c(F)c3)nc(NCCN(C)C)c12 ; Cc1n[nH]c2nc(-c3ccc(NS(=O)(=O)c4cc(F)ccc4F)c(F)c3)nc(NCCN(C)C)c12 ; Cl]

The structure depicted for the compound of Formula I or Formula II is also meant to include all tautomeric forms of the compound of Formula I or Formula II. Additionally, structures depicted herein are also meant to include compounds that differ only in the presence of one or more isotopically enriched atoms. For example, compounds having the structure of the compound of Formula I except for the replacement of hydrogen by deuterium or tritium, or the replacement of a carbon by a ¹³C- or ¹⁴C-enriched carbon are within the scope of the present description.

The term “substantially pure”, when used in reference to a crystalline form of the compound of Formula I or Formula II, is meant to include a crystalline form which has a purity that is greater than about 90%. This means that the crystalline form may not contain more than about 10% of any other compound, and in particular, does not contain more than about 10% of any other crystalline form of the compound of Formula I or Formula II. Preferably, the term “substantially pure” means a crystalline form which has a purity that is greater than about 95%. This means that the crystalline form may not contain more than about 5% of any other compound, and in particular, does not contain more than about 5% of any other crystalline form of the compound of Formula I or Formula II. More preferably, the term “substantially pure” means a crystalline form which has a purity that is greater than about 99%. This means that the crystalline form may not contain more than about 1% of any other compound, and in particular, does not contain more than about 1% of any other crystalline form of the compound of Formula I or Formula II.

The term “solid mixture” when used in reference to the compounds of the present description, refers to a mixture of crystalline forms. For example, a solid mixture can include at least two different crystalline forms.

XRPD data were obtained using a PANalytical X'Pert PRO MPD or a PANanalytical Empyrean X-ray powder diffractometers, using an incident beam of Cu radiation produced by an Optix long, fine-focus source. The radiation used was Cu Kα (λ=1.5405929 Å). It should be understood that the 2θ values listed herein are dependent on the Form of radiation used, and that a person skilled in the art would understand that the XRPD of a given crystalline form will exhibit different 2θ values if a different radiation is used (e.g., a molybdenum radiation).

As used herein the terms “crystalline Form” or “polymorph” refers to crystal structure of a compound, having the same chemical composition but different spatial arrangements of the molecules, atoms, and/or ions forming the crystal structure.

The compounds of the present description may exist in solvated, for example hydrated, as well as unsolvated forms. Typically, but not absolutely, the salts of the compounds of the present description are pharmaceutically acceptable salts. Salts encompassed within the term “pharmaceutically acceptable salts” refer to non-toxic salts of the compounds of the present description.

Examples of suitable pharmaceutically acceptable salts include inorganic acid addition salts such as chloride, bromide, sulfate, phosphate, and nitrate; organic acid addition salts such as acetate, galactarate, propionate, succinate, lactate, glycolate, malate, tartrate, citrate, maleate, fumarate, methanesulfonate, p-toluenesulfonate, and ascorbate; salts with acidic amino acid such as aspartate and glutamate; alkali metal salts such as sodium salt and potassium salt; alkaline earth metal salts such as magnesium salt and calcium salt; ammonium salt; organic basic salts such as trimethylamine salt, triethylamine salt, pyridine salt, picoline salt, dicyclohexylamine salt, and N,N′-dibenzylethylenediamine salt; and salts with basic amino acid such as lysine salt and arginine salt. The salts may be in some cases hydrates or ethanol solvates.

### Salts Formation Experiments of Compound 1 and Crystalline Forms

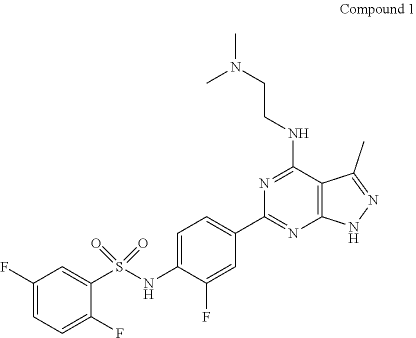 [SMILES: Cc1n[nH]c2nc(-c3ccc(NS(=O)(=O)c4cc(F)ccc4F)c(F)c3)nc(NCCN(C)C)c12]

Salt formation experiments were conducted using a variety of acids (i.e. formic acid, hydrochloric acid, phosphoric acid, L-tartaric acid, sulfuric acid, succinic acid, maleic acid, citric acid, L-Lysine and methanesulfonic acid) with Compound 1 (N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide).

### Formate Salt

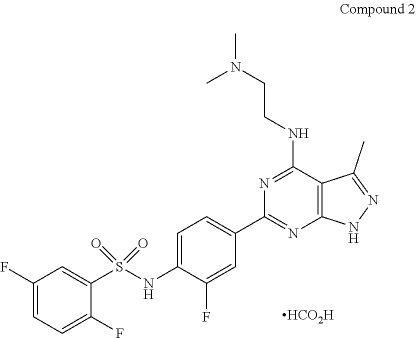 [SMILES: Cc1n[nH]c2nc(-c3ccc(NS(=O)(=O)c4cc(F)ccc4F)c(F)c3)nc(NCCN(C)C)c12 ; "·HCO2H"]

The formate salt (Compound 2) can be prepared by combining Compound 1 with 2 molar equivalents of formic acid in MeOH at 55° C. Upon dissolution of the solids the solution was cooled to room temperature and stirred for 3 days. Compound 2 was isolated from the previous slurry as a unique crystalline material.

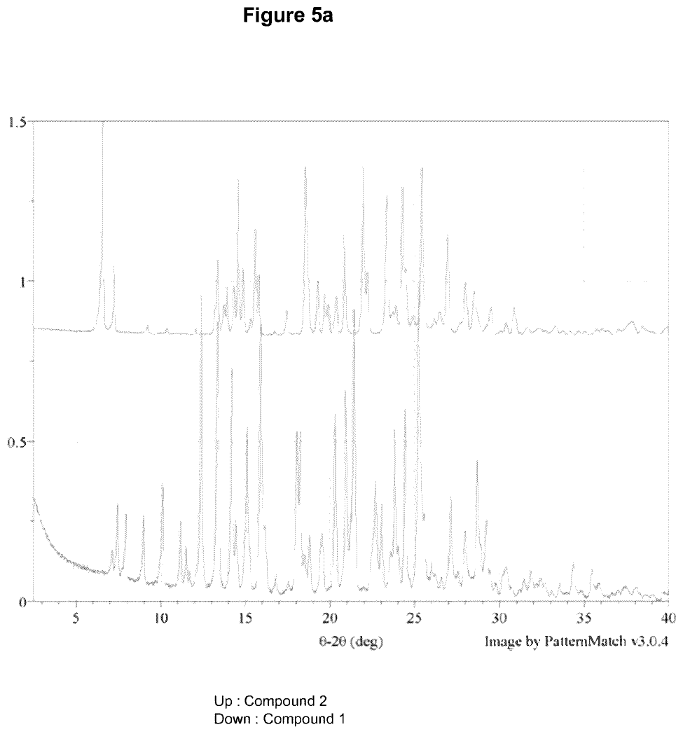

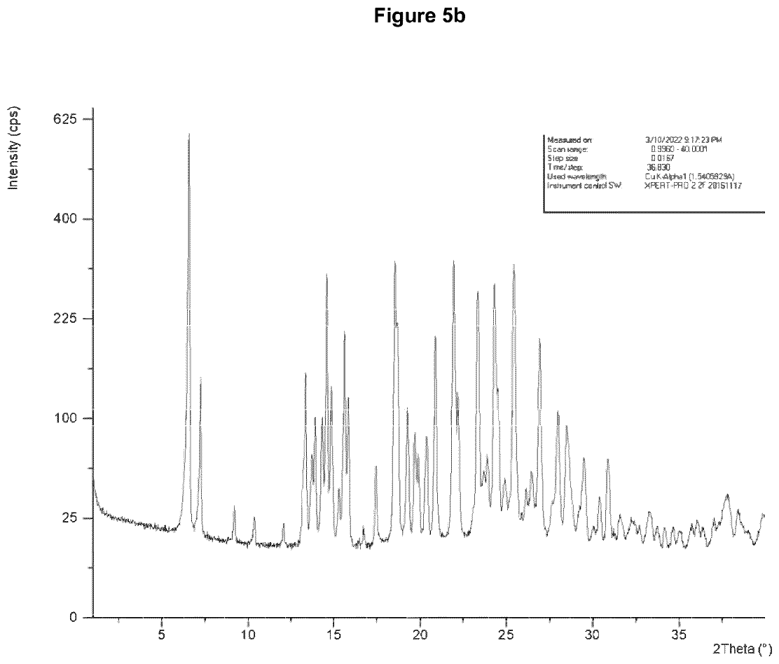

Compound 2 exhibits an XRPD pattern (FIGS. 5a and 5b) having characteristic peaks expressed in degrees 2θ (±0.2° 2θ) at 6.58 and 21.97. The XRPD pattern of Compound 2 can also exhibit further characteristic peaks expressed in degrees 2θ (±0.2° 2θ) at 18.55 and 25.44. The XRPD pattern of Compound 2 can also exhibit further characteristic peaks expressed in degrees 2θ (±0.2° 2θ) at 14.59 and 24.31. The XRPD pattern of Compound 2 can also exhibit further characteristic peaks expressed in degrees 2θ (±0.2° 2θ) at 23.35 and 18.68. The XRPD pattern of Compound 2 can also exhibit further characteristic peaks expressed in degrees 2θ (±0.2° 2θ) at 15.61 and 20.88.

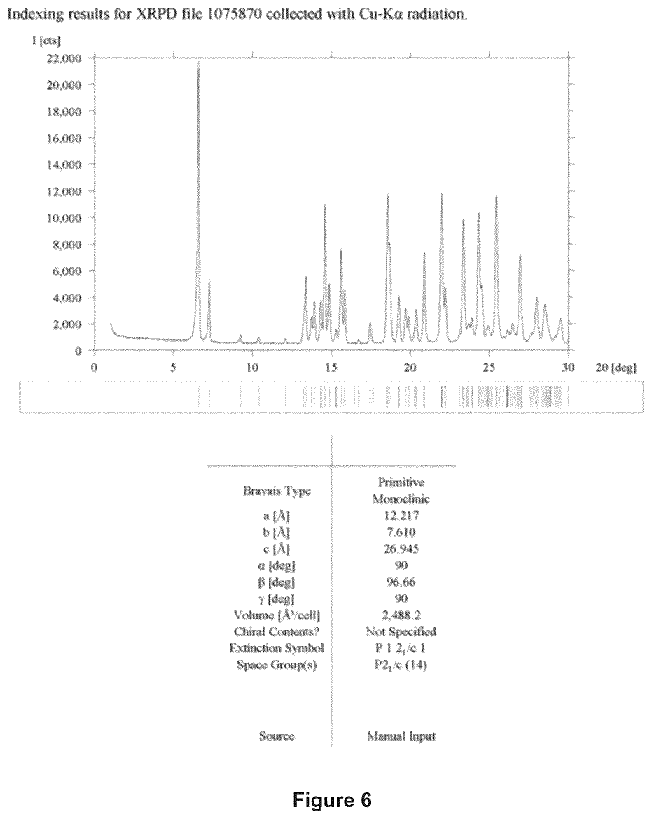

Successful indexing of a pattern (FIG. 6) indicates that the sample is composed primarily or exclusively of a single crystalline phase. The volume from the indexing solution was consistent with an anhydrous, mono-formate salt attributed to crystalline Form A of Compound 2.

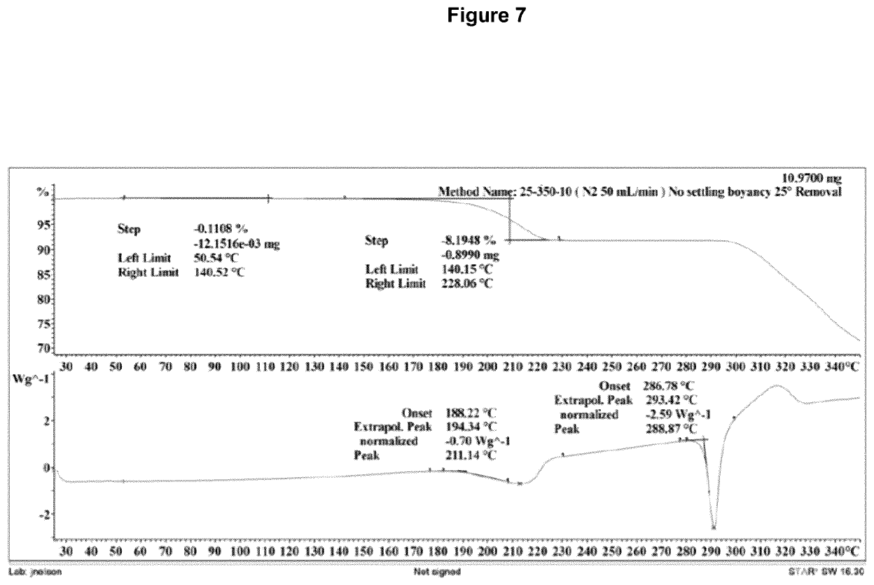

According to DSC, Form A shows a broad endotherm with an onset of 188° C. and a peak temperature of 211° C. Form A also displays an endotherm that has a peak temperature at about 289° C. with an onset of 287° C. The TGA analysis of Form A shows a weight loss of about 0.1% from 51° C. to 141° C. and weight loss of about 8.2% from 140° C. to 228° C. (FIG. 7)

### Hydrochloride Salt

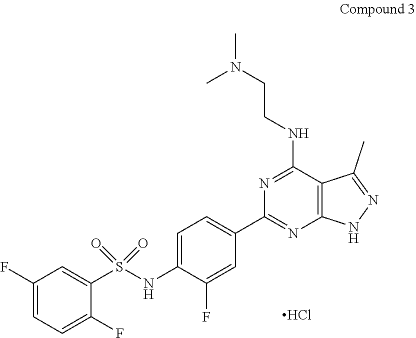 [SMILES: Cc1n[nH]c2nc(-c3ccc(NS(=O)(=O)c4cc(F)ccc4F)c(F)c3)nc(NCCN(C)C)c12 ; Cl]

### Also Referred to Herein as Compound of Formula II

### Form A

Crystalline Form A is a mono-HCl unsolvated.

The hydrochloride salt (Compound 3) can be prepared by combining Compound 1 with 2 molar equivalents of hydrochloric acid in MeOH at 55° C. Stirring for several days afforded a mixture of Compound 3 as a unique crystalline material (Form A) and minor additional unknown XRPD peaks. The solid mixture was slurried in acetone for 2 days at ambient temperature and afforded a mixture of Compound 3 as a unique crystalline material (Form A) and minor additional unidentified XRPD peaks.

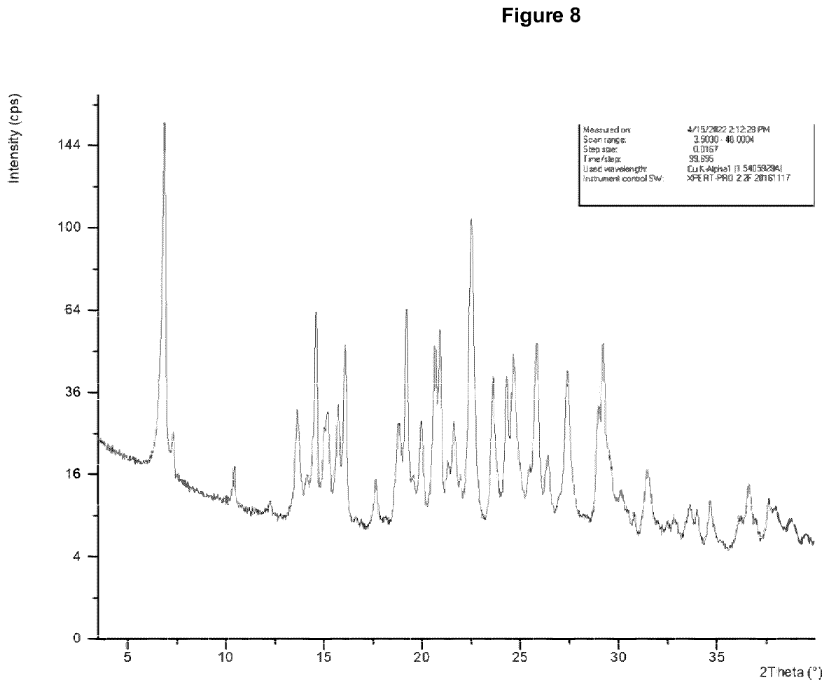

The hydrochloride salt (Compound 3) can be also prepared by combining Compound 1 with 2 molar equivalents of hydrochloric acid in MeOH at room temperature. The slurry was then stirred at 60° C. and water added. Additional stirring for 12 days at room temperature afforded a mixture of Compound 3 as a unique crystalline material (Form A) and minor additional unknown XRPD peaks. The solid mixture was then slurried in water (RT, 1 day stirring) and afforded Form A as a single crystalline phase. Form A of Compound 3 was identified as the unsolvated mono-hydrochloride salt, as shown by the XRPD pattern on FIG. 8.

Form A exhibits an XRPD pattern (FIG. 8) having characteristic peaks expressed in degrees 2θ (±0.2° 2θ) at 6.81 and 14.53. The XRPD pattern of Form A can also exhibit further characteristic peaks expressed in degrees 2θ (±0.2° 2θ) at 25.76 and 24.59. The XRPD pattern of Form A can also exhibit further characteristic peaks expressed in degrees 2θ (±0.2° 2θ) at 22.45 and 19.13. The XRPD pattern of Form A can also exhibit further characteristic peaks expressed in degrees 2θ (±0.2° 2θ) at 23.56 and 27.34. The XRPD pattern of Form A can also exhibit further characteristic peaks expressed in degrees 2θ (±0.2° 2θ) at 15.12 and 20.83.

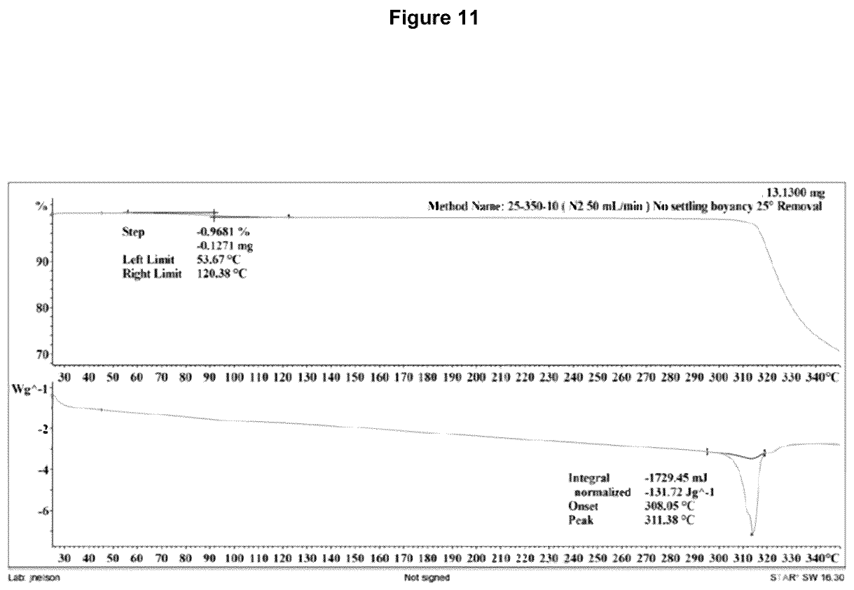

According to DSC, Form A shows an endotherm with an onset of 308° C. and a peak temperature of 311° C. The TGA analysis of Form A shows a weight loss of about 1.0% from 54° C. to 120° C. (FIG. 11).

### Form B

Crystalline Form B is an anhydrate.

Form B can be obtained from suspending Compound 1 into water (e.g., 6 vol.) to obtain a suspension and adjusting the pH of the suspension between 3 and 4 with HCl (e.g. HCl 3N), at 20-30° C.; Stirring the suspension for 2-4 hours, or for 3-4 hours, or for about 3 hours at 20-30° C.; filtering the suspension to obtain a filter cake and washing the filter cake with water (e.g., 1 vol.); suspending the washed filter cake into a 5% solution NaHCO₃ (e.g., 6 vol.) and stirring the suspension for 4-6 hours, or for 4-5 hours, or for about 4.5 hours at 20-30° C.; filtering to obtain a filter cake and washing the filter cake with water (e.g., 1 vol.); suspending the washed filter cake in MeOH/water (1/4, 5 vol.), for 7-8 hours, or for about 7.5 hours at 20-30° C.; filtering the suspension to obtain a filter cake and washing the filter cake with water (1 vol.); and drying the filter cake at 55-65° C. to obtain Compound 3, Form B.

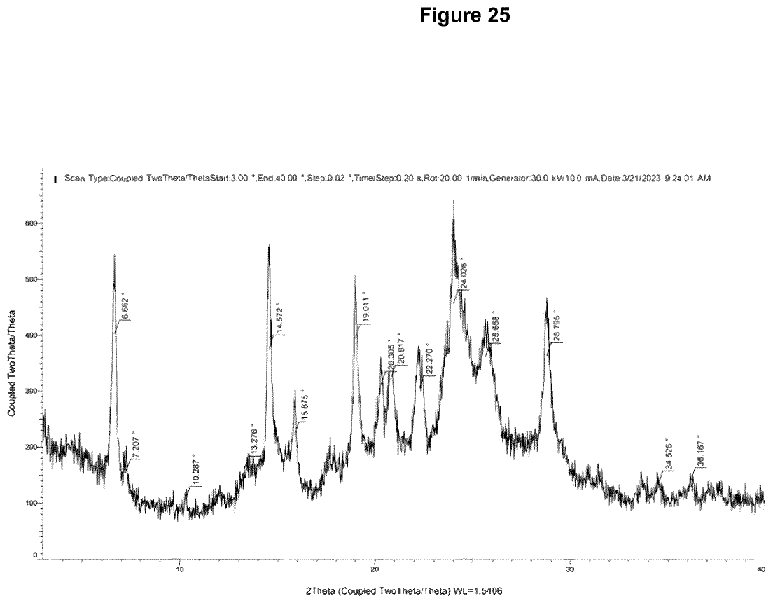

Form B exhibits an XRPD pattern (FIG. 25) having characteristic peaks expressed in degrees 2θ (±0.2° 2θ) at 6.7 and 14.6. The XRPD pattern of Form B can also exhibit further characteristic peaks expressed in degrees 2θ (±0.2° 2θ) at 24.0 and 19.0. The XRPD pattern of Form B can also exhibit further characteristic peaks expressed in degrees 2θ (±0.2° 2θ) at 28.8 and 25.7. The XRPD pattern of Form B can also exhibit further characteristic peaks expressed in degrees 2θ (±0.2° 2θ) at 20.8 and 20.3. The XRPD pattern of Form B can also exhibit further characteristic peaks expressed in degrees 2θ (±0.2° 2θ) at 15.9 and 22.3.

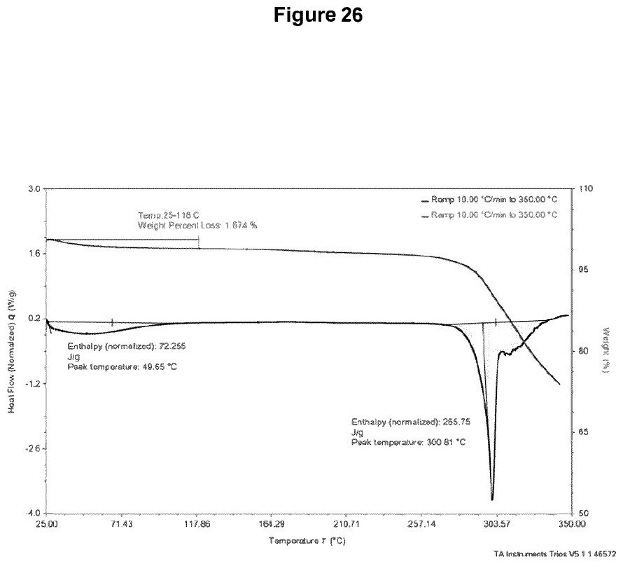

According to DSC, Form B shows two endothermic peaks observed at 49.65° C. and 300.81° C., corresponding to release of moisture and melting/decomposition respectively. The TGA analysis of Form B shows a weight loss of about 1.6% from 25° C. to 118° C. (FIG. 26).

### Form C

Crystalline Form C is an anhydrate.

Form C can be obtained from MeOH by slurrying Compound 3, Form B at 50° C. for 4 days. Form C was recovered by filtration and dried under vacuum at 50° C. for 3 hours. The recovery yield is 74.9%.

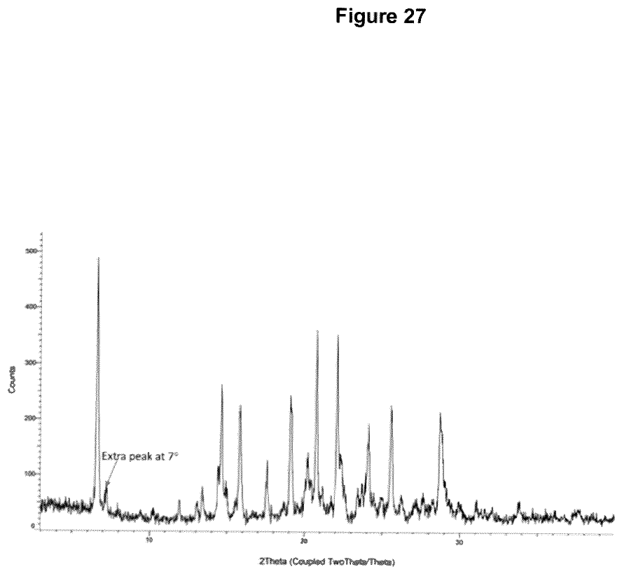

Form C exhibits an XRPD pattern (FIG. 27) having characteristic peaks expressed in degrees 2θ (±0.2° 2θ) at 6.6 and 22.1. The XRPD pattern of Form C can also exhibit further characteristic peaks expressed in degrees 2θ (±0.2° 2θ) at 20.8 and 14.7. The XRPD pattern of Form C can also exhibit further characteristic peaks expressed in degrees 2θ (±0.2° 2θ) at 15.8 and 25.6. The XRPD pattern of Form C can also exhibit further characteristic peaks expressed in degrees 2θ (±0.2° 2θ) at 24.1 and 19.1. The XRPD pattern of Form C can also exhibit further characteristic peaks expressed in degrees 2θ (±0.2° 2θ) at 28.7 and 22.3.

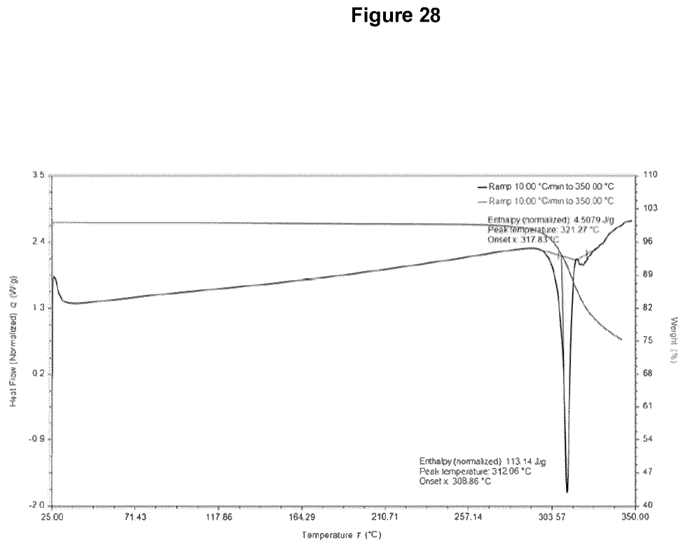

According to DSC, Form C shows an endotherm with an onset of 308.86° C. and a peak temperature of 312.06° C. The TGA analysis of Form C shows no weight loss prior to melting (FIG. 28).

### Form D

Crystalline Form D is an anhydrate.

Form D (Compound of Formula II) can be obtained from DMSO/Water by reverse anti-solvent precipitation. 150 mg of Compound 3 were dissolved in 2 mL of DMSO at 70° C. and then the solution was filtered at room temperature. The filtrate was charged in 20 mL of water (anti-solvent), and the suspension was stirred at room temperature for 4 days. Compound 3, Form D was collected by filtration and dried under vacuum at 50° C. for 3 hours. The recovery yield is 73.5%.

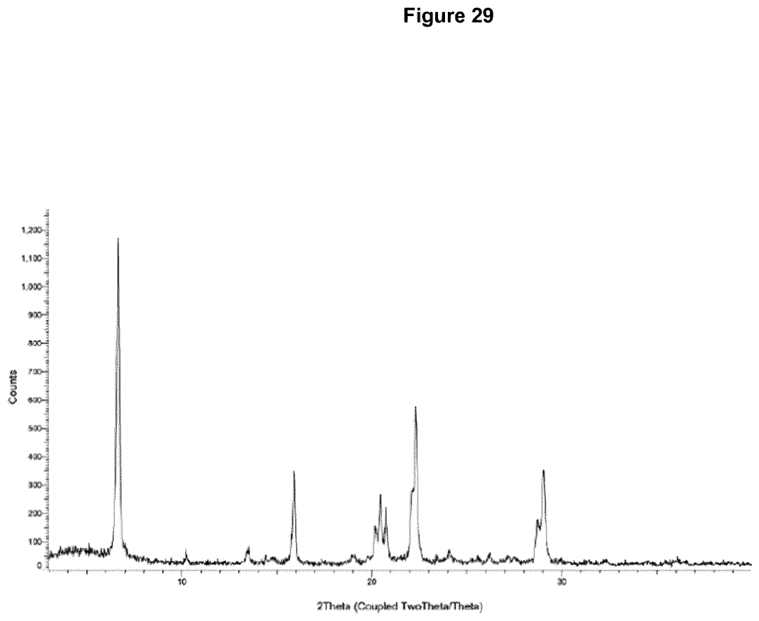

Form D exhibits an XRPD pattern (FIG. 29) having characteristic peaks expressed in degrees 2θ (±0.2° 2θ) at 6.7 and 22.3. The XRPD pattern of Form D can also exhibit further characteristic peaks expressed in degrees 2θ (±0.2° 2θ) at 29.0 and 15.9. The XRPD pattern of Form D can also exhibit further characteristic peaks expressed in degrees 2θ (±0.2° 2θ) at 20.5 and 20.7. The XRPD pattern of Form D can also exhibit further characteristic peaks expressed in degrees 2θ (±0.2° 2θ) at 28.7 and 20.2. The XRPD pattern of Form D can also exhibit further characteristic peaks expressed in degrees 2θ (±0.2° 2θ) at 13.5 and 26.2. The XRPD pattern of Form D can also exhibit further characteristic peaks expressed in degrees 2θ (±0.2° 2θ) at 23.5 and 36.1. The XRPD pattern of Form D can also exhibit further characteristic peaks expressed in degrees 2θ (±0.2° 2θ) at 24.1 and 10.2. The XRPD pattern of Form D can also exhibit further characteristic peaks expressed in degrees 2θ (±0.2° 2θ) at 25.6 and 19.0. The XRPD pattern of Form D can also exhibit further characteristic peaks expressed in degrees 2θ (±0.2° 2θ) at 27.2 and 32.3.

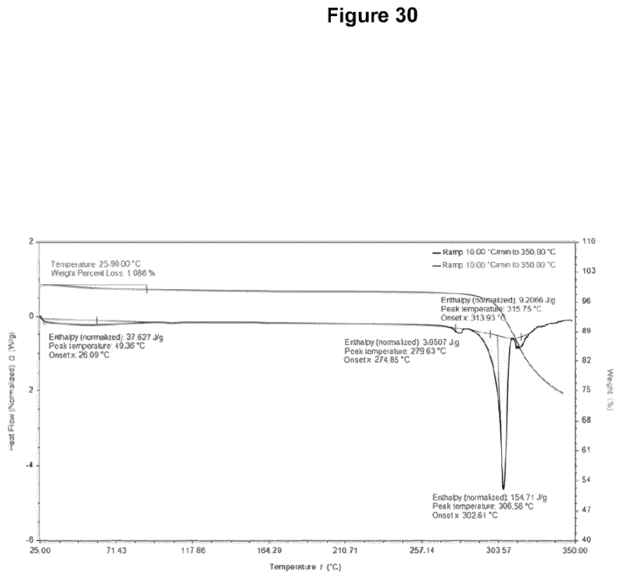

According to DSC, Form D shows four endothermic peaks observed at 49.36° C. (onset 26.09° C.), 279.63° C. (onset 274.85° C.), 306.56° C. (onset 302.61° C.) and 315.75° C. (onset 313.93° C.). The first peak (at 49.36° C.) is attributed to DMSO and moisture. The sharp peak at 306.56° C. is attributed to melting and the peak at 315.75° C. to decomposition. The TGA analysis of Form D shows a weight loss of about 1.1% from 25° C. to 90° C. (FIG. 30)

### Form E

Crystalline Form E is a mono-DMA solvate.

Form E can be obtained from NMP/MTBE, DMA/MTBE, DMA/EA, DMA/IPAC or DMA/MIBK by anti-solvent precipitation, and from DMA/MTBE, DMA/EA or DMA/MIBK by reverse anti-solvent precipitation.

150 mg of Compound 3, Form B were dissolved in 7 mL of DMA at 70° C. and then the solution was filtered at room temperature. 49 mL of MTBE (anti-solvent) was added in the filtrate in 10 hours. The suspension was stirred at room temperature for 4 days. Compound 3, Form E was collected by filtration and dried under vacuum at 50° C. for 3 hours. The recovery yield is 96.4%.

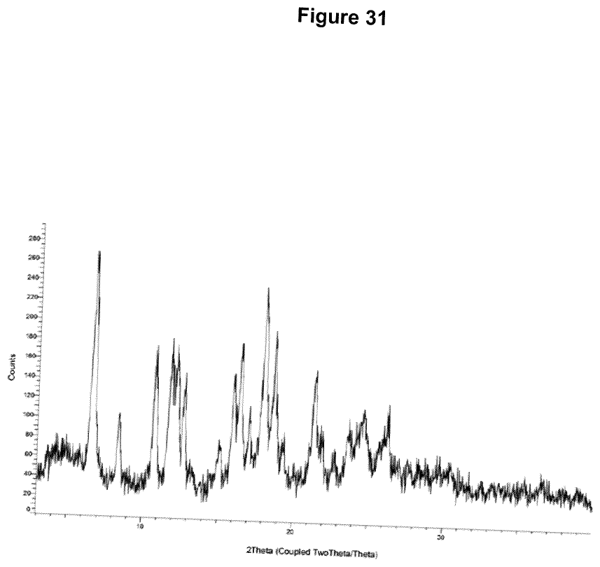

Form E exhibits an XRPD pattern (FIG. 31) having characteristic peaks expressed in degrees 2θ (±0.2° 2θ) at 6.6 and 18.0. The XRPD pattern of Form E can also exhibit further characteristic peaks expressed in degrees 2θ (±0.2° 2θ) at 11.8 and 12.2. The XRPD pattern of Form E can also exhibit further characteristic peaks expressed in degrees 2θ (±0.2° 2θ) at 16.5 and 18.7. The XRPD pattern of Form E can also exhibit further characteristic peaks expressed in degrees 2θ (±0.2° 2θ) at 12.7 and 21.4. The XRPD pattern of Form E can also exhibit further characteristic peaks expressed in degrees 2θ (±0.2° 2θ) at 10.8 and 16.0.

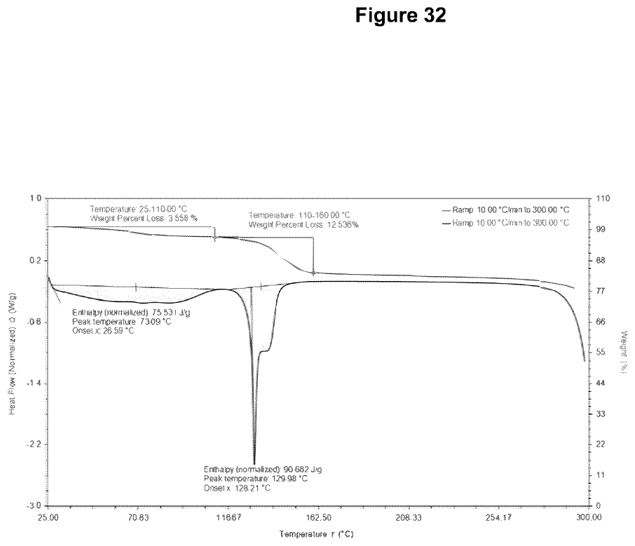

According to DSC, Form E shows two endothermic peaks observed at 73.09° C. (onset 26.59° C.) and at 129.98° C. (onset 128.21° C.). The TGA analysis of Form E shows a weight loss of about 3.6% and 12.5% from 25° C. to 110° C. and 110° C. to 160° C. (FIG. 32).

From the XRPD diffractograms of Compound 3, Forms A, B and C, it appears that Forms A, B and C are made in part of crystalline Form D, and further include other unidentified crystalline material/forms. From the Examples shown below, it appears that Form D is the most stable anhydrate crystalline form of Compound 3 that was identified.

### Mesylate Salt

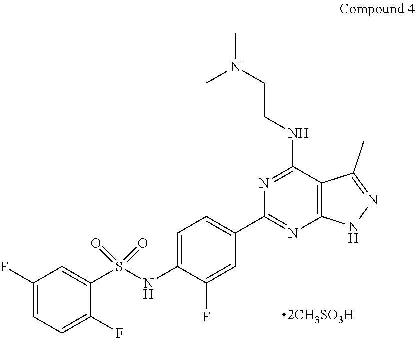 [SMILES: Cc1n[nH]c2nc(-c3ccc(NS(=O)(=O)c4cc(F)ccc4F)c(F)c3)nc(NCCN(C)C)c12 ; "·2CH3SO3H"]

### Also Referred to Herein as Compound of Formula I

The mesylate salt (Compound 4 or Compound of Formula I) can be prepared by combining Compound 1 with 2 molar equivalents of methanesulfonic acid in MeOH at room temperature. Partial slow evaporation followed by stirring at room temperature for several days afforded Compound 4 as a unique crystalline material.

### Form A

Crystalline Form A is an anhydrate.

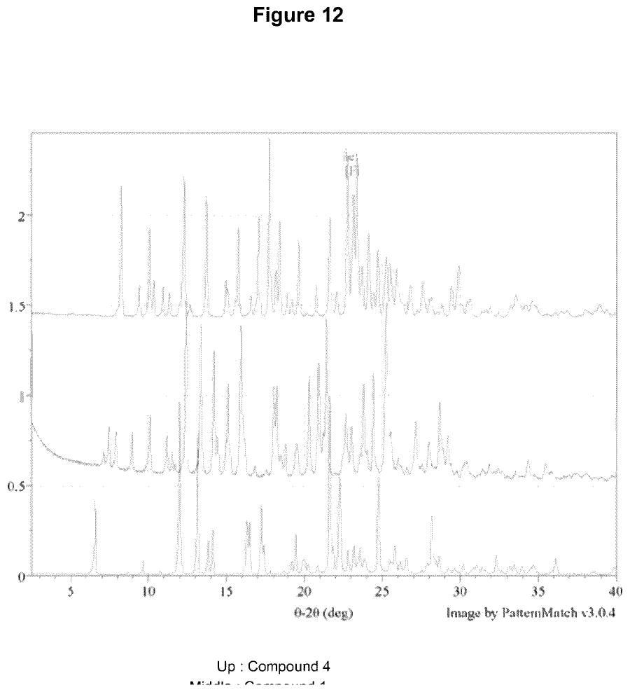

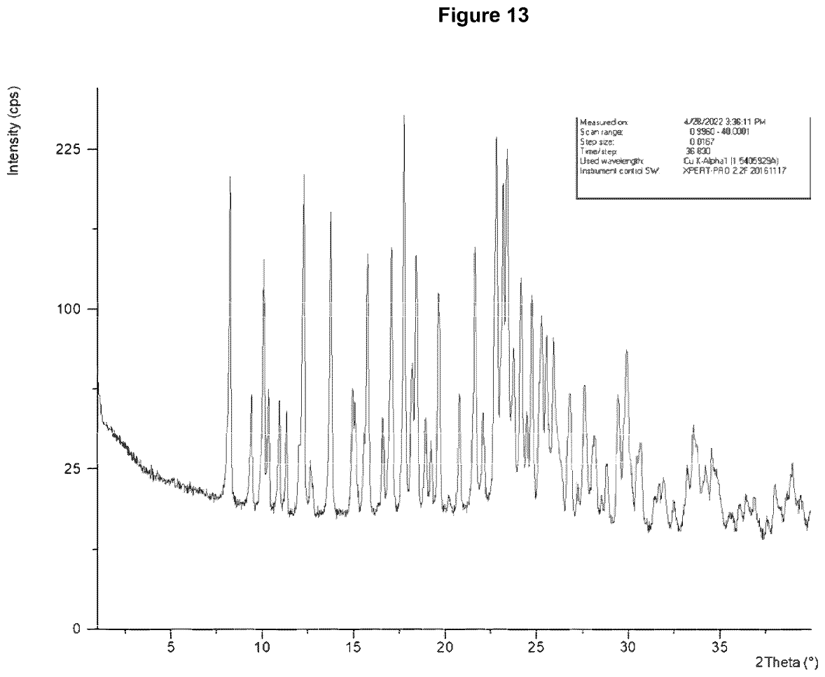

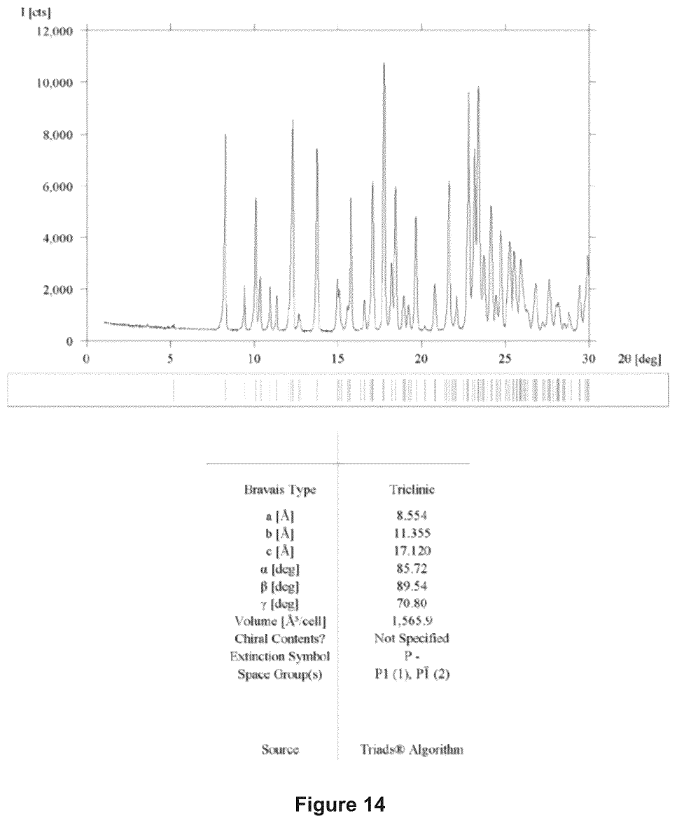

Form A can be prepared by following the procedure reported in Example 6. Form A has an XRPD pattern substantially the same to that shown at FIGS. 12 and 13, indexing of the pattern (FIG. 14) indicated the formation of an unsolvated di-mesylate salt identified as the crystalline Form A of Compound 4.

Form A exhibits an XRPD pattern (FIGS. 12 and 13) having characteristic peaks expressed in degrees 2θ (±0.2° 2θ) at 17.76 and 23.38. The XRPD pattern of Form A can also exhibit further characteristic peaks expressed in degrees 2θ (±0.2° 2θ) at 22.80 and 12.29. The XRPD pattern of Form A can also exhibit further characteristic peaks expressed in degrees 2θ (±0.2° 2θ) at 8.26 and 13.75. The XRPD pattern of Form A can also exhibit further characteristic peaks expressed in degrees 2θ (±0.2° 2θ) at 23.16 and 21.63. The XRPD pattern of Form A can also exhibit further characteristic peaks expressed in degrees 2θ (±0.2° 2θ) at 17.07 and 18.44.

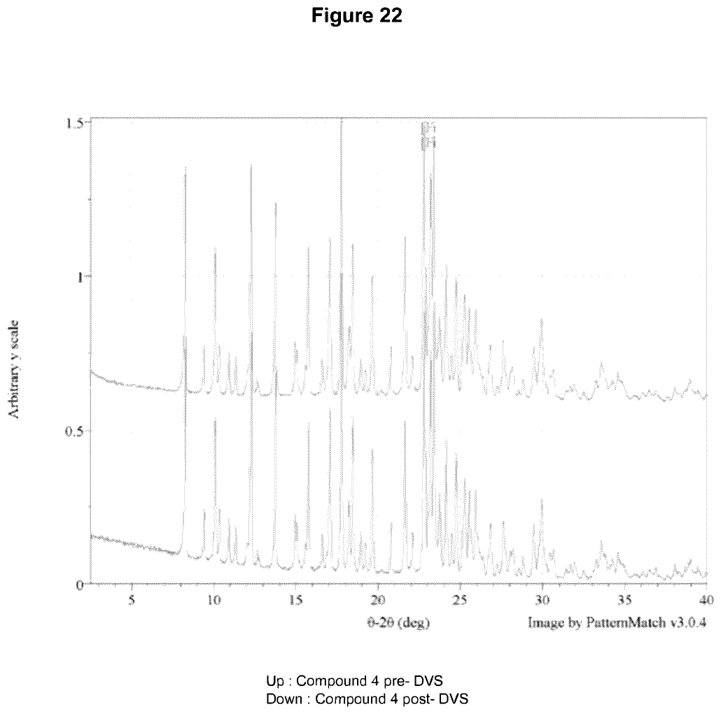

Form A can be subjected to drying in a vacuum oven for a day at 62-72° C. and retain its crystalline form (FIG. 22: pre-DVS XPRD pattern), thus indicating its physical stability under such conditions.

According to DSC, Form A shows a broad endotherm with an onset of 164° C. and a peak temperature of 80° C. Form A also displays two endotherms that have peak temperatures at about 175° C. and 189° C. The TGA analysis of Form A shows a weight loss of about 0.8% from 53° C. to 129° C. (FIG. 16).

### Form B

Crystalline Form B is a dimesylate salt non-stoichiometric hydrate (3.5 eq, at 90% RH).

Form B can be obtained from a variety of conditions such as slurrying, slow cooling and anti-solvent precipitation and are summarized in Example 7.

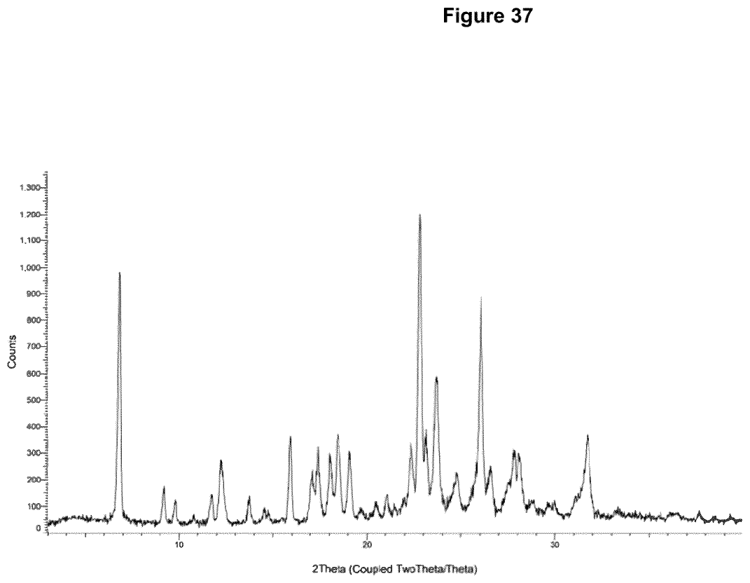

Form B exhibits an XRPD pattern (FIG. 37) having characteristic peaks expressed in degrees 2θ (±0.2° 2θ) at 22.8 and 6.8. The XRPD pattern of Form B can also exhibit further characteristic peaks expressed in degrees 2θ (±0.2° 2θ) at 26.1 and 23.7. The XRPD pattern of Form B can also exhibit further characteristic peaks expressed in degrees 2θ (±0.2° 2θ) at 15.9 and 18.5. The XRPD pattern of Form B can also exhibit further characteristic peaks expressed in degrees 2θ (±0.2° 2θ) at 17.4 and 22.4. The XRPD pattern of Form B can also exhibit further characteristic peaks expressed in degrees 2θ (±0.2° 2θ) at 31.7 and 27.8.

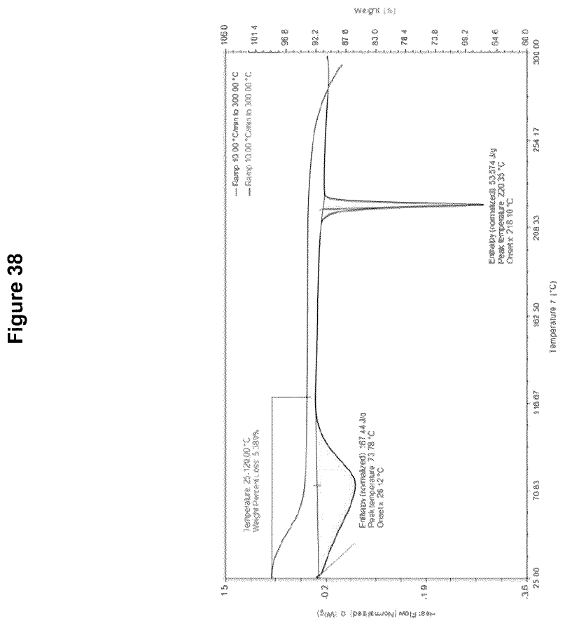

According to DSC, Form B shows one broad endothermic peak due to dehydration at 73.78° C. (onset 26.12° C.) and one melting peak at 220.35° C. (onset 218.10° C.). The TGA analysis of Form B shows a weight loss of about 5.39% from 25° C. to 120° C. (FIG. 38)

### Form C

Crystalline Form C is a dimesylate salt metastable form, THF solvate (0.5 eq).

Form C can be obtained from slurrying Compound 4, Form B in THF at room temperature and 50° C. for 3 days.

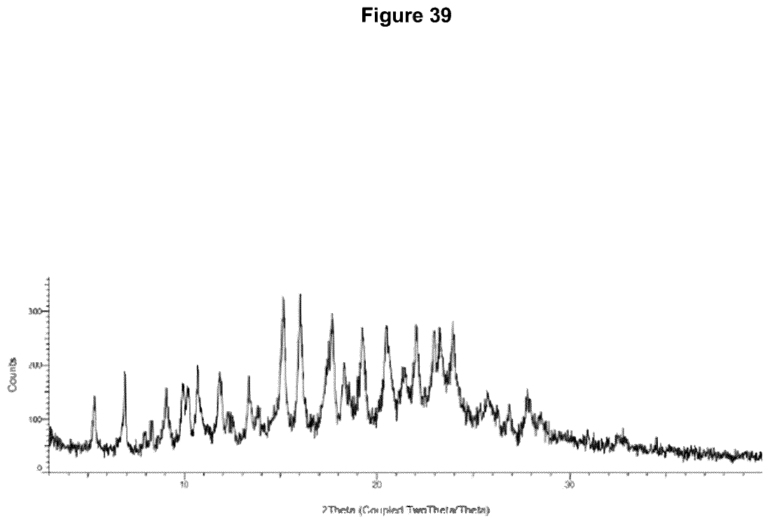

Form C exhibits an XRPD pattern (FIG. 39) having characteristic peaks expressed in degrees 2θ (±0.2° 2θ) at 15.2 and 16.0. The XRPD pattern of Form C can also exhibit further characteristic peaks expressed in degrees 2θ (±0.2° 2θ) at 17.6 and 23.0. The XRPD pattern of Form C can also exhibit further characteristic peaks expressed in degrees 2θ (±0.2° 2θ) at 20.5 and 23.3. The XRPD pattern of Form C can also exhibit further characteristic peaks expressed in degrees 2θ (±0.2° 2θ) at 23.9 and 19.3. The XRPD pattern of Form C can also exhibit further characteristic peaks expressed in degrees 2θ (±0.2° 2θ) at 22.1 and 6.9.

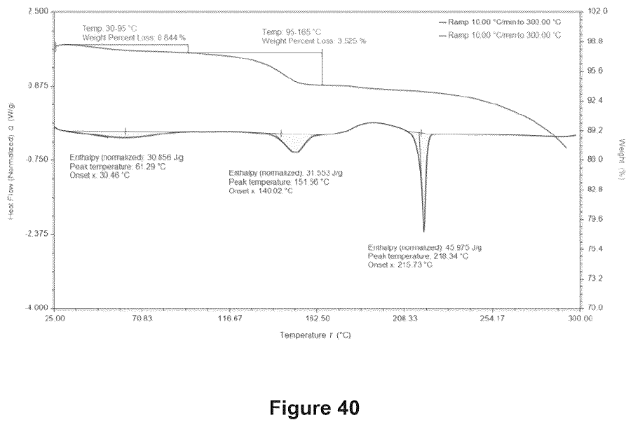

According to DSC, Form C shows three endothermic peaks at 61.29° C. (onset 30.46° C.), 151.56° C. (onset 140.02° C.) and at 218.34° C. (onset 215.73° C.). These three peaks might correspond to exclusion of free moisture, desolvation and melting. The TGA analysis of Form C shows a weight loss of about 0.8% and 3.5% at temperatures from 30° C. to 95° C. and 95° C. to 165° C. (FIG. 40)

### Form D

Crystalline Form D is a dimesylate salt monohydrate.

Form D can be obtained by various methods including slurrying, anti-solvent precipitation and reverse anti-solvent precipitation and are summarized in Example 7.

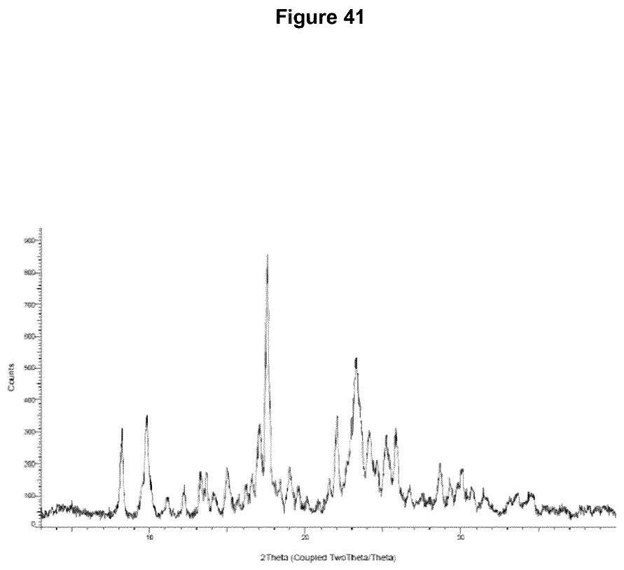

Form D exhibits an XRPD pattern (FIG. 41) having characteristic peaks expressed in degrees 2θ (±0.2° 2θ) at 17.6 and 23.2. The XRPD pattern of Form D can also exhibit further characteristic peaks expressed in degrees 2θ (±0.2° 2θ) at 22.0 and 8.2. The XRPD pattern of Form D can also exhibit further characteristic peaks expressed in degrees 2θ (±0.2° 2θ) at 9.8 and 25.9. The XRPD pattern of Form D can also exhibit further characteristic peaks expressed in degrees 2θ (±0.2° 2θ) at 17.1 and 24.1. The XRPD pattern of Form D can also exhibit further characteristic peaks expressed in degrees 2θ (±0.2° 2θ) at 25.2 and 15.0.

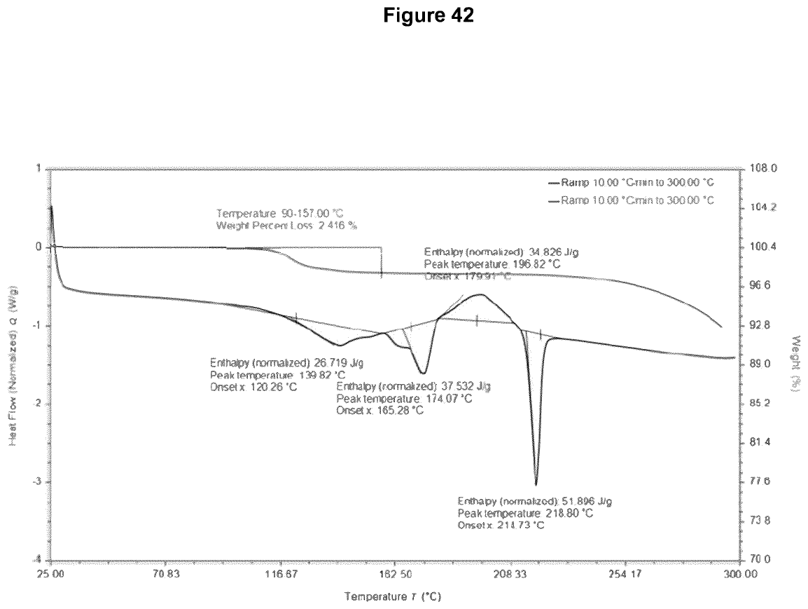

According to DSC, Form D shows three endothermic peaks at 139.82° C. (onset 120.26° C.), 174.07° C. (onset 165.28° C.) and at 218.80° C. (onset 214.73° C.), and one exothermic peak 196.82° C. (onset 179.91° C.). The first endothermic peak at 140° C. corresponded to dehydration. The second endothermic peak at 174° C. corresponded to melting. The followed exothermic peak ascribed to the form conversion from Form D to Form F. As the sample was heated up to elevated temperature, Form F melted at 219° C. The TGA analysis of Form D shows a weight loss of about 2.4% from 90° C. to 157° C. (FIG. 42)

### Form E

Crystalline Form E is a dimesylate salt unstable form.

Form E was obtained by cooling crystallization in NMP.

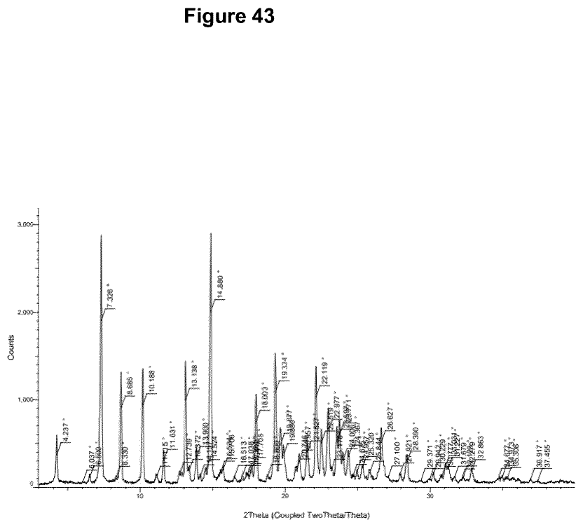

Form E exhibits an XRPD pattern (FIG. 43) having characteristic peaks expressed in degrees 2θ (±0.2° 2θ) at 14.9 and 7.3. The XRPD pattern of Form E can also exhibit further characteristic peaks expressed in degrees 2θ (±0.2° 2θ) at 19.3 and 13.1. The XRPD pattern of Form E can also exhibit further characteristic peaks expressed in degrees 2θ (±0.2° 2θ) at 10.2 and 8.7. The XRPD pattern of Form E can also exhibit further characteristic peaks expressed in degrees 2θ (±0.2° 2θ) at 22.1 and 18.0. The XRPD pattern of Form E can also exhibit further characteristic peaks expressed in degrees 2θ (±0.2° 2θ) at 23.0 and 23.8.

### Form F

Crystalline Form F is a dimesylate salt non-stoichiometric hydrate (1.5 eq at 30-50% RH).

Form F was obtained by heating Form D to 210° C.

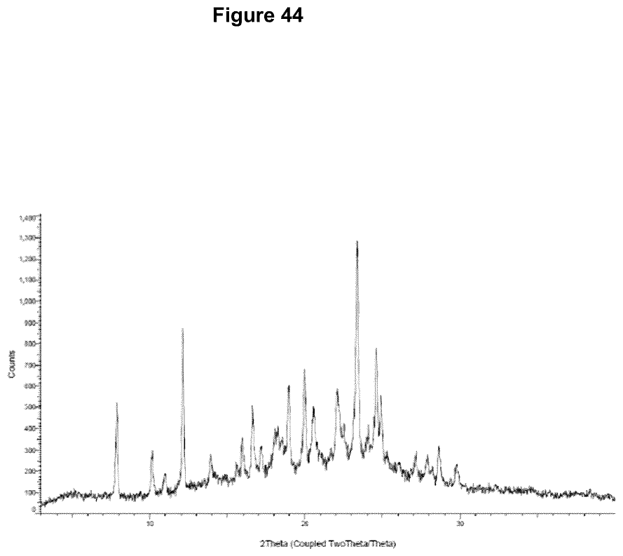

Form F exhibits an XRPD pattern (FIG. 44) having characteristic peaks expressed in degrees 2θ (±0.2° 2θ) at 23.4 and 12.1. The XRPD pattern of Form F can also exhibit further characteristic peaks expressed in degrees 2θ (±0.2° 2θ) at 24.6 and 20.0. The XRPD pattern of Form F can also exhibit further characteristic peaks expressed in degrees 2θ (±0.2° 2θ) at 7.9 and 24.9. The XRPD pattern of Form F can also exhibit further characteristic peaks expressed in degrees 2θ (±0.2° 2θ) at 19.0 and 16.6. The XRPD pattern of Form F can also exhibit further characteristic peaks expressed in degrees 2θ (±0.2° 2θ) at 20.6 and 22.1.

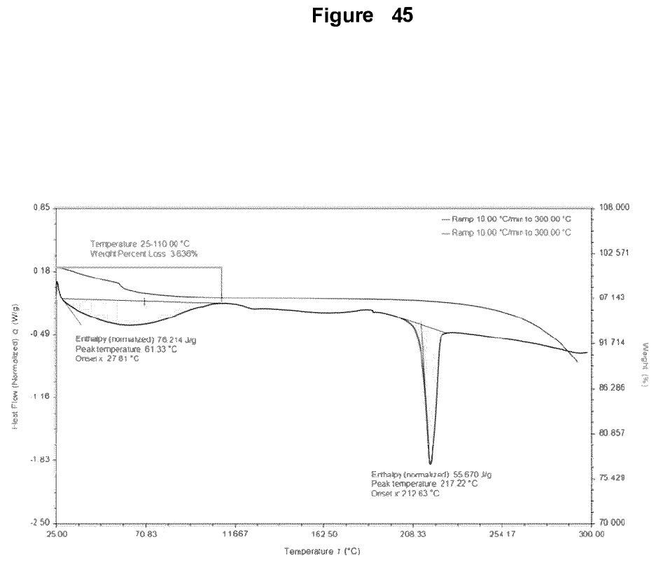

According to DSC, Form F shows two endothermic peaks at 61.33° C. (onset 27.81° C.), and at 217.22° C. (onset 212.63° C.). The first endothermic peak at 61.33° C. corresponds to dehydration. The second endothermic peak at 217° C. corresponds to melting. The TGA analysis of Form F shows a weight loss of about 3.6% from 25° C. to 110° C. (FIG. 45)

### Form G

Crystalline Form G is a mono-mesylate dihydrate.

Form G was obtained by slurrying Form B in water at 50° C.

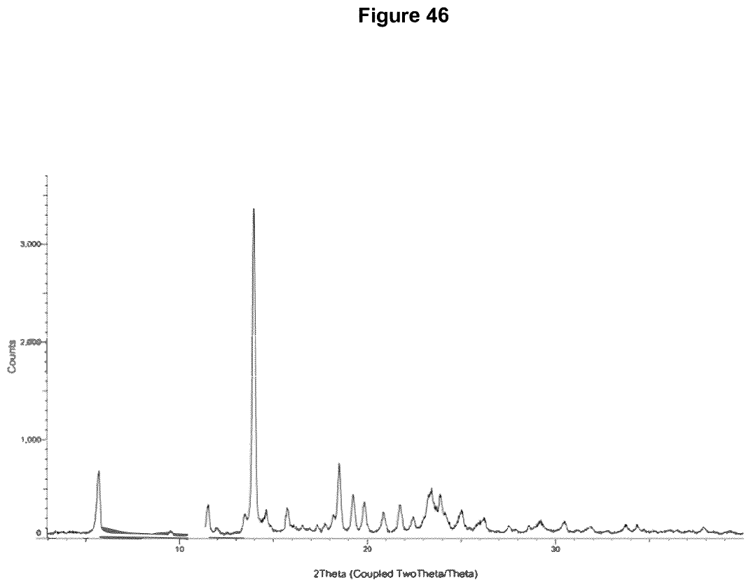

Form G exhibits an XRPD pattern (FIG. 46) having characteristic peaks expressed in degrees 2θ (±0.2° 2θ) at 14.0 and 18.5. The XRPD pattern of Form G can also exhibit further characteristic peaks expressed in degrees 2θ (±0.2° 2θ) at 5.7 and 19.2. The XRPD pattern of Form G can also exhibit further characteristic peaks expressed in degrees 2θ (±0.2° 2θ) at 23.3 and 23.9. The XRPD pattern of Form G can also exhibit further characteristic peaks expressed in degrees 2θ (±0.2° 2θ) at 19.8 and 11.5. The XRPD pattern of Form G can also exhibit further characteristic peaks expressed in degrees 2θ (±0.2° 2θ) at 21.7 and 15.7.

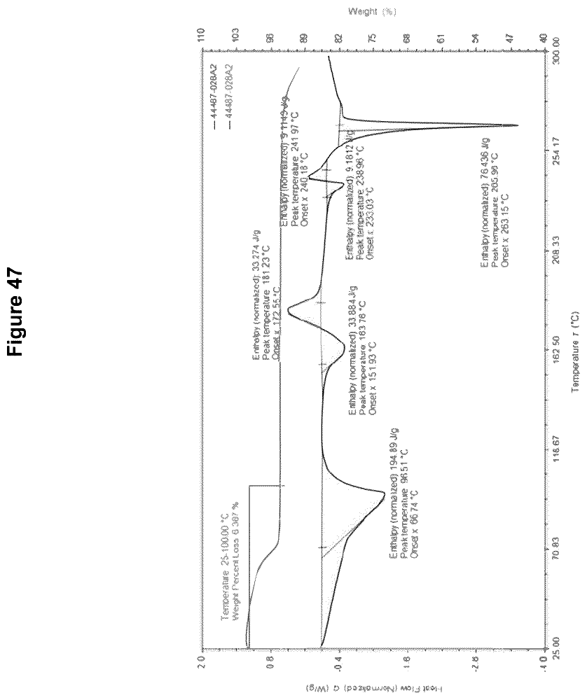

According to DSC, Form G shows four endothermic peaks at 96.51° C. (onset 66.74° C.), 163.76° C. (onset 151.93° C.), 238.96° C. (onset 233.03° C.) and at 265.96° C. (onset 263.15° C.), and two exothermic peaks at 181.23° C. (onset 172.55° C.) and 241.97° C. (onset 240.18° C.). The TGA analysis of Form G shows a weight loss of about 6.4% from 25° C. to 100° C. (FIG. 47)

### Other Salts

The salt screening experiments also include the attempted synthesis of the following salts of Compound 1: phosphate, L-tartrate, sulfonate, succinate, maleate, citrate and L-Lysine.

### Formulations, Methods and Uses

As used herein, the terms “effective amount” or “effective dose” mean that amount of a drug or pharmaceutical agent that will elicit the biological or medical response of a tissue, system, animal or human that is being sought, for instance, by a researcher or clinician. Furthermore, the terms “effective amount” or “effective dose” mean any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

The term “patient” or “subject” as used herein refers to a mammal. A subject therefore refers to, for example, dogs, cats, horses, cows, pigs, guinea pigs, and the like. Preferably the subject is a human. When the subject is a human, the subject may be either a patient or a healthy human.

The compounds of the present description can be formulated with conventional carriers and excipients, which will be selected in accordance with ordinary practice. Tablets will contain excipients, glidants, fillers, binders and the like. Aqueous formulations are prepared in sterile form, and when intended for delivery by other than oral administration generally will be isotonic. All formulations will optionally contain excipients such as those set forth in the Handbook of Pharmaceutical Excipients (1986), herein incorporated by reference in its entirety. Excipients include ascorbic acid and other antioxidants, chelating agents such as EDTA, carbohydrates such as dextrin, hydroxyalkylcellulose, hydroxyalkylmethylcellulose, stearic acid and the like. The pH of the formulations ranges from about 3 to about 11 but is ordinarily about 7 to 10.

While it is possible for the active ingredients to be administered alone it may be preferable to present them as pharmaceutical formulations. The formulations of the invention, both for veterinary and for human use, comprise at least one active ingredient, together with one or more acceptable carriers and optionally other therapeutic ingredients.

The carrier(s) must be “acceptable” in the sense of being compatible with the other ingredients of the formulation and physiologically innocuous to the recipient thereof.

The formulations include those suitable for the foregoing administration routes. The formulations may conveniently be presented in unit dosage form and may be prepared by any of the methods well known in the art of pharmacy. Techniques and formulations generally are found in Remington's Pharmaceutical Sciences (Mack Publishing Co., Easton, Pa.), herein incorporated by reference in its entirety. Such methods include the step of bringing into association the active ingredient with the carrier which constitutes one or more accessory ingredients. In general, the formulations are prepared by uniformly and intimately bringing into association the active ingredient with liquid carriers or finely divided solid carriers or both, and then, if necessary, shaping the product.

Formulations of the present invention suitable for oral administration may be presented as discrete units such as capsules, pastilles, cachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be administered as a bolus, electuary or paste.

A tablet is made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface active or dispersing agent.

Molded tablets may be made by molding in a suitable machine a mixture of the powdered active ingredient moistened with an inert liquid diluent. The tablets may optionally be coated or scored and optionally are formulated so as to provide slow or controlled release of the active ingredient.

Pharmaceutical formulations according to the present description include one or more compounds together with one or more pharmaceutically acceptable carriers or excipients and optionally other therapeutic agents. Pharmaceutical formulations containing the active ingredient may be in any form suitable for the intended method of administration. When used for oral use for example, tablets, pastilles, troches, lozenges, aqueous or oil suspensions, dispersible powders or granules, emulsions, hard or soft capsules, syrups or elixirs may be prepared. Compositions intended for oral use may be prepared according to any method known to the art for the manufacture of pharmaceutical compositions and such compositions may contain one or more agents including sweetening agents, flavoring agents, coloring agents and preserving agents, in order to provide a palatable preparation.

Tablets containing the active ingredient in admixture with non-toxic pharmaceutically acceptable excipient which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc. Tablets may be uncoated or may be coated by known techniques including microencapsulation to delay disintegration and adsorption in the gastrointestinal tract and thereby provide a sustained action over a longer period. For example, a time delay material such as glyceryl monostearate or glyceryl distearate alone or with a wax may be employed.

Formulations for oral use may be also presented as hard gelatin capsules where the active ingredient is mixed with an inert solid diluent, for example calcium phosphate or kaolin, or as soft gelatin capsules wherein the active ingredient is mixed with water or an oil medium, such as peanut oil, liquid paraffin or olive oil.

Aqueous suspensions of the invention contain the active materials in admixture with excipients suitable for the manufacture of aqueous suspensions. Such excipients include a suspending agent, such as sodium carboxymethylcellulose, methylcellulose, hydroxypropyl methylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethyleneoxycetanol), a condensation product of ethylene oxide with a partial ester derived from a fatty acid and a hexitol anhydride (e.g., polyoxyethylene sorbitan monooleate). The aqueous suspension may also contain one or more preservatives such as ethyl or n-propyl p-hydroxy-benzoate, one or more coloring agents, one or more flavoring agents and one or more sweetening agents, such as sucrose or saccharin.

Oil suspensions may be formulated by suspending the active ingredient in a vegetable oil, such as arachis oil, olive oil, sesame oil or coconut oil, or in a mineral oil such as liquid paraffin. The oral suspensions may contain a thickening agent, such as beeswax, hard paraffin or cetyl alcohol. Sweetening agents, such as those set forth herein, and flavoring agents may be added to provide a palatable oral preparation. These compositions may be preserved by the addition of an antioxidant such as ascorbic acid.

Dispersible powders and granules of the invention suitable for preparation of an aqueous suspension by the addition of water provide the active ingredient in admixture with a dispersing or wetting agent, a suspending agent, and one or more preservatives. Suitable dispersing or wetting agents and suspending agents are exemplified by those disclosed above. Additional excipients, for example sweetening, flavoring and coloring agents, may also be present.

The pharmaceutical compositions may also be in the form of oil-in-water emulsions. The oily phase may be a vegetable oil, such as olive oil or arachis oil, a mineral oil, such as liquid paraffin, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan monooleate, and condensation products of these partial esters with ethylene oxide, such as polyoxyethylene sorbitan monooleate. The emulsion may also contain sweetening and flavoring agents. Syrups and elixirs may be formulated with sweetening agents, such as glycerol, sorbitol or sucrose. Such formulations may also contain a demulcent, a preservative, a flavoring or a coloring agent.

The pharmaceutical compositions of the invention may be in the form of a sterile injectable preparation, such as a sterile injectable aqueous or oleaginous suspension. This suspension may be formulated according to the known art using those suitable dispersing or wetting agents and suspending agents which have been mentioned herein. The sterile injectable preparation may also be a sterile injectable solution or suspension in a non-toxic parenterally acceptable diluent or solvent, such as a solution in 1,3-butane-diol or prepared as a lyophilized powder. Among the acceptable vehicles and solvents that may be employed are water, Ringer's solution and isotonic sodium chloride solution. In addition, sterile fixed oils may conventionally be employed as a solvent or suspending medium. For this purpose, any bland fixed oil may be employed including synthetic mono- or diglycerides. In addition, fatty acids such as oleic acid may likewise be used in the preparation of injectables.

The amount of active ingredient that may be combined with the carrier material to produce a single dosage form will vary depending upon the host treated and the particular mode of administration. For example, a time-release formulation intended for oral administration to humans may contain approximately 1 to 1000 mg of active material compounded with an appropriate and convenient amount of carrier material which may vary from about 5 to about 95% of the total compositions (weight:weight). The pharmaceutical composition can be prepared to provide easily measurable amounts for administration. For example, an aqueous solution intended for intravenous infusion may contain from about 3 to 500 μg of the active ingredient per milliliter of solution in order that infusion of a suitable volume at a rate of about 30 mL/hr can occur.

Formulations for rectal administration may be presented as a suppository with a suitable base comprising for example cocoa butter or a salicylate.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

The formulations are presented in unit-dose or multi-dose containers, for example sealed ampoules and vials, and may be stored in a freeze-dried (lyophilized) condition requiring only the addition of the sterile liquid carrier, for example water for injection, immediately prior to use. Extemporaneous injection solutions and suspensions are prepared from sterile powders, granules and tablets of the kind previously described. Preferred unit dosage formulations are those containing a daily dose or unit daily sub-dose, as herein above recited, or an appropriate fraction thereof, of the active ingredient.

It should be understood that in addition to the ingredients particularly mentioned above the formulations of this invention may include other agents conventional in the art having regard to the type of formulation in question, for example those suitable for oral administration may include flavoring agents.

The compounds of the present description can also be formulated to provide controlled release of the active ingredient to allow less frequent dosing or to improve the pharmacokinetic or toxicity profile of the active ingredient. Accordingly, there is also provided compositions comprising one or more compounds of the present description formulated for sustained or controlled release.

The effective dose of an active ingredient depends at least on the nature of the condition being treated, toxicity, whether the compound is being used prophylactically (lower doses) or against an active disease or condition, the method of delivery, and the pharmaceutical formulation, and will be determined by the clinician using conventional dose escalation studies. The effective dose can be expected to be from about 0.0001 to about 10 mg/kg body weight per day, typically from about 0.001 to about 1 mg/kg body weight per day, more typically from about 0.01 to about 1 mg/kg body weight per day, even more typically from about 0.05 to about 0.5 mg/kg body weight per day. For example, the daily candidate dose for an adult human of approximately 70 kg body weight will range from about 0.05 mg to about 100 mg, or between about 0.1 mg and about 25 mg, or between about 0.4 mg and about 4 mg, and may take the form of single or multiple doses.

The present description relates to compounds or pharmaceutically acceptable salts thereof, for the treatment various conditions treatable by inhibiting SGK-1. For example, the condition can be Long QT syndrome (LQTS), such as genetic LQTS or acquired LQTS, or other cardiovascular diseases (e.g., dilated cardiomyopathy—genetic or acquired) that are treatable by inhibiting SGK-1. Without being bound by theory, it is believed that SGK-1 inhibition in vivo has a protective effect and can alleviate symptoms associated with LQTS; can reduce and alleviate symptoms associated with heart failure, arrhythmia such as atrial fibrillation, ischemic injury, ischemic infarction, cardiac fibrosis, vascular proliferation, restenosis, genetic or acquired dilated cardiomyopathy, hypertrophic cardiomyopathy, and stent failure.

Long QT syndrome (LQTS) can be genetic (e.g. caused by a mutation in the KCNQ1 gene, the KCNH2 gene, or the SCN5a gene). Alternatively, Long QT syndrome is not associated with a genetic mutation and is acquired as a result of exposure to an external stimulus. For instance, acquired Long QT syndrome can be a side effect of drugs such as erythromycin or haloperidol. Acquired Long QT syndrome is also associated with other heart conditions such as myocardial ischemia.

The present description also relates to compounds or pharmaceutically acceptable salts thereof, for the treatment of other conditions related to SGK-1 mediated mechanisms, such as cancer, Parkinson's disease and Lafora disease.

In some embodiments, the present description provides compounds or pharmaceutically acceptable salts thereof for treating cancer or another proliferative disorder. As used herein, the terms “inhibition of cancer”, “inhibition of cancer cell proliferation”, and “inhibition of cancer invasion and metastasis” refer to the inhibition, or decrease in the rate, of the growth, division, maturation, viability, or ability to invade and colonize other organs and tissues of cancer cells, and/or causing the death of cancer cells, individually or in aggregate with other cancer cells, by cytotoxicity, nutrient depletion, induction of differentiation or apoptosis, or recognition by the immune system in order to elicit an immune response to the cancer cells.

Examples of tissues containing cancerous cells whose proliferation can be inhibited by a compound, salt or composition thereof described herein and against which the methods described herein are useful include but are not limited to breast, prostate, brain, blood, bone marrow, liver, pancreas, skin, kidney, colon, intestine, endometrium, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, bone, and stomach.

In some embodiments, the cancer treated by a provided compound, salt or composition thereof is a melanoma, liposarcoma, lung cancer, breast cancer, prostate cancer, leukemia, kidney cancer, esophageal cancer, brain cancer, lymphoma, colon cancer or colorectal cancer. In some embodiments, the cancer treated by a provided compound, salt or composition thereof is prostate cancer, colorectal cancer or breast cancer (e.g., resistant breast cancer).

In some embodiments, the compounds of the present description can be used to treat cancer by inhibiting signaling of the AKT/PI3K/mTOR pathway in patients whose tumors have activation of this pathway through mutations in PIK3CA, AKT1, and/or PTEN for example.

In some embodiments, the compounds of the present description can be used in combination with compounds that inhibit AKT/PI3K/mTOR signaling to treat cancer in patients whose tumors have activation of this pathway through mutations in PIK3CA, AKT1, and/or PTEN for example. Non-limiting examples of AKT/PI3K/mTOR inhibitors include NVP-BEZ235 (BEZ235, Dactolisib), GDC-0084 (RG7666), GDC-0980 (Apitolisib, RG7422), LY3023414, PF-05212384 (Gedatolisib, PKI-587), PQR309 (Bimiralisib), P7170, SF-1126, Copanlisib (BAY 80-6946), Buparlisib (BKM120 NVP-BKM120), IPI-145 (Duvelisib), RP6530 (Tenalisib), GDC-0032 (Taselisib), KA2237, BYL719 (Alpelisib), CAL-101 (GS-1101, Idelalisib), GSK2636771, INCB050465 (Parsaclisib), Serabelisib (INK-1117, MLN-1117, TAK-117), ME401 (PWT-143), Umbralisib (RP5264, TGR-1202), CUDC-907 (Fimepinostat), Rigosertib (ON-01910), samotolisib, paxalisib, voxtalisib, CH5132799, pilaralisib, ZSTK474, sonolisib, pictilisib, B591, TG-100-115, RIDR-PI-103, zandelisib, AMG319, linperlisib, leniolisib, eganelisib, AZD8186, AZD8835, MK-2206, ipatasertib, GSK690693, capivasertib, PF-04691502, AT7867, MAY1125976, TAS117, Afuresertib, Uprosertib, AT13148, everolimus, temsirolimus, ridaforolimus, sirolimus, umirolimus, zotarolimus, ICSN3250, LY3023414, OSU-53, AZD8055, and rapamycin.

In some embodiments, the compounds of the present description can be used to treat inflammatory and fibrotic diseases that can include fatty liver diseases, endometriosis, types 1 or 2 diabetes mellitus, inflammatory bowel disease, asthma, rheumatoid arthritis, obesity, systemic sclerosis, sclerodermatous graft vs. host disease, nephrogenic systemic fibrosis, as well as organ-specific fibrosis, including radiation-induced fibrosis, and auto-immune diseases.

Serine/threonine-protein kinase (SGK-1) (also known as serum/glucocorticoid-regulated kinase 1) is a protein kinase that plays a role in a cell's response to stress. In vivo, SGK-1 activates certain potassium, sodium, and chloride channels. For instance, the protein is known to regulate the myo-inositol transporter during osmotic stress. The term “inhibitor of SGK-1”, as used herein, refers to any compound that can block, arrest, interfere with, or reduce the biological activity of SGK-1.

In some embodiments, the compounds of the present description can be used for increasing fetal hemoglobin (HbF) in erythrocytes. In some embodiments, the compounds of the present description can be used for the treatment of a β-hemoglobinopathy. In some embodiments, the compounds of the present description can be used for the treatment of sickle cell disease.

In some embodiments, the compounds of the present description can be used for the treatment of prostate cancer. In other embodiments, the compounds of the present description can be used for the treatment of epilepsy.

The compounds of the present description and their pharmaceutically acceptable salts thereof are pharmacologically active compounds that modulate protein kinase activity, specifically the activity of serum and glucocorticoid regulated kinase isoform 1 (SGK-1).

The compounds of the present description or their pharmaceutically acceptable salts can be suitable for the treatment of conditions in which SGK-1 activity is inappropriate. Non-limiting examples of such conditions can include Long QT syndrome, heart failure, arrhythmia such as atrial fibrillation, ischemic injury, ischemic infarction, cardiac fibrosis, vascular proliferation, restenosis, dilated cardiomyopathy, stent failure, prostate cancer and epilepsy. Other non-limiting examples of such conditions include β-hemoglobinopathies, such as sickle cell disease.

### EXPERIMENTS AND EXAMPLES

### Materials and Instruments

Selected XRPD patterns were collected with a PANalytical X'Pert PRO MPD or a PANalytical Empyrean diffractometer using an incident beam of Cu radiation produced by an Optix long, fine-focus source. An elliptically graded multilayer mirror was used to focus Cu Kα. X-rays through the specimen and onto the detector. Prior to the analysis, a silicon specimen (NIST SRM 640f was analyzed to verify the Si 111 peak position. A specimen of the sample was sandwiched between 3 μm thick films and analyzed in transmission geometry. A beam-stop and short antiscatter extension were used to minimize the background generated by air. Soller slits for the incident and diffracted beams were used to minimize broadening from axial divergence. Diffraction patterns were collected using a scanning position-sensitive detector (X'Celerator) located 240 mm from the specimen and Data Collector software v.5.5.

| X-Ray wavelength | 1.5405929 Å |
|---|---|
| Divergence slit | ½° |
| Scan range (°2TH) | ~1-40° |
| Step size (°2TH) | 0.0167 |
| Time/Step | 36.830 |

XRPD patterns were also collected with a PANalytical X'Pert PRO MPD diffractometer using an incident beam of Cu Kα radiation produced using a long, fine-focus source and a nickel filter. The diffractometer was configured using the symmetric Bragg-Brentano geometry. Prior to the analysis, a silicon specimen (NIST SRM 640f was analyzed to verify the observed position of the Si 111 peak is consistent with the NIST-certified position. A specimen of the sample was prepared as a thin, circular layer centered on a silicon zero-background substrate. Antiscatter slits (SS) were used to minimize the background generated by air. Soller slits for the incident and diffracted beams were used to minimize broadening from axial divergence. Diffraction patterns were collected using a scanning position-sensitive detector (X'Celerator) located 240 mm from the sample and Data Collector software v. 2.2b. The data acquisition parameters for each pattern are displayed above the image in the Data section of this report including the divergence slit (DS) and the incident-beam SS.

| X-Ray wavelength | 1.54059 Å |
|---|---|
| Divergence slit | ⅛° |
| Scan range (°2TH) | 3.51-39.99° |
| Step size (°2TH) | 0.017 |
| Time/Step | 36.830 |
| Scan Speed (°min) | 1.2 |
| Collection Time (s) | 1828 |

The XRPD pattern were indexed using X'Pert High-Score Plus 2.2a (2.2.1), TOPAS or proprietary software.

Differential Scanning Calorimetry (DSC) and ThermoGravimetric Analysis (TGA) were performed on a Mettler-Toledo TGA/DSC3+ analyzer. Temperature and enthalpy adjustments were performed using indium, tin, and zinc, and then verified with indium. The balance was verified with calcium oxalate. The sample was placed in an open aluminum pan. The pan was hermetically sealed, the lid pierced, then inserted into the TG furnace. A weighed aluminum pan configured as the sample pan was placed on the reference platform. The furnace was heated under nitrogen. Data was collected from 25° C. to 350° C. at 10° C./min.

Other XRPD diffractograms were collected with an X-ray diffractometer. The sample was prepared on a zero-background silicon wafer by gently pressing onto the flat surface. The parameters of XRPD diffraction are the following:

| | |
|---|---|
| Instrument | Bruker, D2 Advance |
| Radiation | Cu Kα (λ = 1.5418 Å) |
| Detector | LynxEye |
| Scan angle | 3-40° (2θ) |
| Scan step | 0.02° (2θ) |
| Scan speed | 0.2 s/step |
| Tube voltage/current | 30 kV/10 mA |
| Divergence slit | 0.6 mm |
| Rotation | On |
| Sample holder | Zero-background sample pan |

Other TGA analysis were performed using a TA instrument. Data was analyzed using TRIOS. About 1-5 mg of sample was loaded onto a pre-tared aluminium pan and heated with the following parameters:

| | |
|---|---|
| Instrument | TA, Discovery TGA 55 |
| Sample pan | Aluminium, open |
| Temperature range | RT-300° C./350° C. |
| Heating rate | 10° C./min |
| Purge gas | N₂ |
| Flow rate | Balance chamber: 40 mL/min |
| | Sample chamber: 25 mL/min |

Other DSC analysis were performed using a TA instrument. Data was analyzed using TRIOS. About 1-3 mg of sample was loaded onto an aluminium pan with a pin-hole and heated with the following parameters:

| | |
|---|---|
| Instrument | TA, Discovery DSC 250 |
| Sample pan | Aluminium, pin-holed |
| Temperature range | 25-300° C./350° C. |
| Heating rate | 10° C./min |
| Purge gas | N₂ |
| Flow rate | 50 mL/min |

Moisture sorption/desorption data were collected on a DVS Intrinsic. 7-20 mg of a sample was placed into a tared sample chamber and automatically weighed. The anhydrates were analyzed with the following setting parameters:

| Instrument | SMS, DVS Intrinsic |
|---|---|
| dm/dt | 0.002%/min |
| Sample size | 10-30 mg |
| Drying/Measurement temperature | 40° C./25° C. |
| Cycle | Full cycle |
| Minimum dm/dt | 30 min |
| stability duration | |
| Maximum dm/dt | 120 min |
| equilibrium time | |
| Save data rate | 5 s |
| Gas and Total flow rate | N₂, 200 sccm |
| Post experiment total flow | 10% RH |
| RH step size, | Adsorption: 0, 10, 20, 30, 40, |
| Relative Humidity | 50, 60, 70, 80, 90 |
| Method | Desorption: 80, 70, 60, 50, 40, |
| | 30, 20, 10, 0 |

Some of the solution nuclear magnetic resonance spectra were acquired with an Avance 600 MHz NMR Spectrometer. Other ¹H-NMR spectra were collected on a Bruker 400 MHz instrument. The samples were prepared by dissolving approximately 4-7 mg of sample in dimethylsulfoxide-d6. Data were analyzed using MestReNova.

The solvent abbreviations are as follows: MeOH: Methanol; THF: tetrahydrofuran; EtOH: Ethanol; IPAC: Isopropyl acetate; EtOAc or EA: Ethyl acetate; DMF: Dimethylformamide; IPA: Isopropanol; DCM: dichloromethane; DMSO: Dimethylsulfoxide; ACN: Acetonitrile; MTBE: Methyl tert-butyl ether; EA: Ethyl amine; NMP: N-methyl-2-pyrrolidone; DMA: Dimethylformamide; MEK: Methylethylketone; MTBE: Methyl tert-butyl ether; MIBK: Methylisobutylketone; IPE: Isopropyl ether.

HPLC analysis were performed with an Agilent HPLC 1260 series instrument. HPLC method for stability testing is as follows:

| Instrument | Agilent 1260 series | | |
|---|---|---|---|
| Column | Agilent Poroshell 120 EC-C18, 2.7 μm, 4.6 × 100 mm | | |
| Column | 40° C. | | |
| temperature | | | |
| Mobile phase | A: 5 mM CH₃COONH₄ in water | | |
| | (pH = 8.8 ± 0.05)/B: ACN | | |
| Flow rate | 1.0 mL/min | | |
| Injection volume | 6 μL | | |
| Detector and | DAD; 220 nm | | |
| Wavelength | | | |
| Run time | 20 min | | |
| Post time | 2.0 min | | |
| Diluent | MeOH:water (v:v 1:1) | | |
| | Time (min) | % A | % B |
| Gradient | 0 | 90.0 | 10.0 |
| | 15.0 | 50.0 | 50.0 |
| | 20.0 | 5.0 | 95.0 |
| | 20.10 | 90.0 | 10.0 |

### Summary of the Experimental Results

Salt formation experiments were conducted with Compound 1 (N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide).

The resulting pharmaceutically acceptable salts, isolated as crystalline compounds, were characterized by various techniques (XRPD, DSC, TGA, NMR) and their stability investigated (DVS, slurry experiments).

### Example 1: Synthesis of Compound 1

### N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide

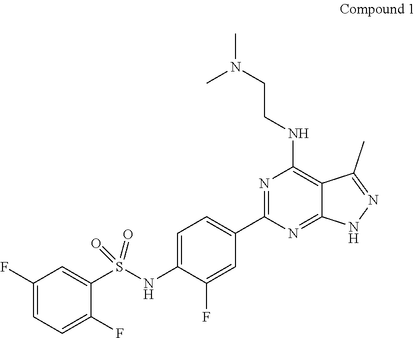 [SMILES: Cc1n[nH]c2nc(-c3ccc(NS(=O)(=O)c4cc(F)ccc4F)c(F)c3)nc(NCCN(C)C)c12]

### (i) 6-chloro-N-[2-(dimethylamino)ethyl]-3-methyl-1-(oxan-2-yl)pyrazolo[3,4-d]pyrimidin-4-amine

A 8-mL vial was charged with 4,6-dichloro-3-methyl-1-(oxan-2-yl)-2H,3H-pyrazolo[3,4-d]pyrimidine (74.3 mg, 0.259 mmol, 1 equiv.), (2-aminoethyl)dimethylamine (22.8 mg, 0.259 mmol, 1 equiv.), dichloromethane (3.0 mL) and triethylamine (57.7 mg, 0.571 mmol, 2.2 equiv.). The resulting solution was stirred overnight at room temperature and concentrated under reduced pressure. The residue was purified by silica gel column chromatography, eluted with dichloromethane/methanol (95/5) to afford desired product 6-chloro-N-[2-(dimethylamino)ethyl]-3-methyl-1-(oxan-2-yl)pyrazolo[3,4-d]pyrimidin-4-amine (55.0 mg, 62% yield) as a colorless solid.

### (ii) N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide

A 8-mL vial was charged with 2,5-difluoro-N-[4-(4,4,5,5-tetramethyl-1,3,2-dioxaborolan-2-yl)-2-fluorophenyl]benzenesulfonamide (1 equiv.), 6-chloro-N-[2-(dimethylamino)ethyl]-3-methyl-1-(oxan-2-yl)pyrazolo[3,4-d]pyrimidin-4-amine (1 equiv.), 1,4-dioxane/water (10:1), cesium carbonate (2 equiv.), [1,1′-bis(diphenylphosphino) ferrocene]dichloropalladium (0.1 equiv.). The resulting solution was stirred overnight at 100° C. The solids were filtered off and the filtrate was concentrated under reduced pressure. The crude product was purified by reverse phase column chromatography with the following conditions: Column, Column: Agela C18 Column, 120 g, Mobile Phase A: Water (0.05% TFA), Mobile Phase B: ACN; Flow rate: 40 mL/min; Gradient: 0% B to 55% B in 45 min; Detector: 220 nm to afford desired product N-[4-(4-[[2-(dimethylamino)ethyl]amino]-3-methyl-1-(oxan-2-yl)pyrazolo[3,4-d]pyrimidin-6-yl)-2-fluorophenyl]-2,5-fluorobenzenesulfonamide (51% yield) as a brown solid.

A 25-mL 2-necked round-bottom flask was charged with N-[4-(4-[[2-(dimethylamino)ethyl]amino]-3-methyl-1-(oxan-2-yl)pyrazolo[3,4-d]pyrimidin-6-yl)-2-fluorophenyl]-2,5-difluorobenzenesulfonamide (1 equiv.), isopropanol, 2M hydrochloric acid (gas) in 1,4-dioxane. The resulting solution was stirred for 3 h at room temperature and concentrated under reduced pressure. The crude product was purified by Prep-HPLC with the following conditions: Column: Xselect CSH OBD Column 30×150 mm, 5 um. Mobile Phase A: Water (10 mmol/L NH₄HCO₃+0.1% NH₃·H₂O), Mobile Phase B: ACN; Flow rate: 60 mL/min; Gradient: 18% B to 38% B in 7 min; Detector: 220 nm to afford desired product as a white solid (7.84% yield). LCMS (ES. m/z): 522 [M-TFA+H]+. ¹H-NMR (CD₃OD, 300 MHz) δ (ppm): 8.19-8.16 (m, 1H), 8.10-8.06 (dd, J=1.8 &12 Hz, 1H), 7.81-7.78 (m, 1H), 7.68-7.63 (m, 2H), 7.57 (t, J=8.2 Hz, 1H), 7.35-7.29 (t, J=9.3 Hz, 1H), 4.16-4.13 (t, J=5.7 Hz, 2H), 3.56-3.53 (t, J=5.7 Hz, 2H), 2.98 (s, 6H), 2.65 (s, 3H).

Compound 1 was further characterized having pKa values of 2.7, 5.81, 8.85 and 11.84.

| pH | <2.73 | 2.73-5.81 |
|---|---|---|
| Protonation state (Dominant form) | 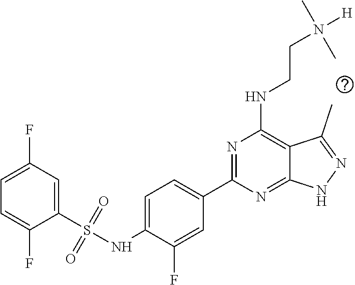 | 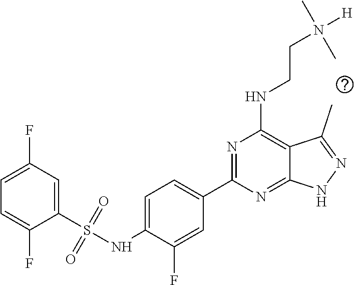 |
| Charge | 2+ | + |
| pH | 5.81-8.85 | 8.85-11.84 |
| Protonation state (Dominant form) | 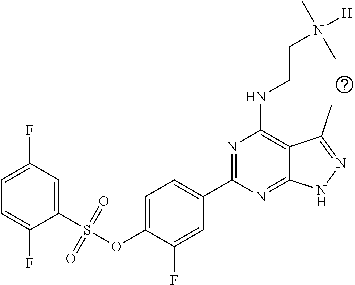 | 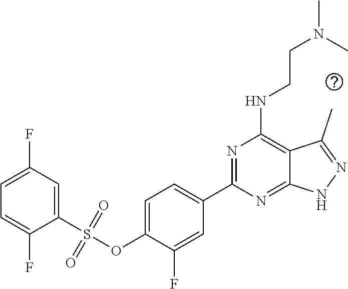 [SMILES: Cc1n[nH]c2nc(-c3ccc(OS(=O)(=O)c4cc(F)ccc4F)c(F)c3)nc(NCCN(C)C)c12] |
| Charge | Neutral (Zwitterion) | − |

| pH | >11.84 |
|---|---|
| Protonation state (Dominant form) | 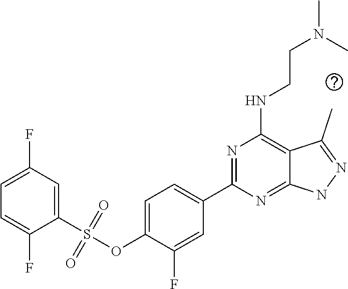 [SMILES: Cc1n[nH]c2nc(-c3ccc(OS(=O)(=O)c4cc(F)ccc4F)c(F)c3)nc(NCCN(C)C)c12] |
| Charge | 2− |

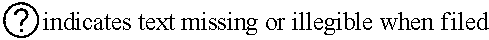

### Example 2: Solubility Screening of Compound 1

Solubility estimates were conducted using an aliquot addition method in a variety of solvents at ambient temperature. Poor solubility was observed in most solvents at ambient temperature. Immediate solubility was observed in DMF and DMSO.

TABLE 1
Solubility assays for compound 1 in various solvents (mg/mL)

| Solvent | Solubility at RT | Solubility at 50° C. |
|---|---|---|
| Acetone | <1 | <1 |
| ACN | <1 | <1 |
| DCM | <1 | <1 |
| DMF | 33 | — |
| DMSO | 41 | — |
| EtOAc | <3 | <3 |
| IPA | <2 | <2 |
| EtOH | <2 | <2 |
| MeOH | <1 | >1 |
| MTBE | <2 | <2 |
| THF | <1 | >1 |
| Water | <2 | <2 |

Samples with solvents with poor solubility were heated at 50° C. Dissolution was observed in methanol and THF.

### Example 3: Crystalline Forms of Compound 1 (Freebase)

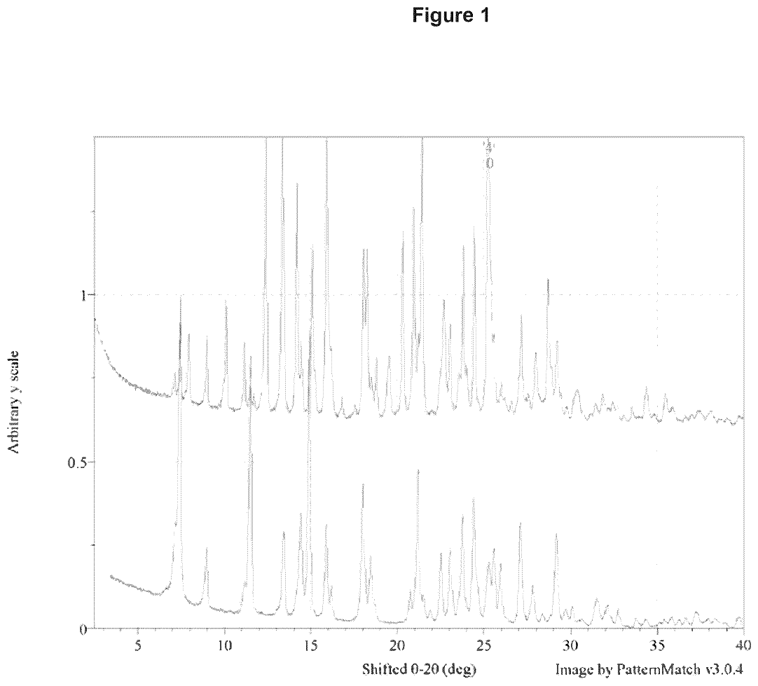

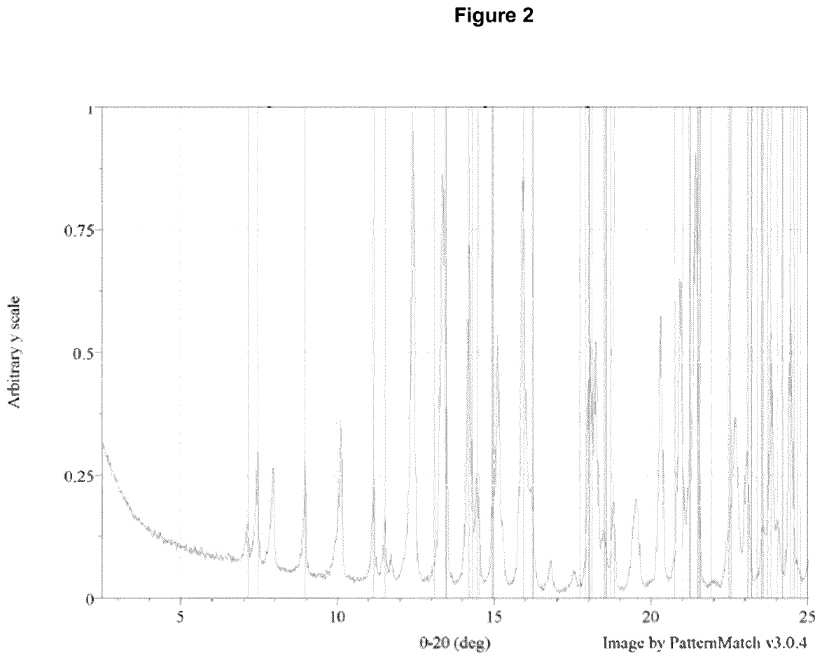

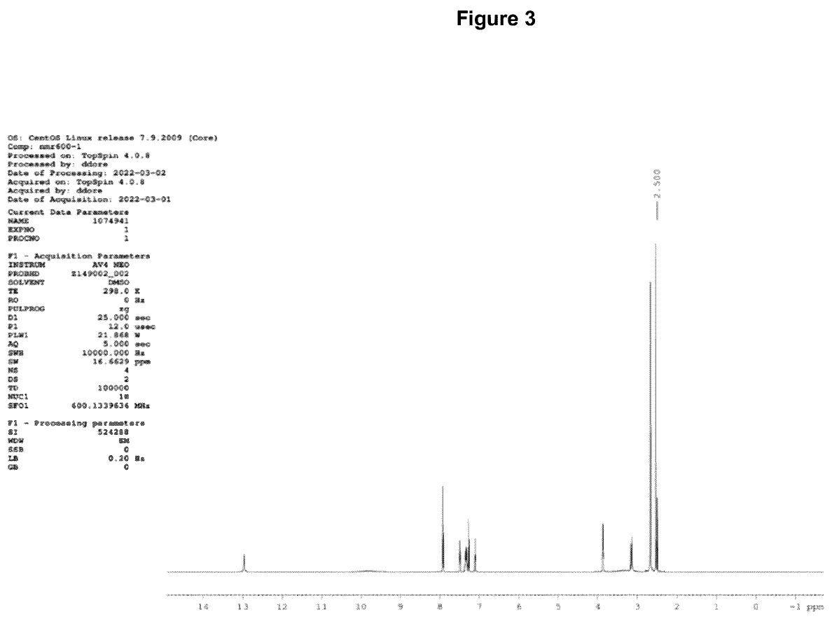

Compound 1 was characterized as a crystalline material composed of Material A and Form B, as displayed in FIG. 1 (XRPD pattern) and FIG. 2 (expanded XRPD pattern). The compound was also characterized by proton NMR (FIG. 3).

Free Form Form C was identified as the MeOH solvate of Compound 1. XRPD pattern is displayed in FIG. 12.

Free Form D can be obtained from NMP/Water and DMA/Water by anti-solvent precipitation, and in DMSO/Water by reverse anti-solvent precipitation. The starting material used to obtain Free Form D is HCl salt (Compound 3) Form B.

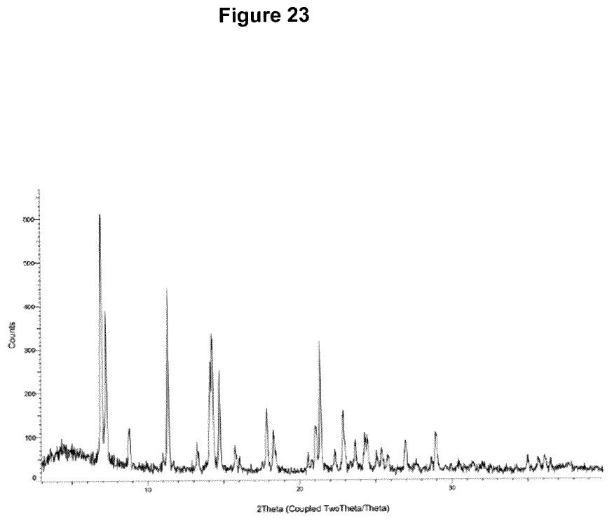

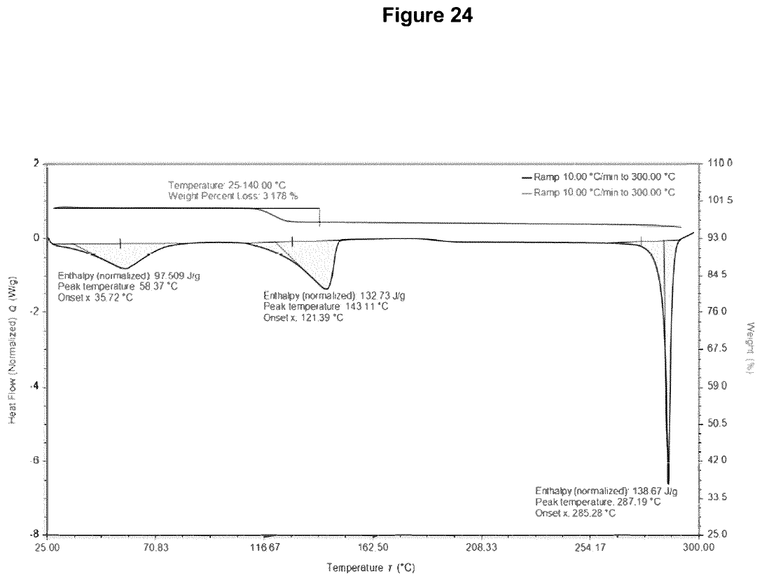

150 mg of Compound 3 (Form B) was dissolved in 7 mL of DMA at 70° C. and then the solution was filtered at room temperature. 84 mL of water (anti-solvent) was charged in the filtrate in 10 hours. The suspension was stirred at room temperature for 4 days. Free Form D was recovered by filtration and vacuum dried at 50° C. for 3 hours. The recovery yield is 13.8%. The characterization data of Free Form D are given in FIGS. 23-24. The sample was long bar shaped crystals. There was no residual solvents detected by ¹H-NMR. Moreover, a chemical shift in ¹H-NMR spectrum was observed compared with HCl salt, indicated the HCl salt was dissociated to the Free Form. TG analysis of Free Form D indicated 3.2% (1 eq. of water) of weight loss at 25-140° C. attributed to dehydration. The first and second endothermic peaks 58.37° C. (onset 35.72° C.) and 143.11° C. (onset 121.39° C.) ascribed to exclusion of absorbed moisture and dehydration, respectively. Free Form D was converted to Form E after dehydration, and thus the sharp endothermic peak at 287.19° C. (onset 285.28° C.) corresponds to the melting of Form E. Free Form D is a monohydrate with modest crystallinity. Free Form E is an anhydrate with high crystallinity.

### Example 4: Preparation of the Formate Salt

The formate salt was obtained via the following synthesis steps:

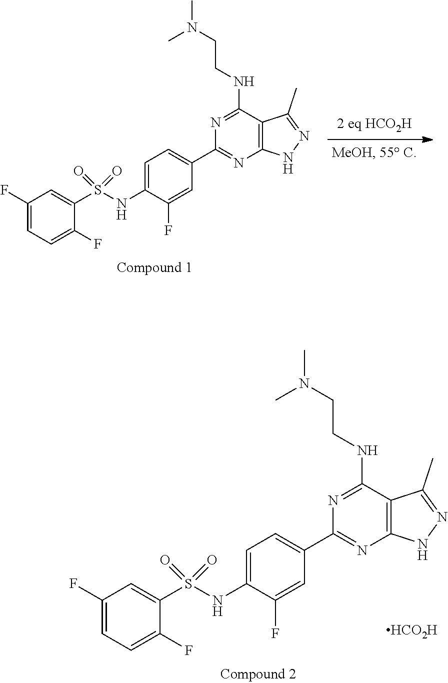 [SMILES: Cc1n[nH]c2nc(-c3ccc(NS(=O)(=O)c4cc(F)ccc4F)c(F)c3)nc(NCCN(C)C)c12 ; Cc1n[nH]c2nc(-c3ccc(NS(=O)(=O)c4cc(F)ccc4F)c(F)c3)nc(NCCN(C)C)c12 ; "·HCO2H"]

1) Stir Compound 1 (92 mg) in methanol (1 mL) at 55° C. to obtain a slurry. 2) Add 2 molar equivalents of formic acid (14 μL). Most solids are dissolved, a thin slurry is obtained.
3) Cool the solution to room temperature at a rate of 6° C. per hour and stir at room temperature for 3 days.
4) Using a Swinnex filter assembly, isolate solids for XRPD analysis.

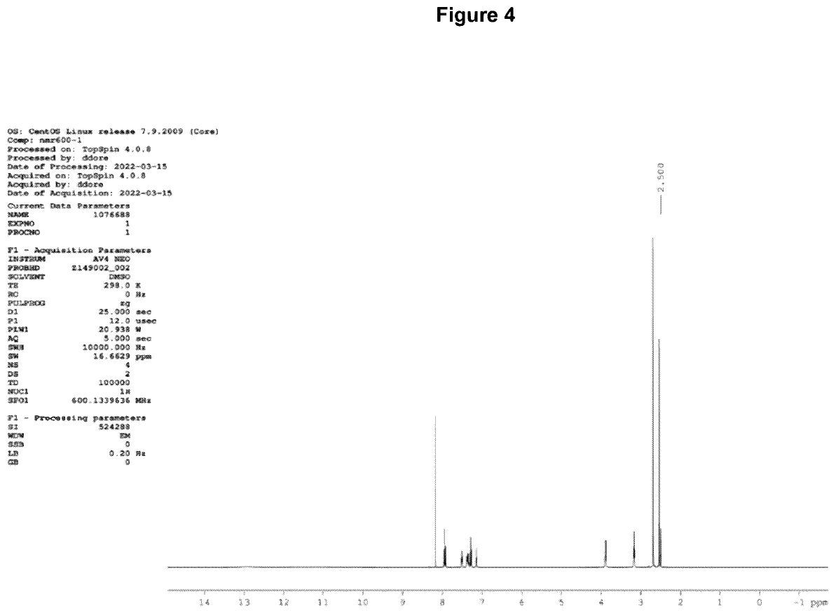

Compound 2 was obtained as shown by proton NMR (FIG. 4).

XRPD pattern analysis (FIGS. 5a and 5b) and indexing of the pattern (FIG. 6) indicated the formation of the crystalline anhydrous mono-formate salt (Form A) of Compound 1.

Thermal analysis (TGA) conducted on the salt showed a weight loss of 0.1% from 51 to 141° C. and a weight loss of 8.2% from 140 to 228° C. (˜1 mol formic acid). DSC analysis also revealed a broad endothermic peak (maxima) at 211° C. with an onset of 188° C. and an endothermic peak (maxima) at 289° C. with an onset of 287° C. as displayed in FIG. 7.

Solubility in water: <1 mg/mL.

### Example 5: Preparation of the Hydrochloride Salt and Crystalline Forms Thereof

The hydrochloride salt was obtained via the following experimental procedures:

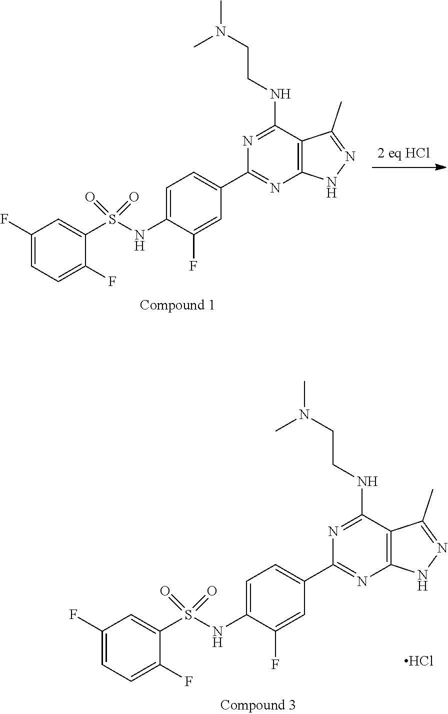 [SMILES: Cc1n[nH]c2nc(-c3ccc(NS(=O)(=O)c4cc(F)ccc4F)c(F)c3)nc(NCCN(C)C)c12 ; Cc1n[nH]c2nc(-c3ccc(NS(=O)(=O)c4cc(F)ccc4F)c(F)c3)nc(NCCN(C)C)c12 ; Cl]

### Form A

### Experimental Procedure a:

1) Stir Compound 1 (94 mg) in methanol (0.5 mL) at 55° C. to obtain a slurry. 2) Add 2 molar equivalents of hydrochloric acid (30 μL of 37%), an immobile slurry is obtained.
3) Add 2.5 mL of methanol, a mobile slurry is obtained. The solution was stirred at 55° C. for 3 days.
4) Using a Swinnex filter assembly, isolate solids for XRPD analysis.

XRPD pattern analysis indicated the formation of a mixture of Compound 3 as a unique crystalline material (Form A) and minor unknown XRPD peaks.
5) Take the previously isolated solid in acetone to form a slurry.
6) Stir at RT for 2 days.
7) Using a Swinnex filter assembly, isolate solids for XRPD analysis.

XRPD pattern analysis indicated the formation of a mixture of Compound 3 as a unique crystalline material (Form A) and minor unidentified XRPD peaks.
or

### Experimental Procedure B:

1) Stir Compound 1 (98 mg) in methanol (1 mL) at RT to obtain a slurry.
2) Add 2 molar equivalents of hydrochloric acid (32 μL of 37%), an immobile slurry is obtained.
3) Stir at 60° C., an immobile slurry is obtained.
4) Add water (0.2 mL), a mobile slurry is obtained.
5) Mix on rotating wheel at RT for 12 days.
6) Using a Swinnex filter assembly, isolate solids for XRPD analysis.

XRPD pattern analysis indicated the formation of a mixture of Compound 3 as a unique crystalline material (Form A) and minor additional unknown XRPD peaks.

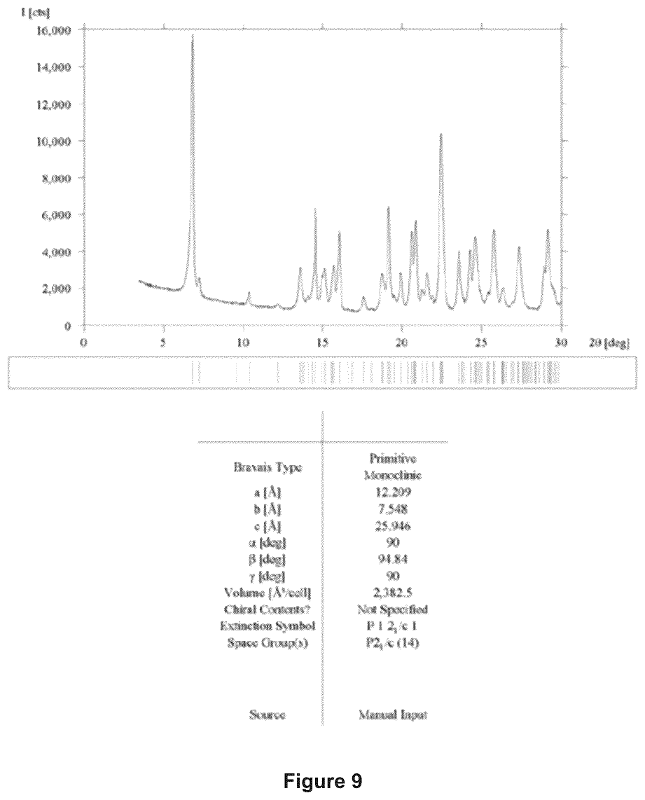

Aqueous slurry: the solid mixture was then slurried in water at RT (stirring for 1 day) and resulting solids isolated using a Swinnex filter assembly. The aqueous slurry afforded a single crystalline phase, Form A of Compound 3 was identified as the unsolvated mono-hydrochloride salt, as shown by the XRPD pattern on FIG. 8 (indexing of the pattern on FIG. 9).

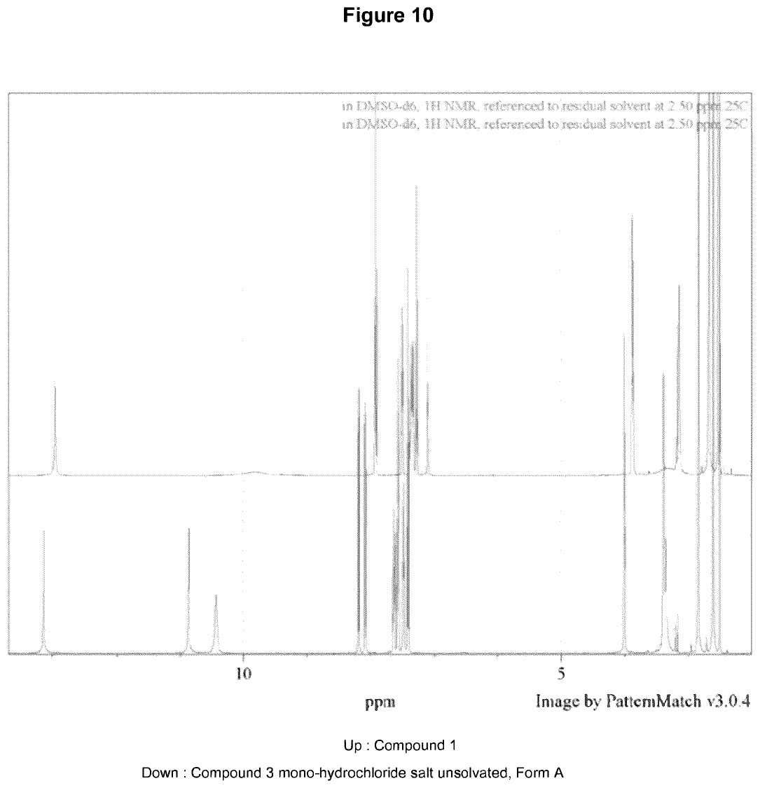

Proton NMR of Compound 3 is displayed on FIG. 10.
Solubility in water: <1 mg/mL.

Thermal analysis (TGA) conducted on the salt showed a weight loss of 1.0% from 54 to 120° C. (0.3 mol water). DSC analysis also revealed an endothermic peak (maxima) at 311° C. with an onset of 308° C. as displayed in FIG. 11.

Ion chromatography for the hydrochloride salt—Form A indicated 5.78% of chlorine (theoretical value: 6.65%—slight deviation could result from presence of residual solvent or impurities).

### Form B

Compound 3, Form B can be obtained from the following procedure: Charging compound 1 into H₂O (6 vol.). Adjusting pH to 3-4 with HCl (3 N) at 20-30° C. Stirring for 3.17 hours at 20-30° C. Adjusting the pH to 7-8 with 5% NaHCO₃ solution at 20-30° C. Stirring for 3.52 hours at 20-30° C. Filtering and washing the cake with H₂O (1 vol.). Charging the cake into 5% NaHCO₃ solution (6 vol.) and stirring for 4.4 hours at 20-30° C. Filtering and washing the cake with H₂O (1 vol.). Charging the cake into the co-solvent of MeOH/H₂O (1/4, 5 v.) for 7.3 hours at 20-30° C. Filtering and washing the cake with H₂O (1 vol.). Dried the cake at 55-65° C. to obtain Compound 3, Form B.

Characterization for Compound 3, Form B is given in FIGS. 25-26. Compound 3, Form B showed fine particle size with modest crystallinity. 6.4% of Cl— was detected by IC, indicated the stoichiometry of Form B was 1/1. There was no residual solvent detected by ¹H-NMR, while 1.6% of weight loss at 25-118° C. was observed by TGA due to loss of absorbed water. DSC exhibited two endothermic peaks at 50° C. and 301° C., corresponded to release of moisture and melting/decomposition, respectively.

Solubility of Compound 3 Form B was estimated in 30 solvents at room temperature and in 9 organic solvents at 70° C. using solvent addition method via visual assessment. The results are summarized in Table 2. Compound 3 Form B showed low solubility in most solvents at room temperature and modest solubility in DMSO, NMP, DMA and DMF at 70° C.

Protocol: about 3 mg of Compound 3 Form B was weighed into a sample vial and the solvent was added gradually with vortex until the drug solution was clear by observation or a total of 2 mL solvent was added. Then, the estimated solubility (mg/mL) was calculated.

TABLE 2
Solubility of Compound 3 Form B

| | Concentration | | | Concentration | |
|---|---|---|---|---|---|
| Solvent | (mg/mL) | Temp. | Solvent | (mg/mL) | Temp. |
| DMSO | 5.4 < S < 5.5 | RT | IPE | S < 1.5 | RT |
| NMP | 3.0 < S < 3.8 | | ACN | S < 1.5 | |
| DMA | 3.0 < S < 3.3 | | n-Heptane | S < 1.5 | |
| MeOH/Water | 2.5 < S < 3.0 | | DCM | S < 1.5 | |
| (9/1) | | | | | |
| DMF | 2.0 < S < 3.0 | | MIBK | S < 1.5 | |
| THF/Water | 1.6 < S < 1.9 | | Me- | S < 1.5 | |
| (9/1) | | | cyclohexane | | |
| ACN/Water | 1.5 < S < 1.7 | | DCM/MeOH | S < 1.5 | |
| (9/1) | | | (3/1) | | |
| Acetone/MeOH | 1.5 < S < 1.6 | | Acetone/Water | S < 1.5 | |
| (1/1) | | | (1/1) | | |
| MeOH | S < 1.5 | | Benzyl alcohol | S < 1.5 | |
| EtOH | S < 1.5 | | Acetic acid | S < 1.5 | |
| THF | S < 1.5 | | DMSO | S > 40 | 70° C. |
| 1,4-Dioxane | S < 1.5 | | NMP | S > 40 | |
| IPA | S < 1.5 | | DMA | 20 < S < 40 | |
| IPAC | S < 1.5 | | DMF | 16.7 < S < 20 | |
| MTBE | S < 1.5 | | Benzyl alcohol | S < 1.5 | |
| Water | S < 1.5 | | Acetone | S < 1.5 | |
| Acetone | S < 1.5 | | EtOH | S < 1.5 | |
| EA | S < 1.5 | | Acetic acid | S < 1.5 | |
| MEK | S < 1.5 | | THF | S < 1.5 | |
| 2-Me-THF | S < 1.5 | | | | |

### Form C

Compound 3, Form C can be obtained from MeOH by slurrying Compound 3, Form B at 50° C. for 4 days. Form C was recovered by filtration and dried under vacuum at 50° C. for 3 hours. The recovery yield is 74.9%.

Characterization for Compound 3, Form C is given in FIGS. 27-28. Fine particle size were obtained with modest crystallinity. 0.2% of residue of MeOH was detected by ¹H-NMR. TG analysis of Compound 3, Form C indicated no weight loss prior to melting. The DSC trace showed two endothermic peaks at 312° C. and 321° C. due to melting and decomposition, respectively. Therefore, Compound 3 Form C is an anhydrate.

### Form D

Compound 3, Form D can be obtained from DMSO/Water by reverse anti-solvent precipitation. 150 mg of Compound 3, Form B were dissolved in 2 mL of DMSO at 70° C. and then the solution was filtered at room temperature. The filtrate was charged in 20 mL of water (anti-solvent), and the suspension was stirred at room temperature for 4 days. Compound 3, Form D was collected by filtration and dried under vacuum at 50° C. for 3 hours. The recovery yield is 73.5%.

Characterization for Compound 3, Form D is given in FIGS. 29-30. Compound 3, Form D showed fine particle size with high crystallinity. There was 0.3% of residual DMSO detected by ¹H-NMR. The TGA curve showed 1.1% of weight loss at 25-90° C., ascribed to release of residual DMSO and moisture. The DSC thermogram showed four endothermic peaks. The small endothermic peak at 49° C. corresponded to exclusion of DMSO residue and moisture. The second endothermic peak at 280° C. was unknow yet. The sharp endothermic peak at 306° C. was due to melting. The decomposition took place at 316° C.

### Form E

Compound 3, Form E can be obtained from NMP/MTBE, DMA/MTBE, DMA/EA, DMA/IPAC or DMA/MIBK by anti-solvent precipitation, and from DMA/MTBE, DMA/EA or DMA/MIBK by reverse anti-solvent precipitation.

The sample obtained from DMA/MTBE by anti-solvent precipitation was further characterized. 150 mg of Compound 3, Form B was dissolved in 7 mL of DMA at 70° C. and then the solution was filtered at room temperature. 49 mL of MTBE (anti-solvent) was added in the filtrate in 10 hours. The suspension was stirred at room temperature for 4 days. Compound 3, Form E was collected by filtration and dried under vacuum at 50° C. for 3 hours. The recovery yield is 96.4%.

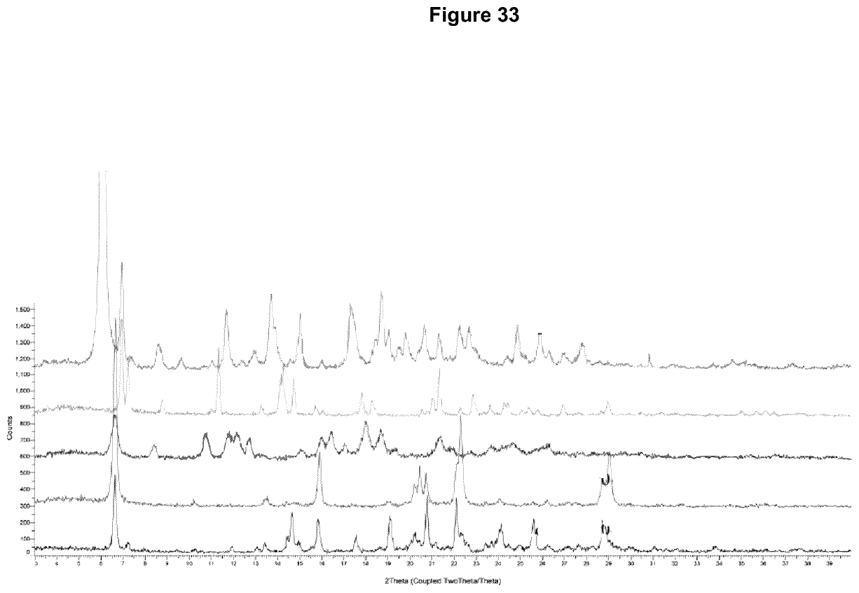

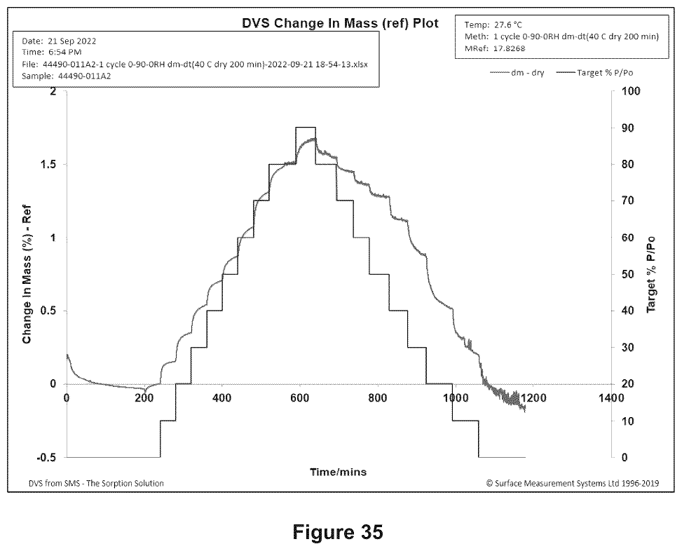

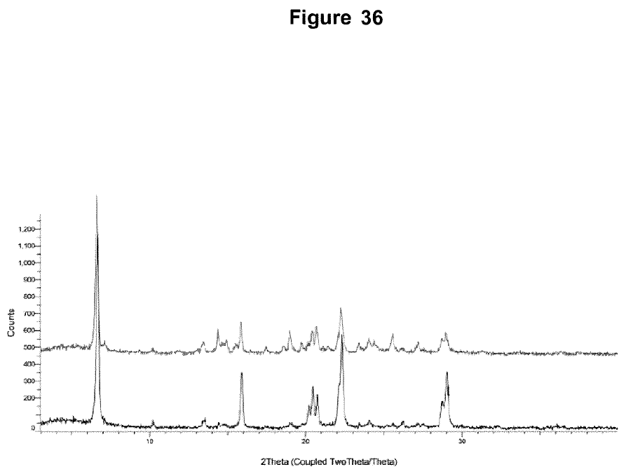

Characterization for Compound 3, Form E are given in FIGS. 31-36. Compound 3, Form E was characterized as irregular shaped crystals. There was around 17.6% of residual DMA and 0.4% of residual MTBE detected by ¹H-NMR. TGA thermogram of Compound 3, Form E showed 3.6% and 12.5% of weight losses at 25-110° C. and 110-160° C., attributed to release of residual solvents and desolvation, respectively. DSC showed two endothermic peaks at 73° C. and 130° C. (adjacent peak). Compound 3, Form E is a DMA (1 eq.) solvate.

### Comparison

A XRPD overlay shows the patterns for Compound 1, Free Forms D and E, and the patterns for Compound 3, Forms C, D and E.

A comparative study between Forms B, C, D and E revealed that Form D is the most stable form of Compound 3 in non-aqueous systems and in aqueous systems. Form D is slightly hygroscopic. It shows physical and chemical stability at 60° C. (capped) and 40° C. with 75% relative humidity (open).

### Example 6: Preparation of the Mesylate Salt

The mesylate salt was obtained via the following synthesis steps:

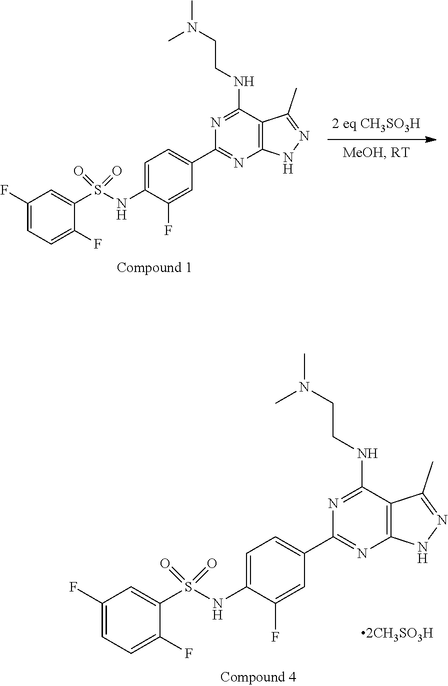 [SMILES: Cc1n[nH]c2nc(-c3ccc(NS(=O)(=O)c4cc(F)ccc4F)c(F)c3)nc(NCCN(C)C)c12 ; Cc1n[nH]c2nc(-c3ccc(NS(=O)(=O)c4cc(F)ccc4F)c(F)c3)nc(NCCN(C)C)c12 ; "·2CH3SO3H"]

1) Combine Compound 1 (99 mg) and 2 molar equivalents of methanesulfonic acid (26 μL) in methanol (1 mL) at room temperature.
2) The visually clear solution is allowed a slow evaporation (partial), stir at RT for 5 days.
3) Using a Swinnex filter assembly, isolate solids for XRPD analysis.

Compound 4 was obtained as a crystalline material. XRPD pattern analysis (FIGS. 12 and 13) and indexing of the pattern (FIG. 14) indicated the formation of an unsolvated di-mesylate salt of Compound 1.

### Example 7: Crystalline Forms of the Mesylate Salt

### Form A

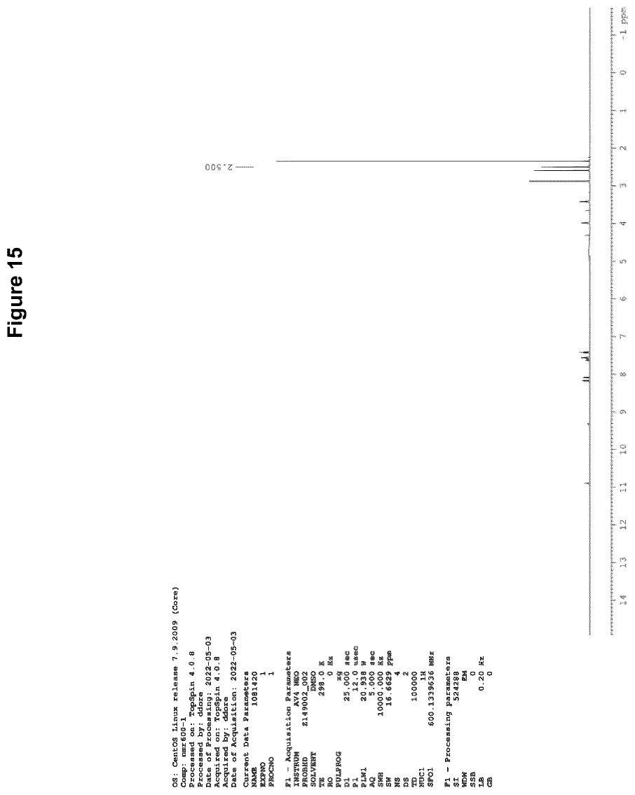

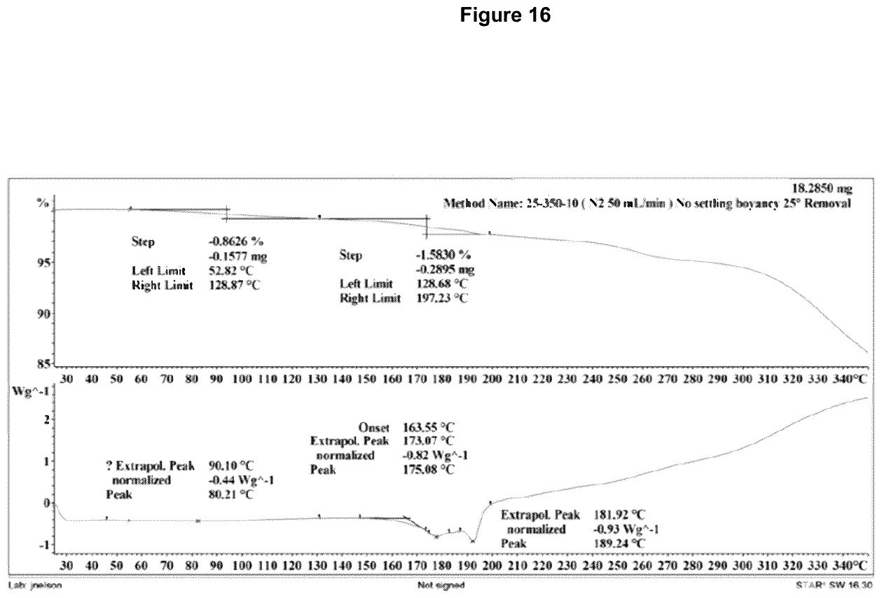

After being subjected to drying in a vacuum oven for 1 day at 62-72° C., Form A was characterized by XRPD (FIG. 22: pre-DVS) and proton NMR (FIG. 15). The results indicated that the Form A of the di-mesylate salt (unsolvated) is conserved.

Thermal analysis conducted on Form A showed a weight loss of 0.8% from 53 to 129° C. (0.3 mol water), and a weight loss of 1.6% from 129 to 197° C. DSC analysis also revealed a broad endothermic peak at 80° C. with an onset of 164° C. and two endothermic peaks temperature of 175° C. and 189° C. (peak maxima) as displayed in FIG. 16.

Aqueous solubility of Form A was determined by adding 0.1 mL of water to 2.5 mg of Mesylate Salt Form A. The initially clear solution was stirred at room temperature for 1 day and solids precipitated observed. Solubility in water is >25 mg/mL.

Solubility in MTBE is <2 mg/mL at RT.

### Form B

Compound 4 Form B can be obtained from various conditions: slurrying, slow cooling and anti-solvent precipitation. As summarized in Table 3.

TABLE 3
preparation conditions for Compound 4, Form B

| Solvent | Method | Procedure | Result |
|---|---|---|---|
| ACN | Slurry | Slurry at RT for 3 days | Form B |
| MeOH | Slurry | Slurry at RT for 3 days | Form B |
| ACN | Slurry | Slurry at 50° C. for 3 days | Form B |
| DMA/ACN | Anti-solvent | 7 mL of ACN added in 1 mL | Form B |
| | precipitation | of API/DMA solution | |
| | | (30 mg/mL) → stirred at | |
| | | RT for 1 day | |
| MeOH | Slow cooling | 50° C., 1 h → cooled to 2° | Form B |
| | | C. at 0.3° C./min → | |
| | | held at 2° C. for 17 h | |
| MeOH | Slow cooling | 50° C., 1 h → cooled to 2° | Form B |
| | | C. at 0.3° C./min → | |
| | | held at 2° C. for 17 h | |
| MeOH | Slow cooling | 60° C., 1 h → cooled to 40° | Form B |
| | | C. at 0.3° C./min → seeding → | (67% |
| | | cooled to 2° C. at 0.3° C./min | yield) |
| | | and held for 3 h → heated to 25° | |
| | | C. at 0.5° C./min and held for 2 | |
| | | h → cooled to 2° C. at 0.3° | |
| | | C./min and held for 3 h | |

The sample of Compound 4 Form B was then obtained by filtration and vacuum dried for 3 hours.

Compound 4 Form B showed needle-like shape with modest crystallinity. TGA indicated 5.4% of weight loss from 25 to 120° C. (FIG. 38). There was no residual solvent detected by ¹H-NMR, and the ratio of base/acid was detected as 1/2. The DSC trace showed one broad endothermic peak at 74° C. due to dehydration and one melting peak at 220° C. Therefore, di-mesylate Form 1 was likely a non-stoichiometric hydrate. The DVS isotherm of Compound 4 Form B was studied at 25° C., at 80% RH, the water uptake was 9.16%. The moisture uptake process can be reversed upon subsequently decreasing RH from 90% to 0%. The crystal form of Form 1 remained unchanged after DVS testing.

Solubility of Compound 4, Form B was estimated in 18 solvents at room temperature using solvent addition method via visual assessment. The results are summarized in Table 4. About 3 mg of Compound 4, Form B was weighed into a sample vial and the solvent was added gradually with vortex until the solution was clear by observation or a total of 2 mL solvent was added. Then, the estimated solubility (mg/mL) was calculated.

TABLE 4
Solubility assays for Compound 4,
Form B in various solvents (mg/mL)

| Solvent | Solubility (mg/mL) |
|---|---|
| DMSO | S > 150 |
| NMP | S > 150 |
| DMA | 75 < S < 150 |
| MeOH/Water (9/1, v/v) | 75 < S < 150 |
| Water | 75 < S < 150 |
| ACN/Water (9/1, v/v) | 15 < S < 20 |
| MeOH | 12 < S < 20 |
| EtOH | 3.75 < S < 7.5 |
| THF | S < 1.5 |
| DCM | S < 1.5 |
| IPA | S < 1.5 |
| IPAC | S < 1.5 |
| MTBE | S < 1.5 |
| ACN | S < 1.5 |
| Acetone | S < 1.5 |
| EA | S < 1.5 |
| MEK | S < 1.5 |
| 2-Me THF | S < 1.5 |

Compound 4, Form B showed low solubility in most solvents, and modest solubility in DMA, MeOH/Water (9/1, v/v), ACN/Water (9/1, v/v), MeOH, EtOH and water, while high solubility in DMSO and NMP.

### Form C

Compound 4, Form C is a metastable form obtained by slurrying of Compound 4, Form B in THF at RT and 50° C. for 3 days. In slurry experiment for re-preparing Compound 4 Form C starting from Compound 4 Form B, Form C occurred at 30 min and converted to di-mesylate Form D completely at 60 min. Therefore, only a mixture of Form C and trace Form B was obtained for characterization.

Compound 4 Form C was irregular shaped fine crystals with low crystallinity. There was around 5.0% (0.5 eq.) of residual THF detected by ¹H-NMR, and the ratio of base/acid was determined as %. TGA thermogram of Compound 4 Form C showed 0.8% and 3.5% weight loss at 30-95° C. and 95-165° C., might attribute to release of moisture and desolvation, respectively (FIG. 40). DSC trace showed three endothermic peaks at 61, 152 and 218° C., might correspond to exclusion of free moisture, desolvation and melting, respectively. Compound 4 Form C was a THF (0.5 eq.) solvate.

### Form D

Compound 4 Form D can be obtained from various conditions: slurrying, anti-solvent precipitation and reverse anti-solvent precipitation as summarized in Table 5.

TABLE 5
preparation conditions for Compound 4, Form D

| Solvent | Methods | Procedure | Result |
|---|---|---|---|
| DMSO/DCM | Anti-solvent | 6 mL of DCM was added | Form D |
| | precipitation | in 1.5 mL of API/DMSO | |
| | | solution (100 mg/mL). | |
| | | The suspension was stirred | |
| | | at RT for 3 days | |
| MTBE | Slurry | Stirred at 50° C. for 42 h | Form D |
| EtOH | Slurry | Stirred at 50° C. for 16 h | Form D |

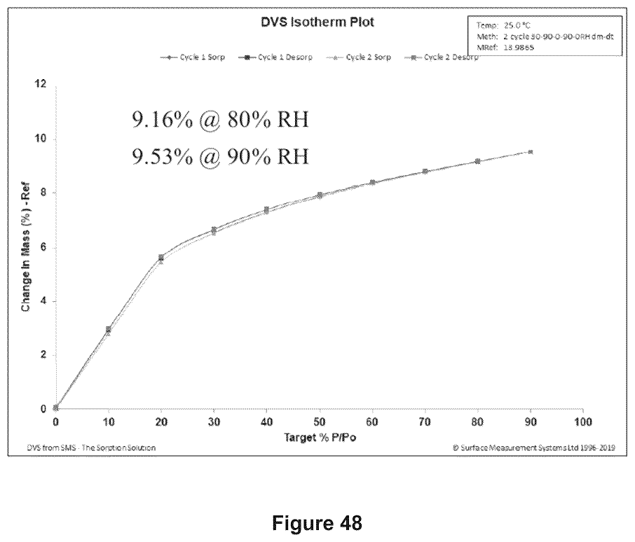

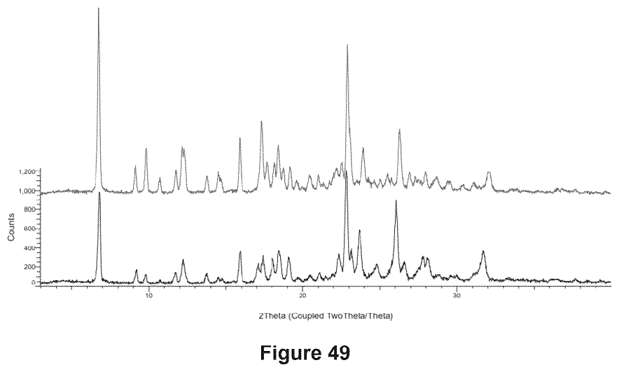

Form D was prepared at 150 mg scale. The resultant solids were collected by filtration and dried under vacuum at 50° C. for 3 h. The recovery yield was ˜81%. Form D showed irregular shape with modest crystallinity. There was 0.2% of MTBE residue detected by ¹H-NMR spectrum, and the ratio of base/acid was 1/2. The TGA result showed that Form D went through a procedure of dehydration from 90 to 157° C., corresponded to 2.4% of weight loss (FIG. 42). There were three endothermic and one exothermic peak in the DSC curve of Form D (FIG. 42). The first endothermic peak at 140° C. corresponded to dehydration. The second endothermic peak at 174° C. corresponded to melting of Form D. The followed exothermic peak ascribed to the form conversion from Form D to Form F. As the sample was heated up to elevated temperature, Form F melt at 219° C. (peak temperature). Form D was likely a monohydrate. DVS study of Form D indicated 0.99% of water uptake at 80% RH (FIG. 48). The moisture uptake process can be reversed with hysteresis upon subsequently decreasing RH from 90% to 0%. Moreover, the XRPD of Form D remained unchanged after DVS study (FIG. 49).

### Form E

Form E was obtained by slow cooling crystallization in NMP (FIG. 43). The unstable form has not been characterized further. A clear solution of Compound 4, Form B was dissolved (30 mg) in NMP at 50° C. and cooled from 50° C. to 2° C. Unstable Form E was obtained and converted to Form D after drying.

### Form F

Form F was obtained by heating Form D to 210° C. at a rate of 10° C./min and cooled to 25° C. at a rate of 5° C./min.

Form F showed irregular shapes with modest crystallinity. Humps were observed from XRPD pattern (FIG. 44), indicated amorphous phase was included in Form F. There was no solvents residue detected by ¹H-NMR spectrum, and the ratio of base/acid was %. The results of TGA showed 3.6% weight loss at 25-110° C. The DSC curve showed one broad endothermic peak at 61° C. due to dehydration and one melting peak at 217° C. (FIG. 45). The di-mesylate Form F was likely a non-stoichiometric hydrate with 1.5 eq. of water.

### Form G

Form G was obtained by slurrying Form B in water at 50° C. The preparation conditions are summarized in Table 6.

TABLE 6
Preparation conditions of mono-mesylate Form G

| Starting material | Procedure | Result |
|---|---|---|
| Form B | Slurry in water at RT for 3 d | Form G |
| Form B | Slurry in water at RT for 2 h | Form G |

Additionally, samples were vacuum dried at 50° C. for 3 hours to give Form G.

Samples of mono-mesylate Form G were agglomerates of irregular shapes with high crystallinity. There was no organic solvent residue detected by ¹H-NMR spectrum, and the ratio of base/acid was 1/1. Mono-mesylate Form G presented complicated thermal behaviors. The TGA analysis indicated 6.4% of weight loss at 25-100° C. The DSC curve showed four endothermic peaks at 96, 164, 239 and 266° C., and two exothermic peaks at 181 and 242° C. The mono-mesylate Form G was likely a hydrate (2.5 eq.) according to TGA and DSC thermograms.

### Comparison

Comparative study (interconversion and water activity) between Forms B to G revealed that Compound 4 Form D is the most stable form. Form D is stable at high water activity (0.16≤a_{W}≤0.90). Solid stability study indicated Form D was physically and chemically stable at 60° C. (close) and 40° C. at 75% RH for 7 days. Form D is slightly hygroscopic. Form D appears also to be more stable than Form A which is hygroscopic.

Form B also showed good stability properties despite its hygroscopic nature.

### Example 8: Dynamic Vapor Sorption/Desorption (DVS) Experiments

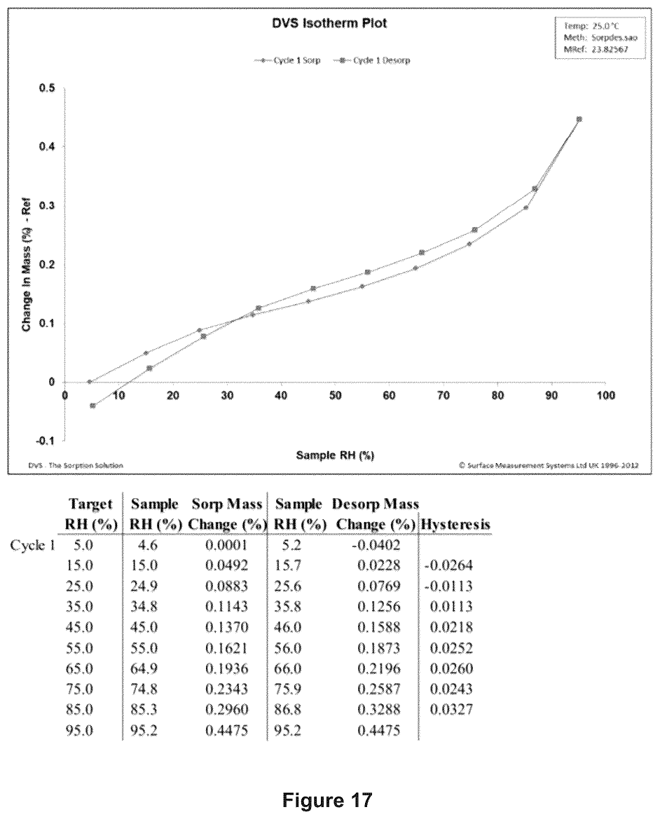

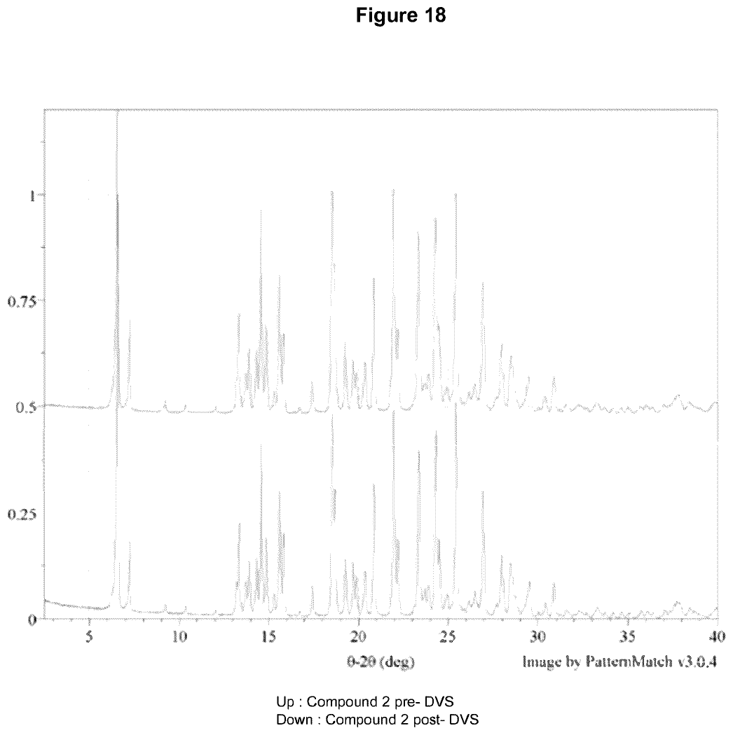

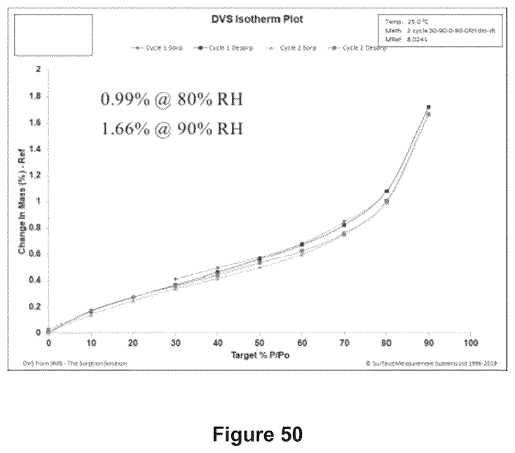

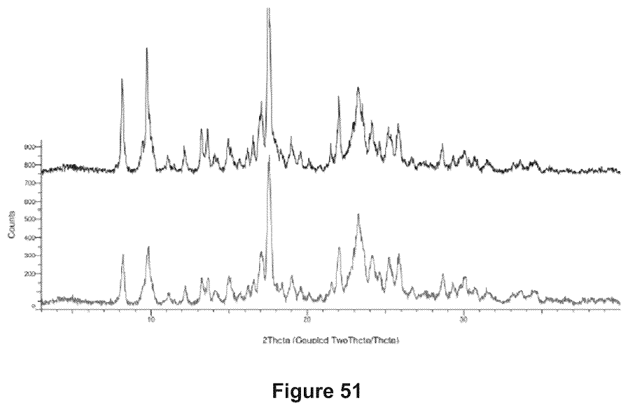

DVS experiments (FIGS. 17, 19, 21, 34, 35, 48 and 50) and XRPD post-DVS experiments (FIGS. 18, 20, 22, 36, 49 and 51) were conducted in order to study the deliquescence behaviour of the crystalline salts upon expose to humidity.

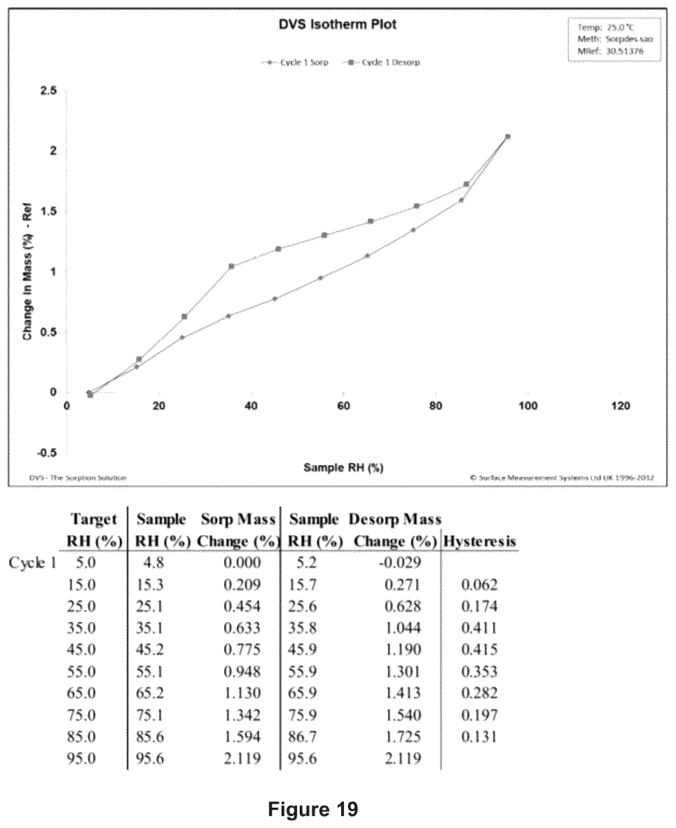

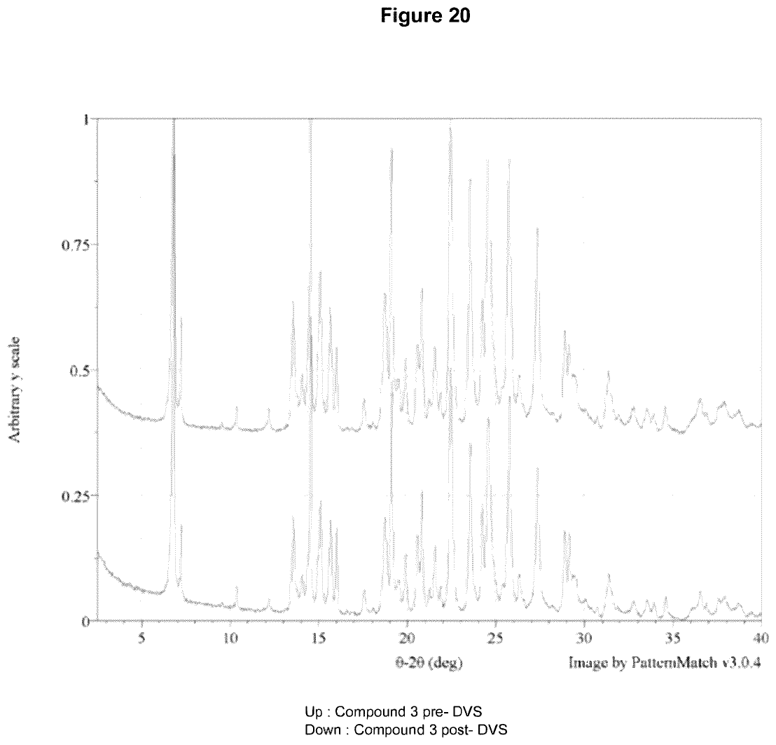

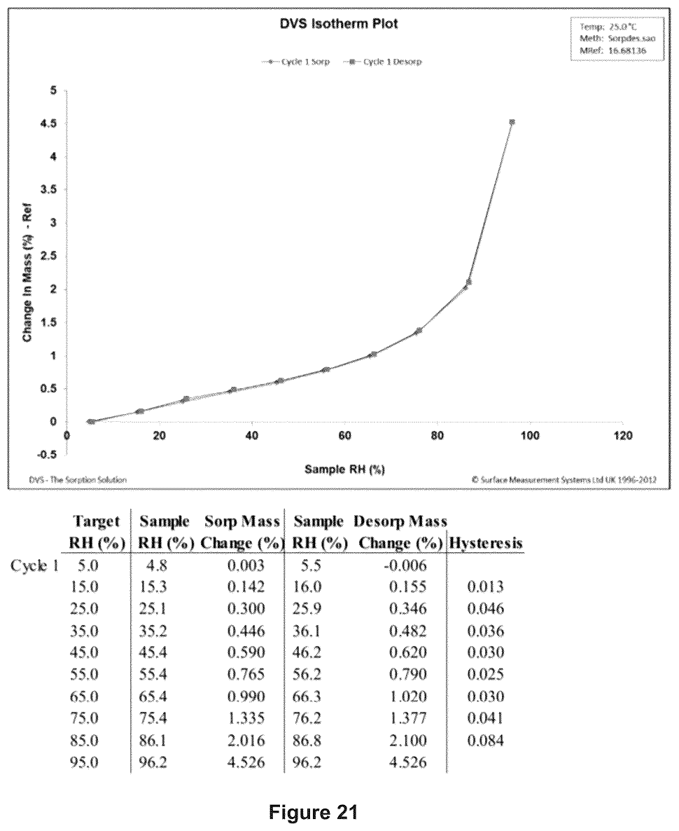

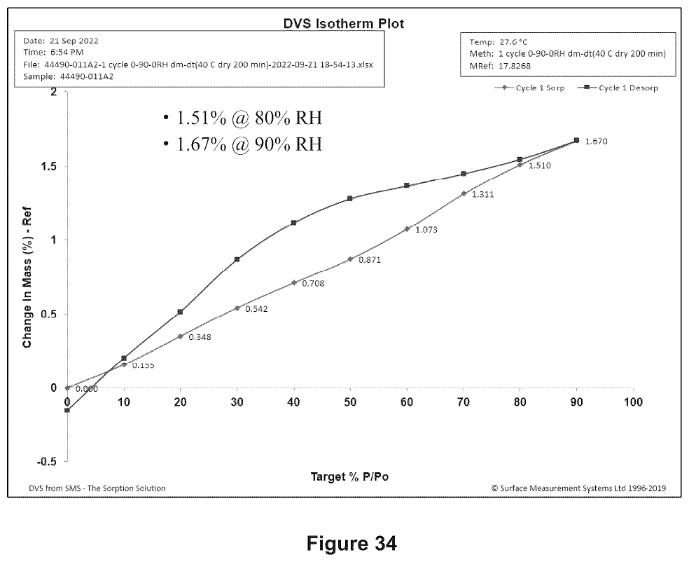

TABLE 7
Summary of the DVS experiments and corresponding XRPDs conducted for the formate
salt (Compound 2, Form A), the hydrochloride salt (Compound 3, Form A and Form
D) and the di-mesylate salt (Compound 4, Form A, Form B and Form D).

| | | | Post-DVS | |
|---|---|---|---|---|
| | wt % | Reversibility | XRPD | Comments |
| Formate salt | 0.4 wt % | Reversible with minor | No change | Non- |
| Form A | gain from 5 | hysteresis | | hygroscopic |
| Compound 2 | to 95% RH | | | |
| FIGS. 17-18 | | | | |
| Hydrochloride salt | 2.1 wt % | Reversible with some | No change | Slightly |
| Form A | gain from 5 | hysteresis | | hygroscopic |
| Compound 3 | to 96% RH | | | |
| FIGS. 19-20 | | | | |
| Hydrochloride salt | 1.5 wt % | Minor hysteresis | Extra peak at | Slightly |
| Form D | and 1.7 | | 7° (2θ) | hygroscopic |
| Compound 3 | wt % gain | | (attributed to | Form D prone |
| FIGS. 34-36 | from 80 to | | freebase) | to dissociation |
| | 90% RH | | | at high RH |
| Di-mesylate salt | 1.3 wt % | Reversible | No change | Hygroscopic |
| Form A | gain from 5 | | | |
| Compound 4 | to 75% RH | | | |
| FIGS. 21-22 | 3.2 wt % | | | |
| | gain from | | | |
| | 75 to 96% | | | |
| | RH | | | |
| | TOTAL | | | |
| | WEIGHT | | | |
| | GAIN: | | | |
| | 4.5% or 1.8 | | | |
| | mol water | | | |
| Di-mesylate salt | 9.16 wt % | Reversible | No change | Hygroscopic |
| Form B | gain at 25° | | | |
| Compound 4 | C. and | | | |
| FIGS. 37-38 | 80% RH | | | |
| Di-mesylate salt | 0.99 wt % | Reversible with some | No change | Slightly |
| Form D | gain at | hysteresis upon | | hygroscopic |
| Compound 4 | 80% RH | subsequently decreasing | | |
| FIGS. 41-42 | | RH from 90% to 0% | | |

### Example 9: Water Activity Studies

### Competition Study for Compound 3 Form B and Form D at Different Water Activities

Water activity study was performed using Compound 3 Form B (anhydrate) as the input material in EtOH/Water with different water content at RT. About 5 mg of Compound 3, Form B was used to prepare saturated solutions and the solutions were filtered by syringe at RT. 5 mg of Compound 3, Form D were added in the 1 mL of filtrate, and the suspension was slurried at RT for 3 and 7 days. The solids collected after filtration were analyzed by XRPD. Data are summarized in Table 8.

TABLE 8
Water activity study results

| | | | | | XPRD results | | |
|---|---|---|---|---|---|---|---|
| Input | | EtOH | Water | | 3 | 7 | 7 |
| Compound 3 | T | (V/%) | (V/%) | a_{W} | D/Wet | D/Wet | D/Dried |

| | | | | | |
|---|---|---|---|---|---|
| Form B + D | RT | 98.4 | 1.6 | 0.12 | Form D |
| Form B + D | RT | 97 | 3 | 0.21 | Form D |
| Form B + D | RT | 92.8 | 7.2 | 0.41 | Form D |
| Form B + D | RT | 82.9 | 17.1 | 0.64 | Form D |
| Form B + D | RT | 58.1 | 41.9 | 0.82 | Form D + extra peak |
| Form B + D | RT | 24.7 | 75.3 | 0.92 | Form D + extra peak |

It was found that Compound 3, Form D is stable at water activity lower than 0.6. An extra peak at 7° was observed at water activities of 0.8 and 0.9, indicating that Compound 3, Form D might be converted to the freebase at high water activity.

### Study for Compound 4 Form B and Form D

Water activity study between Compound 4 Form B and D was conducted in MeOH/water, ACN/water and IPAC/water at room temperature. The experimental results are shown in Table 9. It could be concluded that Compound 4 Form D was the most stable form at high water activity, 0.16≤a_{w}≤0.90, while Compound 4 Form B was the most stable form at low water activity, a_{w}≤0.08.

Protocol: excess amount of Compound 4 Form B was used to prepare saturated solutions. 6-8 mg each of Compound 4 Form B and Form D were added in the solutions. The suspension was slurried at room temperature for the corresponding times. Residual solids were collected by filtration with syringe filter, and characterized by XRPD.

TABLE 9
Water activity study results

| Input | Solvent | | XPRD results | | | |
|---|---|---|---|---|---|---|
| Compound 4 | (V/V) | a_{W} | Time | Form | Time | Form |
| Forms B + D | ACN/water | 0.37 | 8 d | Form D | 14 d | Form D |
| Forms B + D | ACN/water | 0.58 | 8 d | Form D | 14 d | Form D |
| Forms B + D | ACN/water | 0.78 | 8 d | Form D | 14 d | Form D |
| Forms B + D | ACN/water | 0.90 | 8 d | Form D | 14 d | Form D |
| Forms B + D | MeOH/water | 0.08 | 3 d | Forms B + D | 7 d | Form B |
| Forms B + D | MeOH/water | 0.16 | 3 d | Form D | 7 d | Form D |

a_{w} was calculated using Dynochem with NRTL model.

### Example 10: Solid Stability Experiments

### Study for Compound 3 Form D

Solid stability testing of Compound 3, Form D was evaluated at the conditions of 60° C. (capped) and 40° C./75% RH (open) for 7 days. Polymorphic form and purity were analyzed by XRPD and HPLC, respectively. The experimental results are given in Table 10. It was found that Compound 3, Form D is chemically and physically stable at 60° C. (capped) and 40° C./75% RH (open) for 7 days.

TABLE 10
Solid stability results evaluated by HPLC and XRPD

| | Purity - 7 days | | | XRPD - 7 days | | |
|---|---|---|---|---|---|---|
| | Initial | 40° C./ | | Initial | 40° C./ | |
| Sample | T0 | 75% RH | 60° C. | T0 | 75% RH | 60° C. |
| Com- | 99.19% | 99.30% | 99.24% | Form D | Form D | Form D |
| pound 3, | | | | | | |
| Form D | | | | | | |

### Study for Compound 4 Form B and Form D

Solid stability testing of Compound 4 Form B and Form D were evaluated at the conditions of 60° C. (vial close) and 40° C./75% RH (vial open) for 7 days. Polymorphic form and purity were checked by XRPD and HPLC, respectively. The experimental results are given in Table 11.

Form D was chemically and physically stable at 60° C. (close) and 40° C./75% RH (open) for 7 days, while Form B was chemically and physically stable at 60° C. (close), but partially converted to Form D at 40° C./75% RH (open) for day 7.

TABLE 11
Solid stability results evaluated by HPLC and XRPD

| | Purity - 7 days | | | XRPD - 7 days | | |
|---|---|---|---|---|---|---|
| | Initial | 40° C./ | | Initial | 40° C./ | |
| Sample | T0 | 75% RH | 60° C. | T0 | 75% RH | 60° C. |
| Com- | 99.47% | 99.18% | 99.41% | Form B | Form B + | Form B |
| pound 4 | | | | | Form D | |
| Form B | | | | | | |
| Com- | 99.29% | 99.29% | 99.17% | Form D | Form D | Form D |
| pound 4 | | | | | | |
| Form D | | | | | | |

### Example 11: Slurry Experiments

### Slurry Study for Compound 3 Form B

About 20 mg of Compound 3, Form B was weighed into a sample vial and then 1 mL of solvent was added to make a suspension. The suspension was stirred at room temperature or 50° C. for 3 days and 11 days. Then, the wet solid was collected by filtration and analyzed by XRPD.

TABLE 12
Slurry study on Compound 3, Form B evaluated by XRPD

| | At RT for 3 days | | At 50° C. for 3 days | |
|---|---|---|---|---|
| | and 11 days | | and 11 days | |
| | XRPD | | XRPD | |
| | Day 3/ | Day 11/ | Day 3/ | Day 11/ |
| Solvent | Wet | Wet | Wet | Wet |
| MeOH, 1 mL | Amorphous | Form D | Form D | Form D |
| EtOH, 1 mL | Form D | | | |
| THF, 1 mL | Amorphous | | | |
| 1,4-Dioxane, 1 mL | Form D | | | |
| IPA, 1 mL | | | | |
| IPAC, 1 mL | | | | |
| MTBE, 1 mL | | | | |
| Water, 1 mL | | | | |
| n-Heptane, 1 mL | | | | |
| Acetone, 1 mL | | | | |
| EA, 1 mL | | | | |
| ACN, 1 mL | | | | |
| DCM, 1 mL | | | | |
| MIBK, 1 mL | | | | |
| MEK, 1 mL | | | | |

Competitive slurry experiment for Compound 4 Form B and Form D Competitive slurry experiments of Compound 4 Form B and Form D were set up to verify the relative stability relationship. The experimental results are shown in Table 13. It was supposed that Form D was the stable form at high water activity, while Form B was stable at low water activity. All the solvents used was analytical grade and should contained trace amounts of water. IPAC showed high water activity with the presence of small amount of water. Hence, Form B was converted to Form D in IPAC at both room temperature and 50° C. MeOH exhibited lowest water activity with trace water, and thus Form B was isolated as the products. The water activity at elevated temperature is lower than at low temperature. Therefore, Form B was obtained in ACN at 50° C., while Form D was the stable form at room temperature.

TABLE 13
Competitive slurry study on Compound 4,
Form B and Form D evaluated by XRPD

| | | | Results | |
|---|---|---|---|---|
| Input | Solvent | Temp. | 8 d | 14 d |
| Form B + | MeOH | RT | Form B | Form B |
| Form D | ACN | | Form B + Form D | Form D |
| | IPAC | | Form D | Form D |
| | MeOH | 50° C. | Form B | Form B |
| | ACN | | Form B | Form B |
| | IPAC | | Form D | Form D |

### Slurry Study for Compound 4

20-30 mg of Compound 4 Form B was added in 1 mL of selected solvents at room temperature or 50° C. The suspension was slurried at room temperature or 50° C. for 3 days and 11 days. The wet solids collected from filtration were examined by XRPD. All results for slurry experiments are summarized in Table 14. There were four crystal forms obtained by slurrying, including di-mesylate Forms B, C and D as well as mono-mesylate Form G.

TABLE 14
Slurry study on Compound 4 evaluated by XRPD

| | At RT for 3 days and 11 days | | | At 50° C. for 3 days and 11 days | | |
|---|---|---|---|---|---|---|
| | | XRPD Result | | | XRPD Result | |
| Solvent | Input | Day 3/Wet | Day 11/Wet | Input | Day 3/Wet | Day 11/Wet |
| ACN | 20 mg | Form B | Form B | 20 mg | Form B | Form B |
| DCM | | Form D | Form D | | Form B | N/A¹ |
| THF | | Form C | Form D | | Form C | Form B + Form |
| | | | | | | D + extra peak |
| IPA | | Form D | Form D | | Form D | Form D |
| IPAC | | Form D | Form D | | Form D | Form D |
| MTBE | | Form B + | Form D | | Form D | N/A¹ |
| | | Form D | | | | |
| Acetone | | Form B + | Form D | | Form C | Form D |
| | | Form D | | | | |
| EA | | Form B + | Form D | | Form D | Form D |
| | | Form D | | | | |
| MEK | | Form D | Form D | | Form D | Form D |
| 2-Me THF | | Form D | Form D | | Form D | Form D |
| ACN/Water | 30 mg | Form D | Form D | N/A | N/A | N/A |
| (9/1, v/v) | | | | | | |
| MeOH | | Form B | Form B | N/A | N/A | N/A |
| EtOH | | Form D | Form D | N/A | N/A | N/A |

| | |
|---|---|
| Water | The suspensions were slurried at RT or 50° C. for 3 days. |
| | Mono-mesylate Form G was obtained. |

N/A: non applicable
¹The boiling points of DCM and MTBE are 40° C. and 55° C., respectively. The solvents were evaporated to dryness at 50° C.

### Example 12: Cooling/Anti-Solvent Precipitation/Reverse Anti-Solvent Precipitation Experiments

### Cooling

### Cooling Experiments for Compound 3 Form B

Clear solutions were prepared by dissolving 20 mg of Compound 3 in corresponding solvents at 70° C. The solution was cooled from 70° C. to 2° C. The procedure and results are shown in Table 15. There was no precipitate obtained from the cooling experiments.

TABLE 15
Procedure and results of cooling crystallization
of Compound 3 Form B

| Solvent, | | XRPD resulting |
|---|---|---|
| volume | Cooling Process | Form |
| DMF, 60 V | 70° C., 1 hour → cooled to | Clear solution |
| DMA, 50 V | 2° C. at 0.3° C./min → | |
| DMSO, 25 V | held at 2° C. overnight | |
| NMP, 25 V | | |

### Cooling Experiments for Compound 4 Form B

Clear solutions were prepared by dissolving 30 mg of Compound 4 Form B in corresponding solvents at 50° C. The solutions were cooled from 50° C. to 2° C. The procedure and results are shown in Table 16.

TABLE 16
Procedure and results of cooling crystallization
of Compound 4 Form B

| Solvent, | | XRPD resulting |
|---|---|---|
| volume | Cooling Process | Form |
| MeOH, 50 V | 50° C., 1 hour → cooled to | Form B |
| DMA, 10 V | 2° C. at 0.3° C./min → | Clear solution |
| DMSO, 10 V | held at 2° C. for 17 hours | Clear solution |
| NMP, 10 V | | Wet: Form E |
| | | Dry: Form D + |
| | | traces of Form E |

### Anti-Solvent Precipitation

### Anti-Solvent Precipitation Experiments for Compound 3 Form B

Compound 3 Form B was dissolved in 1 mL of DMSO, NMP or DMA at 70° C. with the concentration of 30 mg/mL or 15 mg/mL. The filtrate was charged in 8 mL or 20 mL vials at room temperature. Anti-solvent (S<1.5 mg/mL) was charged in until precipitation occurred or the volume ratio of solvent to anti-solvent reached 1 to 15. Solid was isolated by filtration and analyzed by XRPD. All results are given in Table 17. Compound 3 Form D was obtained from most of solvents. Compound 3 Form E was obtained from NMP/MTBE, DMA/MTBE, DMA/EA, DMA/IPAC and DMA/MIBK. Compound 1 Freebase Form D was obtained from NMP/Water, and a mixture of freebase Forms D and E was obtained from DMA/Water.

TABLE 17
Procedure and results of anti-solvent
precipitation of Compound 3 Form B

| | | Stirring | | |
|---|---|---|---|---|
| Solvent | Anti-solvent | time | | XPRD resulting Form |
| DMSO, | EtOH, 15 mL | 4 | Days | Clear solution |
| 1 mL | Acetone, 15 mL | 4 | Days | Clear solution |
| 30 mg/mL | 2-Me—THF, | 4 | Days | Clear solution |
| | 15 mL | | | |
| | MTBE, 15 mL | 1 | Day | Oil |
| | EA, 15 mL | 4 | Days | Wet: Form D |
| | ACN, 15 mL | 4 | Days | Wet: Form D |
| | DCM, 15 mL | 4 | Days | Wet: Form D |
| | Water, 15 mL | 1 | Day | Wet: Form D + extra peak |
| | | | | Dry: Form D |
| NMP, | EtOH, 15 mL | 4 | Days | Clear solution |
| 1 mL | Acetone, 15 mL | 4 | Days | Clear solution |
| 30 mg/mL | 2-Me—THF, | 4 | Days | Clear solution |
| | 15 mL | | | |
| | MTBE, 7 mL | 1 | Day | Wet: Form E |
| | | | | Dry: Form E |
| | EA, 10 mL | 2 | Days | Wet: Form D |
| | ACN, 7 mL | 1 | Day | Wet: Form D |
| | DCM, 7 mL | 1 | Day | Wet: Form D |
| | Water, 10 mL | 4 | Days | Wet: Form D |
| | | | | Dry: Form D |
| DMA, | EtOH, 15 mL | 4 | Days | Clear solution |
| 1 mL | Acetone, 15 mL | 4 | Days | Clear solution |
| 15 mg/mL | 2-Me—THF, | 4 | Days | Clear solution |
| | 15 mL | | | |
| | MTBE, 7 mL | 1 | Day | Wet: Form E |
| | EA, 8 mL | 2 | Days | Wet: Form E |
| | ACN, 7 mL | 1 | Day | Wet: Form D |
| | DCM, 10 mL | 2 | Days | Wet: Form D |
| | Water, 10 mL | 4 | Days | Wet: Mixed forms of freebase |
| | | | | (Compound 1) |
| | | | | Dry: Mixed forms of freebase |
| | | | | (Compound 1) |
| | IPA, 15 mL | 4 | Days | Clear solution |
| | MEK, 15 mL | 4 | Days | Clear solution |
| | IPAC, 7 mL | 1 | Day | Wet: Form E |
| | MIBK, 7 mL | 2 | Days | Wet: Form E |
| | MeOH, 15 mL | 4 | Days | Clear solution |

### Anti-Solvent Precipitation Experiments for Compound 4 Form B

30 mg of Compound 4 Form B were dissolved in 1 mL of DMSO or DMA at room temperature (concentration of 30 mg/mL). The filtrate was charged in 8 mL vials at room temperature. Anti-solvent (S<1.5 mg/mL) was charged in until precipitation occurred or the volume ratio of solvent to anti-solvent reached 1 to 7. Solid was isolated by filtration and analyzed by XRPD. All results are given in Table 18. Compound 4 Form B was obtained from DMA/ACN. Compound 4 Form D was obtained from DMA/DCM and DMSO/DCM. The mixture of Compound 4 Forms B and D was obtained from DMSO/ACN and DMA/Acetone, and a mixture of Compound 4 Form D and trace Forms B or C was obtained from several solvent systems.

TABLE 18
Procedure and results of anti-solvent
precipitation of Compound 4 Form B

| | | Stirring | | |
|---|---|---|---|---|
| Solvent | Anti-solvent | time | | XPRD resulting Form |
| DMSO, | Acetone, 7 mL | 2 | Days | Trace solids |
| 1 mL | 2-Me THF, 7 mL | 1 | Day | Form D + trace of Form C |
| | EA, 7 mL | 1 | Day | Form D + trace of Form C |
| | ACN, 7 mL | 6 | Days | Form D + trace of Form B |
| | DCM, 7 mL | 1 | Day | Form D |
| DMA, | Acetone, 7 mL | 6 | Days | Form B + trace of Form D |
| 1 mL | 2-Me THF, 7 mL | 1 | Day | Trace solids |
| | MTBE, 7 mL | 1 | Day | Trace solids |
| | EA, 7 mL | 1 | Day | Trace solids |
| | ACN, 7 mL | 1 | Day | Form B |
| | DCM, 7 mL | 1 | Day | Form D |

### Reverse Anti-Solvent Precipitation

### Reverse Anti-Solvent Precipitation Experiments for Compound 3 Form B

The Compound 3 Form B was dissolved in selected solvent at 50° C. with the concentration of 30 mg/mL or 15 mg/mL. Then 1 mL of solution was filtered at room temperature and added in anti-solvent (solvent/anti-solvent, v/v, 1/10). The precipitates were collected and analysed by XRPD. All results are given in Table 19. Compound 1 freebase Form D was obtained from DMSO/Water, and Compound 3 Form E was obtained from DMA/MTBE, DMA/EA, DMA/MIBK. In addition, mixed forms of Compound 1 freebase was obtained from DMA/Water and NMP/Water.

TABLE 19
Procedure and results of reverse anti-solvent
precipitation of Compound 3 Form B

| | | Stirring | | |
|---|---|---|---|---|
| Solvent | Anti-solvent | time | | XPRD resulting Form |
| DMSO, | Water, 10 mL | 1.5 | Hours | Wet: Compound 1 freebase |
| 1 mL | | | | Form D |
| 30 mg/mL | | | | |
| DMA, | MTBE, 10 mL | 1.5 | Hours | Wet: Compound 3 Form E |
| 1 mL | EA, 10 mL | 2 | Days | Wet: Compound 3 Form E |
| 15 mg/mL | ACN, 10 mL | 1 | Day | Wet: Compound 3 Form D |
| | DCM, 10 mL | 1 | Day | Wet: Compound 3 Form D |
| | Water, 10 mL | 2 | Days | Mixed forms of Compound |
| | | | | 1 freebase |
| | IPAC, 10 mL | 1.5 | Hours | Compound 3 Form D |
| | MIBK, 10 mL | 2 | Days | Compound 3 Form E |
| NMP, | MTBE, 10 mL | 4 | Days | Compound 3 Form D |
| 1 mL | EA, 10 mL | 4 | Days | Compound 3 Form D |
| 30 mg/mL | ACN, 10 mL | 4 | Days | Compound 3 Form D |
| | DCM, 10 mL | 1 | Day | Compound 3 Form D |
| | Water, 10 mL | 2 | Days | Wet: Mixed forms of |
| | | | | Compound 1 freebase + |
| | | | | extra peak |
| | | | | Dry: Mixed forms of |
| | | | | Compound 1 freebase |

### Reverse Anti-Solvent Precipitation Experiments for Compound 4 Form B

Compound 4 Form B was dissolved in selected solvent at room temperature with the concentration of 30 mg/mL. Then 1 mL of solution was filtered at room temperature and added in the anti-solvent (solvent/anti-solvent, v/v, 1/10). The precipitates were collected and analyzed by XRPD. All results are given in Table 20. The mixture of Compound 4 Form B and Form D was obtained from most solvents, and Compound 4 Form D was obtained from DMSO/2-Me THF, DMSO/EA, DMSO/DCM, DMA/2-Me THF and DMA/EA.

TABLE 20
Procedure and results of reverse anti-solvent
precipitation of Compound 4 Form B

| | | Stirring | | |
|---|---|---|---|---|
| Solvent | Anti-solvent | time | | XPRD resulting Form |
| DMSO, | Acetone, 10 mL | 1 | Day | Trace solids |
| 1 mL | 2-Me THF, 10 mL | 1 | Day | Form D |
| | MTBE, 10 mL | 1 | Day | Trace solids |
| | EA, 10 mL | 1 | Day | Form D |
| | ACN, 10 mL | 6 | Days | Form B + trace of Form D |
| | DCM, 10 mL | 1 | Day | Form D |
| DMA, | Acetone, 10 mL | 6 | Days | Form B + trace of Form D |
| 1 mL | 2-Me THF, 10 mL | 1 | Day | Form D |
| | MTBE, 10 mL | 2 | Hours | Form B + trace of Form D |
| | EA, 10 mL | 1 | Day | Form D |
| | ACN, 10 mL | 1 | Day | Trace solids |
| | DCM, 10 mL | 2 | Hours | Form B |
| | | 1 | Day | Form B + trace of Form D |

### Example 13: Thermal Treatment

### Thermal Study for Compound 3 Form E

Compound 3, Form E was treated with heat-cool cycle by DSC. The resulted solids were characterized by XRPD. Compound 3, Form E, was converted to Compound 3, Form D after desolvation.

TABLE 21
Thermal study on Compound 3, Form E evaluated by XRPD

| Initial | | XRPD |
|---|---|---|
| Form | Thermal Treatment | Resulting Form |
| Form E | Heated to 114° C. at 10° C./min, and | Form E |
| | cooled to 25° C. at 5° C./min. | |
| Form E | Heated to 160° C. at 10° C./min, and | Form D |
| | cooled to 25° C. at 5° C./min. | |

### Thermal Study for Compound 4 Form B

Compound 4 Form B was treated with heat-cool cycle by DSC. The resulted solids were checked by XRPD. All results are given in Table 22. The XRPD pattern kept unchanged after dehydration, that suggested it was a non-stoichiometric hydrate. The water was excluded at elevated temperature, then entered in the crystal lattice again at room temperature.

TABLE 22
Thermal study on Compound 4, Form B evaluated by XRPD

| Initial | | XRPD |
|---|---|---|
| Form | Thermal Treatment | Resulting Form |
| Form B | Heated to 130° C. at 10° C./min, and | Form B |
| | cooled to 25° C. at 5° C./min. | |

### Thermal Study for Compound 4 Form C and Trace of Form B

Compound 4 Form C and trace Form B was treated with heat-cool cycle by DSC. The resulted solids were checked by XRPD. All results are given in Table 23. The sample was converted to low crystalline (LC) Form B after heating to 175° C.

TABLE 23
Thermal study on Compound 4, Form D evaluated by XRPD

| Initial | | XRPD |
|---|---|---|
| Form | Thermal Treatment | Resulting Form |
| Form C + | Heated to 175° C. at 10° C./min, and | Form B (LC) |
| trace Form B | cooled to 25° C. at 5° C./min. | |

### Thermal Study for Compound 4 Form D

Compound 4 Form D was treated with heat-cool cycle by DSC. The resulted solids were checked by XRPD. All results are given in Table 24. The sample kept unchanged after heating to 140° C., but was converted to a new low crystalline form after heating to 210° C. which was assigned to Form F.

TABLE 24
Thermal study on Compound 4, Form D evaluated by XRPD

| Initial | | XRPD |
|---|---|---|
| Form | Thermal Treatment | Resulting Form |
| Form D | Heated to 140° C. at 10° C./min, and | Form D |
| | cooled to 25° C. at 5° C./min. | |
| Form D | Heated to 210° C. at 10° C./min, and | Form F |
| | cooled to 25° C. at 5° C./min. | |

### XPRD Peak Lists

### Observed Peaks for Compound 1, Form D:

| | | | Net In- | Rel. In- | | Width |
|---|---|---|---|---|---|---|
| Name | Angle/° | d Value/Å | tensity | tensity | FWHM | (low) |
| Peak #1 | 6.948 | 12.71268 | 356.032 | 100.0% | 0.152 | 0.076 |
| Peak #2 | 7.245 | 12.19204 | 225.041 | 63.2% | 0.170 | 0.085 |
| Peak #3 | 8.795 | 10.04582 | 56.7592 | 15.9% | 0.100 | 0.050 |
| Peak #4 | 10.997 | 8.03887 | 27.629 | 7.8% | 0.100 | 0.050 |
| Peak #5 | 11.332 | 7.80201 | 287.946 | 80.9% | 0.100 | 0.050 |
| Peak #6 | 13.259 | 6.67200 | 39.1321 | 11.0% | 0.100 | 0.050 |
| Peak #7 | 14.259 | 6.20664 | 174.369 | 49.0% | 0.136 | 0.068 |
| Peak #8 | 14.737 | 6.00630 | 150.953 | 42.4% | 0.100 | 0.050 |
| Peak #9 | 15.742 | 5.62501 | 33.9253 | 9.5% | 0.138 | 0.069 |
| Peak #10 | 17.846 | 4.96621 | 104.513 | 29.4% | 0.100 | 0.050 |
| Peak #11 | 18.307 | 4.84220 | 46.8167 | 13.1% | 0.102 | 0.051 |
| Peak #12 | 20.563 | 4.31568 | 35.2758 | 9.9% | 0.155 | 0.077 |
| Peak #13 | 21.047 | 4.21766 | 66.0118 | 18.5% | 0.100 | 0.050 |
| Peak #14 | 21.349 | 4.15864 | 212.36 | 59.6% | 0.100 | 0.050 |
| Peak #15 | 22.296 | 3.98412 | 34.9949 | 9.8% | 0.100 | 0.050 |
| Peak #16 | 22.863 | 3.88660 | 92.2583 | 25.9% | 0.100 | 0.050 |
| Peak #17 | 23.645 | 3.75972 | 44.2019 | 12.4% | 0.100 | 0.050 |
| Peak #18 | 24.284 | 3.66220 | 50.3296 | 14.1% | 0.124 | 0.062 |
| Peak #19 | 24.453 | 3.63726 | 40.2399 | 11.3% | 0.124 | 0.062 |
| Peak #20 | 25.058 | 3.55083 | 25.0506 | 7.0% | 0.100 | 0.050 |
| Peak #21 | 25.391 | 3.50502 | 40.0397 | 11.2% | 0.249 | 0.125 |
| Peak #22 | 25.791 | 3.45164 | 18.464 | 5.2% | 0.112 | 0.056 |
| Peak #23 | 26.964 | 3.30408 | 45.8814 | 12.9% | 0.114 | 0.057 |
| Peak #24 | 27.646 | 3.22408 | 17.5953 | 4.9% | 0.136 | 0.068 |
| Peak #25 | 28.665 | 3.11176 | 26.6018 | 7.5% | 0.100 | 0.050 |
| Peak #26 | 28.979 | 3.07872 | 56.7194 | 15.9% | 0.100 | 0.050 |
| Peak #27 | 31.387 | 2.84782 | 14.14 | 4.0% | 0.173 | 0.087 |
| Peak #28 | 32.552 | 2.74844 | 12.4754 | 3.5% | 0.167 | 0.083 |
| Peak #29 | 35.005 | 2.56127 | 29.6109 | 8.3% | 0.100 | 0.050 |
| Peak #30 | 35.683 | 2.51413 | 22.3212 | 6.3% | 0.100 | 0.050 |
| Peak #31 | 36.095 | 2.48641 | 26.2143 | 7.4% | 0.239 | 0.120 |
| Peak #32 | 36.496 | 2.46002 | 23.5539 | 6.6% | 0.100 | 0.050 |

### Observed Peaks for Compound 1, Form E:

| | | | Net In- | Rel. In- | | Width |
|---|---|---|---|---|---|---|
| Name | Angle/° | d Value/Å | tensity | tensity | FWHM | (low) |
| Peak #1 | 6.119 | 14.43315 | 3620.62 | 100.0% | 0.115 | 0.058 |
| Peak #2 | 6.959 | 12.69117 | 396.572 | 11.0% | 0.107 | 0.053 |
| Peak #3 | 7.350 | 12.01804 | 24.453 | 0.7% | 0.132 | 0.066 |
| Peak #4 | 8.610 | 10.26202 | 105.055 | 2.9% | 0.169 | 0.084 |
| Peak #5 | 9.670 | 9.13930 | 47.4516 | 1.3% | 0.100 | 0.050 |
| Peak #6 | 11.036 | 8.01063 | 28.9185 | 0.8% | 0.100 | 0.050 |
| Peak #7 | 11.706 | 7.55379 | 207.354 | 5.7% | 0.114 | 0.057 |
| Peak #8 | 12.369 | 7.15002 | 24.5847 | 0.7% | 0.229 | 0.115 |
| Peak #9 | 12.932 | 6.84018 | 63.7869 | 1.8% | 0.141 | 0.071 |
| Peak #10 | 13.708 | 6.45480 | 288.734 | 8.0% | 0.200 | 0.100 |
| Peak #11 | 15.025 | 5.89188 | 211.835 | 5.9% | 0.100 | 0.050 |
| Peak #12 | 16.008 | 5.53207 | 18.1912 | 0.5% | 0.100 | 0.050 |
| Peak #13 | 17.385 | 5.09693 | 229.835 | 6.3% | 0.202 | 0.101 |
| Peak #14 | 18.449 | 4.80534 | 93.2565 | 2.6% | 0.269 | 0.134 |
| Peak #15 | 18.715 | 4.73749 | 282.181 | 7.8% | 0.122 | 0.061 |
| Peak #16 | 19.047 | 4.65579 | 141.592 | 3.9% | 0.132 | 0.066 |
| Peak #17 | 19.533 | 4.54090 | 46.25 | 1.3% | 0.117 | 0.058 |
| Peak #18 | 19.810 | 4.47813 | 114.467 | 3.2% | 0.129 | 0.064 |
| Peak #19 | 20.660 | 4.29579 | 145.614 | 4.0% | 0.109 | 0.055 |
| Peak #20 | 21.321 | 4.16406 | 107.195 | 3.0% | 0.102 | 0.051 |
| Peak #21 | 22.251 | 3.99198 | 136.603 | 3.8% | 0.145 | 0.072 |
| Peak #22 | 22.676 | 3.91818 | 136.363 | 3.8% | 0.144 | 0.072 |
| Peak #23 | 24.424 | 3.64151 | 24.2272 | 0.7% | 0.160 | 0.080 |
| Peak #24 | 24.878 | 3.57608 | 169.782 | 4.7% | 0.104 | 0.052 |
| Peak #25 | 25.895 | 3.43799 | 128.082 | 3.5% | 0.124 | 0.062 |
| Peak #26 | 26.323 | 3.38301 | 52.2837 | 1.4% | 0.140 | 0.070 |
| Peak #27 | 27.000 | 3.29971 | 42.796 | 1.2% | 0.144 | 0.072 |
| Peak #28 | 27.800 | 3.20649 | 69.3307 | 1.9% | 0.141 | 0.070 |
| Peak #29 | 28.582 | 3.12056 | 23.493 | 0.6% | 0.100 | 0.050 |
| Peak #30 | 29.815 | 2.99428 | 13.875 | 0.4% | 0.193 | 0.097 |
| Peak #31 | 30.836 | 2.89738 | 77.9657 | 2.2% | 0.100 | 0.050 |
| Peak #32 | 31.887 | 2.80423 | 13.4285 | 0.4% | 0.193 | 0.097 |
| Peak #33 | 34.605 | 2.58999 | 25.4009 | 0.7% | 0.100 | 0.050 |
| Peak #34 | 37.298 | 2.40892 | 15.8784 | 0.4% | 0.161 | 0.080 |

### Observed Peaks for Compound 2, Form a:

| °2θ | d space (Å) | Intensity (%) |
|---|---|---|
| 6.58 ± 0.20 | 13.422 ± 0.408 | 100 |
| 7.27 ± 0.20 | 12.150 ± 0.334 | 26 |
| 9.24 ± 0.20 | 9.563 ± 0.207 | 6 |
| 10.38 ± 0.20 | 8.515 ± 0.164 | 5 |
| 12.08 ± 0.20 | 7.321 ± 0.121 | 4 |
| 13.37 ± 0.20 | 6.617 ± 0.099 | 27 |
| 13.73 ± 0.20 | 6.444 ± 0.093 | 12 |
| 13.91 ± 0.20 | 6.361 ± 0.091 | 18 |
| 14.33 ± 0.20 | 6.176 ± 0.086 | 18 |
| 14.59 ± 0.20 | 6.066 ± 0.083 | 53 |
| 14.86 ± 0.20 | 5.957 ± 0.080 | 24 |
| 15.29 ± 0.20 | 5.790 ± 0.075 | 7 |
| 15.61 ± 0.20 | 5.672 ± 0.072 | 36 |
| 15.84 ± 0.20 | 5.590 ± 0.070 | 22 |
| 16.73 ± 0.20 | 5.296 ± 0.063 | 3 |
| 17.45 ± 0.20 | 5.078 ± 0.058 | 10 |
| 18.55 ± 0.20 | 4.779 ± 0.051 | 56 |
| 18.68 ± 0.20 | 4.746 ± 0.050 | 39 |
| 19.27 ± 0.20 | 4.602 ± 0.047 | 20 |
| 19.69 ± 0.20 | 4.505 ± 0.045 | 15 |
| 19.89 ± 0.20 | 4.460 ± 0.044 | 12 |
| 20.37 ± 0.20 | 4.356 ± 0.042 | 14 |
| 20.88 ± 0.20 | 4.251 ± 0.040 | 35 |
| 21.97 ± 0.20 | 4.042 ± 0.036 | 56 |
| 22.21 ± 0.20 | 3.999 ± 0.036 | 23 |
| 23.35 ± 0.20 | 3.807 ± 0.032 | 47 |
| 23.70 ± 0.20 | 3.751 ± 0.031 | 10 |
| 23.89 ± 0.20 | 3.722 ± 0.031 | 12 |
| 24.31 ± 0.20 | 3.658 ± 0.030 | 49 |
| 24.49 ± 0.20 | 3.632 ± 0.029 | 23 |
| 24.91 ± 0.20 | 3.572 ± 0.028 | 9 |
| 25.44 ± 0.20 | 3.498 ± 0.027 | 56 |
| 25.88 ± 0.20 | 3.440 ± 0.026 | 5 |
| 26.16 ± 0.20 | 3.404 ± 0.026 | 7 |
| 26.48 ± 0.20 | 3.363 ± 0.025 | 9 |
| 26.94 ± 0.20 | 3.307 ± 0.024 | 35 |
| 27.67 ± 0.20 | 3.221 ± 0.023 | 6 |
| 27.99 ± 0.20 | 3.185 ± 0.022 | 19 |
| 28.51 ± 0.20 | 3.128 ± 0.021 | 16 |
| 29.25 ± 0.20 | 3.051 ± 0.020 | 5 |
| 29.50 ± 0.20 | 3.025 ± 0.020 | 11 |
| 30.05 ± 0.20 | 2.971 ± 0.019 | 3 |
| 30.39 ± 0.20 | 2.939 ± 0.019 | 6 |
| 30.89 ± 0.20 | 2.892 ± 0.018 | 11 |

### Observed Peaks for Compound 3, Form a:

| °2θ | d space (Å) | Intensity (%) |
|---|---|---|
| 6.81 ± 0.20 | 12.974 ± 0.381 | 100 |
| 7.24 ± 0.20 | 12.195 ± 0.336 | 25 |
| 9.53 ± 0.20 | 9.269 ± 0.194 | 5 |
| 10.37 ± 0.20 | 8.523 ± 0.164 | 8 |
| 12.19 ± 0.20 | 7.252 ± 0.118 | 8 |
| 13.57 ± 0.20 | 6.519 ± 0.096 | 28 |
| 14.06 ± 0.20 | 6.294 ± 0.089 | 15 |
| 14.53 ± 0.20 | 6.091 ± 0.083 | 75 |
| 15.12 ± 0.20 | 5.855 ± 0.077 | 34 |
| 15.67 ± 0.20 | 5.651 ± 0.072 | 28 |
| 16.03 ± 0.20 | 5.525 ± 0.069 | 20 |
| 17.58 ± 0.20 | 5.040 ± 0.057 | 10 |
| 18.75 ± 0.20 | 4.730 ± 0.050 | 30 |
| 19.13 ± 0.20 | 4.635 ± 0.048 | 58 |
| 19.50 ± 0.20 | 4.549 ± 0.046 | 13 |
| 19.90 ± 0.20 | 4.457 ± 0.044 | 17 |
| 20.58 ± 0.20 | 4.313 ± 0.041 | 20 |
| 20.83 ± 0.20 | 4.260 ± 0.040 | 31 |
| 21.23 ± 0.20 | 4.181 ± 0.039 | 10 |
| 21.56 ± 0.20 | 4.119 ± 0.038 | 20 |
| 21.88 ± 0.20 | 4.059 ± 0.037 | 11 |
| 22.45 ± 0.20 | 3.957 ± 0.035 | 62 |
| 23.56 ± 0.20 | 3.773 ± 0.032 | 52 |
| 24.24 ± 0.20 | 3.669 ± 0.030 | 29 |
| 24.59 ± 0.20 | 3.618 ± 0.029 | 62 |
| 25.76 ± 0.20 | 3.455 ± 0.026 | 63 |
| 26.32 ± 0.20 | 3.383 ± 0.025 | 14 |
| 27.34 ± 0.20 | 3.259 ± 0.023 | 43 |
| 28.88 ± 0.20 | 3.089 ± 0.021 | 23 |
| 29.13 ± 0.20 | 3.063 ± 0.021 | 20 |

### Observed Peaks for Compound 3, Form B:

| | °2θ | | Net In- | Rel. In- | | Width |
|---|---|---|---|---|---|---|
| Name | (±0.20) | d Value/Å | tensity | tensity | FWHM | (low) |
| Peak #1 | 6.662 | 13.25760 | 272 | 100% | 0.13 | 0.07 |
| Peak #2 | 7.207 | 12.25571 | 37.8 | 13.9% | 0.11 | 0.06 |
| Peak #3 | 10.287 | 8.59218 | 24.4 | 9.0% | 0.10 | 0.05 |
| Peak #4 | 13.276 | 6.66350 | 47.2 | 17.4% | 0.25 | 0.13 |
| Peak #5 | 14.572 | 6.07396 | 256 | 94.2% | 0.14 | 0.07 |
| Peak #6 | 15.875 | 5.57828 | 100 | 36.9% | 0.15 | 0.08 |
| Peak #7 | 19.011 | 4.66457 | 228 | 84.0% | 0.14 | 0.07 |
| Peak #8 | 20.305 | 4.36999 | 124 | 45.7% | 0.23 | 0.12 |
| Peak #9 | 20.817 | 4.26371 | 130 | 47.8% | 0.20 | 0.10 |
| Peak #10 | 22.270 | 3.98868 | 96.2 | 35.4% | 0.25 | 0.13 |
| Peak #11 | 24.026 | 3.70092 | 236 | 87.0% | 0.31 | 0.16 |
| Peak #12 | 25.658 | 3.46914 | 144 | 52.8% | 0.10 | 0.05 |
| Peak #13 | 28.795 | 3.09799 | 187 | 68.8% | 0.21 | 0.11 |
| Peak #14 | 34.526 | 2.59572 | 29.2 | 10.7% | 0.14 | 0.07 |
| Peak #15 | 36.167 | 2.48163 | 32.4 | 11.9% | 0.19 | 0.10 |

### Observed Peaks for Compound 3, Form C:

| | °2θ | | Net In- | Rel. In- | | Width |
|---|---|---|---|---|---|---|
| Name | (±0.20) | d Value/Å | tensity | tensity | FWHM | (low) |
| Peak #1 | 4.605 | 19.17483 | 12.8009 | 4.4% | 0.108 | 0.054 |
| Peak #2 | 6.629 | 13.32409 | 291.742 | 100.0% | 0.100 | 0.050 |
| Peak #3 | 7.210 | 12.25028 | 36.5449 | 12.5% | 0.100 | 0.050 |
| Peak #4 | 10.233 | 8.63775 | 15.5556 | 5.3% | 0.158 | 0.079 |
| Peak #5 | 11.934 | 7.40969 | 25.1272 | 8.6% | 0.100 | 0.050 |
| Peak #6 | 13.050 | 6.77851 | 16.3485 | 5.6% | 0.100 | 0.050 |
| Peak #7 | 13.412 | 6.59645 | 31.975 | 11.0% | 0.165 | 0.082 |
| Peak #8 | 14.658 | 6.03847 | 152.631 | 52.3% | 0.100 | 0.050 |
| Peak #9 | 14.947 | 5.92213 | 51.3123 | 17.6% | 0.290 | 0.145 |
| Peak #10 | 15.839 | 5.59083 | 129.364 | 44.3% | 0.101 | 0.050 |
| Peak #11 | 17.577 | 5.04161 | 73.5241 | 25.2% | 0.100 | 0.050 |
| Peak #12 | 18.684 | 4.74546 | 11.3791 | 3.9% | 0.131 | 0.066 |
| Peak #13 | 19.100 | 4.64287 | 118.914 | 40.8% | 0.100 | 0.050 |
| Peak #14 | 20.231 | 4.38595 | 66.3676 | 22.7% | 0.156 | 0.078 |
| Peak #15 | 20.426 | 4.34440 | 29.2578 | 10.0% | 0.156 | 0.078 |
| Peak #16 | 20.776 | 4.27194 | 226.055 | 77.5% | 0.100 | 0.050 |
| Peak #17 | 21.166 | 4.19418 | 38.1845 | 13.1% | 0.140 | 0.070 |
| Peak #18 | 21.712 | 4.08999 | 23.4458 | 8.0% | 0.107 | 0.053 |
| Peak #19 | 22.106 | 4.01781 | 246.192 | 84.4% | 0.100 | 0.050 |
| Peak #20 | 22.332 | 3.97776 | 74.8062 | 25.6% | 0.100 | 0.050 |
| Peak #21 | 23.425 | 3.79458 | 26.8884 | 9.2% | 0.149 | 0.074 |
| Peak #22 | 23.693 | 3.75220 | 40.2916 | 13.8% | 0.159 | 0.080 |
| Peak #23 | 24.143 | 3.68326 | 120.413 | 41.3% | 0.126 | 0.063 |
| Peak #24 | 24.954 | 3.56548 | 19.1769 | 6.6% | 0.202 | 0.101 |
| Peak #25 | 25.599 | 3.47707 | 127.804 | 43.8% | 0.101 | 0.050 |
| Peak #26 | 26.231 | 3.39467 | 31.7951 | 10.9% | 0.139 | 0.069 |
| Peak #27 | 27.155 | 3.28118 | 16.3494 | 5.6% | 0.111 | 0.055 |
| Peak #28 | 27.162 | 3.28034 | 18.2685 | 6.3% | 0.111 | 0.055 |
| Peak #29 | 27.655 | 3.22300 | 26.2761 | 9.0% | 0.136 | 0.068 |
| Peak #30 | 28.745 | 3.10323 | 112.119 | 38.4% | 0.152 | 0.076 |
| Peak #31 | 31.093 | 2.87408 | 20.3228 | 7.0% | 0.115 | 0.057 |
| Peak #32 | 31.593 | 2.82965 | 13.0664 | 4.5% | 0.242 | 0.121 |
| Peak #33 | 32.102 | 2.78595 | 15.1196 | 5.2% | 0.129 | 0.065 |
| Peak #34 | 33.816 | 2.64861 | 16.137 | 5.5% | 0.100 | 0.050 |
| Peak #35 | 37.608 | 2.38978 | 16.6469 | 5.7% | 0.157 | 0.079 |

### Observed Peaks for Compound 3, Form D (Compound of Formula II):

| | °2θ | | Net In- | Rel. In- | | Width |
|---|---|---|---|---|---|---|
| Name | (±0.20) | d Value/Å | tensity | tensity | FWHM | (low) |
| Peak #1 | 6.659 | 13.26296 | 723.131 | 100.0% | 0.109 | 0.054 |
| Peak #2 | 10.223 | 8.64626 | 19.2646 | 2.7% | 0.100 | 0.050 |
| Peak #3 | 13.475 | 6.56554 | 29.6582 | 4.1% | 0.125 | 0.063 |
| Peak #4 | 15.908 | 5.56650 | 228.855 | 31.6% | 0.100 | 0.050 |
| Peak #5 | 19.008 | 4.66508 | 15.5229 | 2.1% | 0.163 | 0.082 |
| Peak #6 | 20.194 | 4.39373 | 85.8765 | 11.9% | 0.100 | 0.050 |
| Peak #7 | 20.455 | 4.33826 | 178.291 | 24.7% | 0.230 | 0.115 |
| Peak #8 | 20.743 | 4.27870 | 144.739 | 20.0% | 0.100 | 0.050 |
| Peak #9 | 22.315 | 3.98079 | 357.717 | 49.5% | 0.109 | 0.055 |
| Peak #10 | 23.458 | 3.78929 | 23.658 | 3.3% | 0.100 | 0.050 |
| Peak #11 | 24.086 | 3.69195 | 20.3988 | 2.8% | 0.115 | 0.057 |
| Peak #12 | 25.599 | 3.47696 | 16.1779 | 2.2% | 0.109 | 0.055 |
| Peak #13 | 26.188 | 3.40009 | 26.3245 | 3.6% | 0.100 | 0.050 |
| Peak #14 | 27.182 | 3.27801 | 12.9493 | 1.8% | 0.225 | 0.113 |
| Peak #15 | 28.732 | 3.10462 | 105.397 | 14.6% | 0.100 | 0.050 |
| Peak #16 | 29.035 | 3.07285 | 235.643 | 32.6% | 0.116 | 0.058 |
| Peak #17 | 32.288 | 2.77032 | 9.72017 | 1.3% | 0.100 | 0.050 |
| Peak #18 | 36.076 | 2.48770 | 22.9181 | 3.2% | 0.170 | 0.085 |

### Observed Peaks for Compound 3, Form E:

| | °2θ | | Net In- | Rel. In- | | Width |
|---|---|---|---|---|---|---|
| Name | (±0.20) | d Value/Å | tensity | tensity | FWHM | (low) |
| Peak #1 | 6.640 | 13.30092 | 157.091 | 100.0% | 0.203 | 0.102 |
| Peak #2 | 8.408 | 10.50773 | 54.1509 | 34.5% | 0.148 | 0.074 |
| Peak #3 | 10.770 | 8.20779 | 78.8685 | 50.2% | 0.168 | 0.084 |
| Peak #4 | 11.817 | 7.48313 | 110.142 | 70.1% | 0.100 | 0.050 |
| Peak #5 | 12.159 | 7.27335 | 102.773 | 65.4% | 0.100 | 0.050 |
| Peak #6 | 12.731 | 6.94784 | 85.3914 | 54.4% | 0.213 | 0.107 |
| Peak #7 | 13.288 | 6.65777 | 17.4415 | 11.1% | 0.178 | 0.089 |
| Peak #8 | 15.089 | 5.86671 | 29.5253 | 18.8% | 0.167 | 0.084 |
| Peak #9 | 15.986 | 5.53973 | 60.3912 | 38.4% | 0.170 | 0.085 |
| Peak #10 | 16.446 | 5.38559 | 99.6936 | 63.5% | 0.216 | 0.108 |
| Peak #11 | 17.041 | 5.19896 | 48.8203 | 31.1% | 0.202 | 0.101 |
| Peak #12 | 18.011 | 4.92125 | 140.782 | 89.6% | 0.198 | 0.099 |
| Peak #13 | 18.689 | 4.74422 | 92.5387 | 58.9% | 0.170 | 0.085 |
| Peak #14 | 19.299 | 4.59541 | 20.3309 | 12.9% | 0.181 | 0.091 |
| Peak #15 | 21.383 | 4.15213 | 79.7781 | 50.8% | 0.201 | 0.100 |
| Peak #16 | 21.862 | 4.06219 | 35.8985 | 22.9% | 0.116 | 0.058 |
| Peak #17 | 22.773 | 3.90174 | 23.4161 | 14.9% | 0.116 | 0.058 |
| Peak #18 | 23.681 | 3.75407 | 55.4195 | 35.3% | 0.233 | 0.117 |
| Peak #19 | 24.649 | 3.60878 | 43.1416 | 27.5% | 0.329 | 0.165 |
| Peak #20 | 26.241 | 3.39340 | 30.7285 | 19.6% | 0.164 | 0.082 |

### Observed Peaks for Compound 4, Form a (Compound of Formula I):

| °2θ | d space (Å) | Intensity (%) |
|---|---|---|
| 8.26 ± 0.20 | 10.696 ± 0.259 | 74 |
| 9.41 ± 0.20 | 9.391 ± 0.199 | 20 |
| 10.08 ± 0.20 | 8.768 ± 0.174 | 51 |
| 10.35 ± 0.20 | 8.540 ± 0.165 | 23 |
| 10.94 ± 0.20 | 8.081 ± 0.147 | 19 |
| 11.34 ± 0.20 | 7.797 ± 0.137 | 16 |
| 12.03 ± 0.20 | 7.351 ± 0.122 | 11 |
| 12.29 ± 0.20 | 7.196 ± 0.117 | 79 |
| 12.66 ± 0.20 | 6.987 ± 0.110 | 10 |
| 13.75 ± 0.20 | 6.435 ± 0.093 | 69 |
| 14.96 ± 0.20 | 5.917 ± 0.079 | 23 |
| 15.08 ± 0.20 | 5.870 ± 0.077 | 19 |
| 15.57 ± 0.20 | 5.687 ± 0.073 | 12 |
| 15.77 ± 0.20 | 5.615 ± 0.071 | 52 |
| 16.58 ± 0.20 | 5.342 ± 0.064 | 15 |
| 17.07 ± 0.20 | 5.190 ± 0.060 | 57 |
| 17.76 ± 0.20 | 4.990 ± 0.056 | 100 |
| 18.20 ± 0.20 | 4.870 ± 0.053 | 28 |
| 18.44 ± 0.20 | 4.808 ± 0.052 | 56 |
| 18.93 ± 0.20 | 4.684 ± 0.049 | 16 |
| 19.22 ± 0.20 | 4.614 ± 0.048 | 13 |
| 19.65 ± 0.20 | 4.514 ± 0.045 | 45 |
| 20.18 ± 0.20 | 4.397 ± 0.043 | 5 |
| 20.78 ± 0.20 | 4.271 ± 0.041 | 21 |
| 21.63 ± 0.20 | 4.105 ± 0.038 | 58 |
| 22.08 ± 0.20 | 4.023 ± 0.036 | 17 |
| 22.80 ± 0.20 | 3.897 ± 0.034 | 90 |
| 23.16 ± 0.20 | 3.837 ± 0.033 | 69 |
| 23.38 ± 0.20 | 3.802 ± 0.032 | 91 |
| 23.73 ± 0.20 | 3.746 ± 0.031 | 30 |
| 24.14 ± 0.20 | 3.684 ± 0.030 | 49 |
| 24.44 ± 0.20 | 3.639 ± 0.029 | 17 |
| 24.73 ± 0.20 | 3.597 ± 0.029 | 39 |
| 25.25 ± 0.20 | 3.524 ± 0.027 | 36 |
| 25.53 ± 0.20 | 3.486 ± 0.027 | 32 |
| 25.92 ± 0.20 | 3.435 ± 0.026 | 30 |
| 26.28 ± 0.20 | 3.388 ± 0.025 | 11 |
| 26.81 ± 0.20 | 3.323 ± 0.024 | 21 |
| 27.22 ± 0.20 | 3.273 ± 0.024 | 7 |
| 27.62 ± 0.20 | 3.227 ± 0.023 | 22 |
| 28.11 ± 0.20 | 3.172 ± 0.022 | 13 |
| 28.52 ± 0.20 | 3.127 ± 0.021 | 6 |
| 28.80 ± 0.20 | 3.097 ± 0.021 | 10 |
| 29.44 ± 0.20 | 3.032 ± 0.020 | 20 |
| 29.90 ± 0.20 | 2.986 ± 0.020 | 31 |
| 30.46 ± 0.20 | 2.932 ± 0.019 | 11 |
| 30.64 ± 0.20 | 2.915 ± 0.019 | 13 |

### Observed Peaks for Compound 4, Form B

| | °2θ | | Net In- | Rel. In- | | Width |
|---|---|---|---|---|---|---|
| Name | (±0.20) | d Value/Å | tensity | tensity | FWHM | (low) |
| Peak #1 | 6.832 | 12.92751 | 590.404 | 76.7% | 0.104 | 0.052 |
| Peak #2 | 9.206 | 9.59848 | 80.5638 | 10.5% | 0.100 | 0.050 |
| Peak #3 | 9.805 | 9.01349 | 66.3317 | 8.6% | 0.100 | 0.050 |
| Peak #4 | 10.750 | 8.22313 | 15.3015 | 2.0% | 0.100 | 0.050 |
| Peak #5 | 11.760 | 7.51894 | 71.5626 | 9.3% | 0.130 | 0.065 |
| Peak #6 | 12.235 | 7.22852 | 163.981 | 21.3% | 0.151 | 0.076 |
| Peak #7 | 13.748 | 6.43578 | 70.4153 | 9.1% | 0.100 | 0.050 |
| Peak #8 | 14.518 | 6.09632 | 22.1022 | 2.9% | 0.150 | 0.075 |
| Peak #9 | 14.784 | 5.98741 | 29.0078 | 3.8% | 0.154 | 0.077 |
| Peak #10 | 15.921 | 5.56213 | 213.212 | 27.7% | 0.100 | 0.050 |
| Peak #11 | 17.099 | 5.18146 | 113.516 | 14.7% | 0.167 | 0.083 |
| Peak #12 | 17.415 | 5.08811 | 191.955 | 24.9% | 0.198 | 0.099 |
| Peak #13 | 18.039 | 4.91362 | 138.666 | 18.0% | 0.113 | 0.057 |
| Peak #14 | 18.455 | 4.80375 | 203.076 | 26.4% | 0.223 | 0.111 |
| Peak #15 | 19.089 | 4.64556 | 149.154 | 19.4% | 0.125 | 0.062 |
| Peak #16 | 20.503 | 4.32824 | 22.0967 | 2.9% | 0.121 | 0.061 |
| Peak #17 | 21.074 | 4.21219 | 48.7253 | 6.3% | 0.155 | 0.078 |
| Peak #18 | 22.351 | 3.97446 | 184.143 | 23.9% | 0.150 | 0.075 |
| Peak #19 | 22.831 | 3.89194 | 770.124 | 100.0% | 0.122 | 0.061 |
| Peak #20 | 23.135 | 3.84152 | 146.142 | 19.0% | 0.232 | 0.116 |
| Peak #21 | 23.682 | 3.75390 | 319.89 | 41.5% | 0.160 | 0.080 |
| Peak #22 | 24.804 | 3.58667 | 89.1716 | 11.6% | 0.172 | 0.086 |
| Peak #23 | 26.070 | 3.41523 | 585.815 | 76.1% | 0.133 | 0.067 |
| Peak #24 | 26.590 | 3.34968 | 110.941 | 14.4% | 0.166 | 0.083 |
| Peak #25 | 27.787 | 3.20803 | 156.931 | 20.4% | 0.180 | 0.090 |
| Peak #26 | 28.094 | 3.17361 | 149.203 | 19.4% | 0.100 | 0.050 |
| Peak #27 | 28.779 | 3.09967 | 22.2874 | 2.9% | 0.144 | 0.072 |
| Peak #28 | 29.656 | 3.00999 | 22.1676 | 2.9% | 0.150 | 0.075 |
| Peak #29 | 29.992 | 2.97703 | 38.6272 | 5.0% | 0.185 | 0.092 |
| Peak #30 | 31.212 | 2.86337 | 62.6044 | 8.1% | 0.100 | 0.050 |
| Peak #31 | 31.729 | 2.81789 | 212.994 | 27.7% | 0.208 | 0.104 |
| Peak #32 | 33.333 | 2.68581 | 32.9444 | 4.3% | 0.163 | 0.082 |
| Peak #33 | 37.698 | 2.38426 | 28.2173 | 3.7% | 0.100 | 0.050 |

### Observed Peaks for Compound 4, Form C

| | °2θ | | Net In- | Rel. In- | | Width |
|---|---|---|---|---|---|---|
| Name | (±0.20) | d Value/Å | tensity | tensity | FWHM | (low) |
| Peak #1 | 5.344 | 16.52307 | 61.5646 | 34.6% | 0.100 | 0.050 |
| Peak #2 | 6.915 | 12.77295 | 96.9398 | 54.4% | 0.100 | 0.050 |
| Peak #3 | 7.946 | 11.11806 | 15.5793 | 8.7% | 0.167 | 0.084 |
| Peak #4 | 8.347 | 10.58488 | 36.7831 | 20.7% | 0.189 | 0.095 |
| Peak #5 | 9.092 | 9.71864 | 66.7444 | 37.5% | 0.105 | 0.052 |
| Peak #6 | 9.913 | 8.91601 | 56.8249 | 31.9% | 0.112 | 0.056 |
| Peak #7 | 10.170 | 8.69112 | 52.8897 | 29.7% | 0.217 | 0.108 |
| Peak #8 | 10.742 | 8.22937 | 82.2962 | 46.2% | 0.124 | 0.062 |
| Peak #9 | 11.839 | 7.46896 | 83.4558 | 46.9% | 0.139 | 0.070 |
| Peak #10 | 12.274 | 7.20530 | 26.6843 | 15.0% | 0.180 | 0.090 |
| Peak #11 | 13.364 | 6.62020 | 78.9947 | 44.4% | 0.107 | 0.053 |
| Peak #12 | 15.152 | 5.84267 | 178.095 | 100.0% | 0.153 | 0.076 |
| Peak #13 | 16.031 | 5.52429 | 169.006 | 94.9% | 0.125 | 0.062 |
| Peak #14 | 17.637 | 5.02469 | 117.206 | 65.8% | 0.215 | 0.108 |
| Peak #15 | 18.308 | 4.84190 | 72.531 | 40.7% | 0.190 | 0.095 |
| Peak #16 | 19.262 | 4.60423 | 102.827 | 57.7% | 0.171 | 0.085 |
| Peak #17 | 20.487 | 4.33156 | 111.087 | 62.4% | 0.207 | 0.103 |
| Peak #18 | 21.434 | 4.14225 | 65.9749 | 37.0% | 0.234 | 0.117 |
| Peak #19 | 22.089 | 4.02101 | 100.927 | 56.7% | 0.165 | 0.082 |
| Peak #20 | 22.982 | 3.86673 | 112.666 | 63.3% | 0.100 | 0.050 |
| Peak #21 | 23.288 | 3.81665 | 105.309 | 59.1% | 0.272 | 0.136 |
| Peak #22 | 23.943 | 3.71368 | 104.229 | 58.5% | 0.169 | 0.085 |
| Peak #23 | 26.249 | 3.39238 | 16.3819 | 9.2% | 0.100 | 0.050 |
| Peak #24 | 26.886 | 3.31342 | 38.4834 | 21.6% | 0.100 | 0.050 |
| Peak #25 | 27.623 | 3.22673 | 27.5541 | 15.5% | 0.229 | 0.114 |
| Peak #26 | 27.866 | 3.19913 | 50.6951 | 28.5% | 0.177 | 0.089 |

### Observed Peaks for Compound 4, Form D

| | °2θ | | Net In- | Rel. In- | | Width |
|---|---|---|---|---|---|---|
| Name | (±0.20) | d Value/Å | tensity | tensity | FWHM | (low) |
| Peak #1 | 8.217 | 10.75168 | 179.114 | 33.8% | 0.126 | 0.063 |
| Peak #2 | 9.824 | 8.99650 | 174.383 | 32.9% | 0.180 | 0.090 |
| Peak #3 | 11.144 | 7.93331 | 27.5397 | 5.2% | 0.116 | 0.058 |
| Peak #4 | 12.205 | 7.24609 | 65.8919 | 12.4% | 0.115 | 0.058 |
| Peak #5 | 13.277 | 6.66309 | 86.8529 | 16.4% | 0.181 | 0.091 |
| Peak #6 | 13.658 | 6.47843 | 89.8375 | 16.9% | 0.135 | 0.068 |
| Peak #7 | 14.152 | 6.25297 | 40.0042 | 7.5% | 0.199 | 0.100 |
| Peak #8 | 14.998 | 5.90234 | 90.8684 | 17.1% | 0.169 | 0.085 |
| Peak #9 | 15.708 | 5.63709 | 19.2496 | 3.6% | 0.100 | 0.050 |
| Peak #10 | 16.580 | 5.34249 | 65.5611 | 12.4% | 0.131 | 0.066 |
| Peak #11 | 17.062 | 5.19275 | 158.726 | 29.9% | 0.210 | 0.105 |
| Peak #12 | 17.562 | 5.04582 | 530.699 | 100.0% | 0.162 | 0.081 |
| Peak #13 | 18.357 | 4.82919 | 59.7555 | 11.3% | 0.189 | 0.095 |
| Peak #14 | 19.000 | 4.66707 | 82.3058 | 15.5% | 0.169 | 0.084 |
| Peak #15 | 19.565 | 4.53363 | 30.8627 | 5.8% | 0.197 | 0.098 |
| Peak #16 | 20.130 | 4.40757 | 20.8451 | 3.9% | 0.135 | 0.067 |
| Peak #17 | 21.285 | 4.17102 | 20.8717 | 3.9% | 0.100 | 0.050 |
| Peak #18 | 21.543 | 4.12159 | 61.7404 | 11.6% | 0.136 | 0.068 |
| Peak #19 | 22.035 | 4.03078 | 190.177 | 35.8% | 0.162 | 0.081 |
| Peak #20 | 23.237 | 3.82490 | 306.957 | 57.8% | 0.387 | 0.193 |
| Peak #21 | 24.133 | 3.68482 | 142.263 | 26.8% | 0.247 | 0.124 |
| Peak #22 | 24.610 | 3.61445 | 73.4227 | 13.8% | 0.100 | 0.050 |
| Peak #23 | 25.237 | 3.52610 | 132.319 | 24.9% | 0.239 | 0.120 |
| Peak #24 | 25.850 | 3.44387 | 165.577 | 31.2% | 0.161 | 0.080 |
| Peak #25 | 26.700 | 3.33611 | 45.7194 | 8.6% | 0.119 | 0.060 |
| Peak #26 | 27.505 | 3.24021 | 33.0488 | 6.2% | 0.180 | 0.090 |
| Peak #27 | 28.669 | 3.11130 | 83.2759 | 15.7% | 0.138 | 0.069 |
| Peak #28 | 29.311 | 3.04456 | 48.8384 | 9.2% | 0.173 | 0.087 |
| Peak #29 | 29.844 | 2.99143 | 50.9785 | 9.6% | 0.227 | 0.114 |
| Peak #30 | 30.057 | 2.97074 | 59.9843 | 11.3% | 0.209 | 0.104 |
| Peak #31 | 30.713 | 2.90874 | 52.6201 | 9.9% | 0.226 | 0.113 |
| Peak #32 | 31.441 | 2.84302 | 29.3548 | 5.5% | 0.138 | 0.069 |
| Peak #33 | 33.135 | 2.70140 | 38.3054 | 7.2% | 0.169 | 0.084 |
| Peak #34 | 33.631 | 2.66269 | 33.1537 | 6.2% | 0.236 | 0.118 |
| Peak #35 | 34.522 | 2.59602 | 42.3866 | 8.0% | 0.100 | 0.050 |
| Peak #36 | 37.696 | 2.38440 | 12.6906 | 2.4% | 0.141 | 0.070 |

### Observed Peaks for Compound 4, Form E

| | °2θ | | Net In- | Rel. In- | | Width |
|---|---|---|---|---|---|---|
| Name | (±0.20) | d Value/Å | tensity | tensity | FWHM | (low) |
| Peak #1 | 4.237 | 20.83746 | 327.509 | 16.9% | 0.100 | 0.050 |
| Peak #2 | 6.037 | 14.62837 | 16.765 | 0.9% | 0.173 | 0.087 |
| Peak #3 | 6.500 | 13.58730 | 59.1341 | 3.1% | 0.100 | 0.050 |
| Peak #4 | 7.326 | 12.05672 | 1845.76 | 95.3% | 0.100 | 0.050 |
| Peak #5 | 8.330 | 10.60604 | 44.4048 | 2.3% | 0.269 | 0.134 |
| Peak #6 | 8.685 | 10.17321 | 846.563 | 43.7% | 0.100 | 0.050 |
| Peak #7 | 10.188 | 8.67524 | 873.76 | 45.1% | 0.100 | 0.050 |
| Peak #8 | 11.115 | 7.95393 | 50.0088 | 2.6% | 0.100 | 0.050 |
| Peak #9 | 11.631 | 7.60252 | 245.105 | 12.7% | 0.100 | 0.050 |
| Peak #10 | 12.739 | 6.94321 | 96.2704 | 5.0% | 0.177 | 0.089 |
| Peak #11 | 13.138 | 6.73360 | 929.654 | 48.0% | 0.100 | 0.050 |
| Peak #12 | 13.372 | 6.61632 | 118.458 | 6.1% | 0.100 | 0.050 |
| Peak #13 | 13.900 | 6.36573 | 259.593 | 13.4% | 0.100 | 0.050 |
| Peak #14 | 14.157 | 6.25118 | 47.9129 | 2.5% | 0.100 | 0.050 |
| Peak #15 | 14.524 | 6.09397 | 106.848 | 5.5% | 0.292 | 0.146 |
| Peak #16 | 14.880 | 5.94869 | 1936.3 | 100.0% | 0.100 | 0.050 |
| Peak #17 | 15.534 | 5.69967 | 92.887 | 4.8% | 0.147 | 0.073 |
| Peak #18 | 15.706 | 5.63777 | 124.381 | 6.4% | 0.146 | 0.073 |
| Peak #19 | 16.513 | 5.36410 | 48.7606 | 2.5% | 0.100 | 0.050 |
| Peak #20 | 17.038 | 5.19992 | 46.5061 | 2.4% | 0.115 | 0.058 |
| Peak #21 | 17.344 | 5.10873 | 42.4958 | 2.2% | 0.222 | 0.111 |
| Peak #22 | 17.412 | 5.08914 | 65.101 | 3.4% | 0.222 | 0.111 |
| Peak #23 | 17.705 | 5.00543 | 133.136 | 6.9% | 0.100 | 0.050 |
| Peak #24 | 18.003 | 4.92332 | 668.383 | 34.5% | 0.100 | 0.050 |
| Peak #25 | 18.800 | 4.71626 | 53.9473 | 2.8% | 0.100 | 0.050 |
| Peak #26 | 19.334 | 4.58727 | 1003.92 | 51.8% | 0.100 | 0.050 |
| Peak #27 | 19.677 | 4.50806 | 400.118 | 20.7% | 0.161 | 0.080 |
| Peak #28 | 19.886 | 4.46117 | 283.195 | 14.6% | 0.161 | 0.081 |
| Peak #29 | 20.746 | 4.27805 | 103.879 | 5.4% | 0.165 | 0.083 |
| Peak #30 | 20.957 | 4.23549 | 201.93 | 10.4% | 0.145 | 0.073 |
| Peak #31 | 21.527 | 4.12461 | 292.686 | 15.1% | 0.100 | 0.050 |
| Peak #32 | 22.119 | 4.01555 | 917.13 | 47.4% | 0.100 | 0.050 |
| Peak #33 | 22.519 | 3.94515 | 391.197 | 20.2% | 0.100 | 0.050 |
| Peak #34 | 22.977 | 3.86745 | 521.781 | 26.9% | 0.100 | 0.050 |
| Peak #35 | 23.176 | 3.83478 | 68.82 | 3.6% | 0.100 | 0.050 |
| Peak #36 | 23.597 | 3.76732 | 408.02 | 21.1% | 0.139 | 0.070 |
| Peak #37 | 23.771 | 3.74004 | 482.264 | 24.9% | 0.139 | 0.069 |
| Peak #38 | 24.006 | 3.70397 | 192.008 | 9.9% | 0.190 | 0.095 |
| Peak #39 | 24.367 | 3.65001 | 235.696 | 12.2% | 0.100 | 0.050 |
| Peak #40 | 24.675 | 3.60514 | 19.505 | 1.0% | 0.224 | 0.112 |
| Peak #41 | 24.957 | 3.56494 | 77.7918 | 4.0% | 0.100 | 0.050 |
| Peak #42 | 25.320 | 3.51466 | 118.324 | 6.1% | 0.182 | 0.091 |
| Peak #43 | 25.844 | 3.44465 | 65.7982 | 3.4% | 0.100 | 0.050 |
| Peak #44 | 26.627 | 3.34510 | 422.073 | 21.8% | 0.100 | 0.050 |
| Peak #45 | 27.100 | 3.28770 | 32.7623 | 1.7% | 0.100 | 0.050 |
| Peak #46 | 27.921 | 3.19295 | 72.873 | 3.8% | 0.100 | 0.050 |
| Peak #47 | 28.390 | 3.14125 | 211.464 | 10.9% | 0.106 | 0.053 |
| Peak #48 | 29.371 | 3.03854 | 22.8549 | 1.2% | 0.186 | 0.093 |
| Peak #49 | 29.942 | 2.98185 | 19.6862 | 1.0% | 0.136 | 0.068 |
| Peak #50 | 30.229 | 2.95420 | 83.1716 | 4.3% | 0.100 | 0.050 |
| Peak #51 | 30.778 | 2.90278 | 43.5582 | 2.2% | 0.102 | 0.051 |
| Peak #52 | 31.031 | 2.87964 | 166.103 | 8.6% | 0.212 | 0.106 |
| Peak #53 | 31.227 | 2.86200 | 146.321 | 7.6% | 0.211 | 0.106 |
| Peak #54 | 31.679 | 2.82216 | 49.9042 | 2.6% | 0.107 | 0.054 |
| Peak #55 | 32.123 | 2.78422 | 20.3337 | 1.1% | 0.157 | 0.078 |
| Peak #56 | 32.279 | 2.77106 | 52.8824 | 2.7% | 0.157 | 0.078 |
| Peak #57 | 32.863 | 2.72315 | 125.654 | 6.5% | 0.108 | 0.054 |
| Peak #58 | 34.677 | 2.58479 | 27.0214 | 1.4% | 0.232 | 0.116 |
| Peak #59 | 34.973 | 2.56357 | 51.7705 | 2.7% | 0.260 | 0.130 |
| Peak #60 | 35.306 | 2.54016 | 31.5362 | 1.6% | 0.180 | 0.090 |
| Peak #61 | 36.917 | 2.43291 | 25.9675 | 1.3% | 0.100 | 0.050 |
| Peak #62 | 37.455 | 2.39916 | 29.1868 | 1.5% | 0.100 | 0.050 |

### Observed Peaks for Compound 4, Form F

| | °2θ | | Net In- | Rel. In- | | Width |
|---|---|---|---|---|---|---|
| Name | (±0.20) | d Value/Å | tensity | tensity | FWHM | (low) |
| Peak #1 | 7.905 | 11.17567 | 298.356 | 38.6% | 0.100 | 0.050 |
| Peak #2 | 10.142 | 8.71489 | 122.193 | 15.8% | 0.100 | 0.050 |
| Peak #3 | 10.991 | 8.04368 | 58.5755 | 7.6% | 0.125 | 0.063 |
| Peak #4 | 12.141 | 7.28427 | 508.606 | 65.8% | 0.100 | 0.050 |
| Peak #5 | 13.929 | 6.35265 | 88.6148 | 11.5% | 0.109 | 0.054 |
| Peak #6 | 15.961 | 5.54840 | 122.843 | 15.9% | 0.106 | 0.053 |
| Peak #7 | 16.631 | 5.32625 | 238.714 | 30.9% | 0.100 | 0.050 |
| Peak #8 | 17.212 | 5.14777 | 81.1416 | 10.5% | 0.100 | 0.050 |
| Peak #9 | 18.160 | 4.88113 | 79.338 | 10.3% | 0.291 | 0.146 |
| Peak #10 | 18.509 | 4.78977 | 91.8793 | 11.9% | 0.299 | 0.149 |
| Peak #11 | 18.958 | 4.67730 | 257.662 | 33.4% | 0.110 | 0.055 |
| Peak #12 | 19.975 | 4.44155 | 329.591 | 42.7% | 0.100 | 0.050 |
| Peak #13 | 20.578 | 4.31270 | 155.85 | 20.2% | 0.155 | 0.077 |
| Peak #14 | 22.112 | 4.01689 | 221.873 | 28.7% | 0.180 | 0.090 |
| Peak #15 | 22.547 | 3.94030 | 101.186 | 13.1% | 0.100 | 0.050 |
| Peak #16 | 23.387 | 3.80069 | 772.533 | 100.0% | 0.113 | 0.057 |
| Peak #17 | 24.082 | 3.69255 | 140.975 | 18.2% | 0.164 | 0.082 |
| Peak #18 | 24.602 | 3.61560 | 385.91 | 50.0% | 0.191 | 0.096 |
| Peak #19 | 24.896 | 3.57353 | 273.355 | 35.4% | 0.239 | 0.120 |
| Peak #20 | 25.270 | 3.52150 | 49.8223 | 6.4% | 0.129 | 0.064 |
| Peak #21 | 27.132 | 3.28390 | 54.5365 | 7.1% | 0.100 | 0.050 |
| Peak #22 | 27.880 | 3.19749 | 73.896 | 9.6% | 0.205 | 0.103 |
| Peak #23 | 28.173 | 3.16496 | 27.9818 | 3.6% | 0.220 | 0.110 |
| Peak #24 | 28.628 | 3.11569 | 123.638 | 16.0% | 0.104 | 0.052 |
| Peak #25 | 29.771 | 2.99855 | 71.6486 | 9.3% | 0.169 | 0.085 |
| Peak #26 | 38.376 | 2.34367 | 32.2089 | 4.2% | 0.198 | 0.099 |

### Observed Peaks for Compound 4, Form G

| | °2θ | | Net In- | Rel. In- | | Width |
|---|---|---|---|---|---|---|
| Name | (±0.20) | d Value/Å | tensity | tensity | FWHM | (low) |
| Peak #1 | 5.719 | 15.44181 | 392.925 | 17.6% | 0.115 | 0.058 |
| Peak #2 | 9.534 | 9.26921 | 31.6983 | 1.4% | 0.100 | 0.050 |
| Peak #3 | 11.518 | 7.67676 | 196.053 | 8.8% | 0.113 | 0.057 |
| Peak #4 | 12.010 | 7.36344 | 40.386 | 1.8% | 0.122 | 0.061 |
| Peak #5 | 13.485 | 6.56077 | 116.045 | 5.2% | 0.100 | 0.050 |
| Peak #6 | 13.967 | 6.33548 | 2230.26 | 100.0% | 0.110 | 0.055 |
| Peak #7 | 14.619 | 6.05438 | 157.808 | 7.1% | 0.167 | 0.084 |
| Peak #8 | 15.732 | 5.62842 | 161.283 | 7.2% | 0.122 | 0.061 |
| Peak #9 | 16.551 | 5.35163 | 48.1001 | 2.2% | 0.121 | 0.061 |
| Peak #10 | 17.340 | 5.11004 | 47.2155 | 2.1% | 0.147 | 0.073 |
| Peak #11 | 17.764 | 4.98903 | 48.4234 | 2.2% | 0.147 | 0.073 |
| Peak #12 | 18.192 | 4.87266 | 107.382 | 4.8% | 0.258 | 0.129 |
| Peak #13 | 18.503 | 4.79135 | 487.07 | 21.8% | 0.116 | 0.058 |
| Peak #14 | 19.242 | 4.60905 | 264.986 | 11.9% | 0.118 | 0.059 |
| Peak #15 | 19.834 | 4.47277 | 207.188 | 9.3% | 0.122 | 0.061 |
| Peak #16 | 20.852 | 4.25670 | 138.16 | 6.2% | 0.131 | 0.066 |
| Peak #17 | 21.750 | 4.08294 | 176.751 | 7.9% | 0.134 | 0.067 |
| Peak #18 | 22.439 | 3.95897 | 90.7817 | 4.1% | 0.118 | 0.059 |
| Peak #19 | 23.355 | 3.80583 | 266.306 | 11.9% | 0.269 | 0.134 |
| Peak #20 | 23.863 | 3.72597 | 261.28 | 11.7% | 0.158 | 0.079 |
| Peak #21 | 24.120 | 3.68676 | 120.39 | 5.4% | 0.100 | 0.050 |
| Peak #22 | 25.009 | 3.55766 | 137.18 | 6.2% | 0.175 | 0.087 |
| Peak #23 | 25.908 | 3.43622 | 57.6546 | 2.6% | 0.289 | 0.144 |
| Peak #24 | 26.190 | 3.39987 | 92.9679 | 4.2% | 0.206 | 0.103 |
| Peak #25 | 27.511 | 3.23954 | 52.3308 | 2.3% | 0.109 | 0.055 |
| Peak #26 | 27.913 | 3.19378 | 24.0504 | 1.1% | 0.119 | 0.059 |
| Peak #27 | 28.567 | 3.12212 | 47.6781 | 2.1% | 0.111 | 0.055 |
| Peak #28 | 29.197 | 3.05620 | 73.1107 | 3.3% | 0.173 | 0.087 |
| Peak #29 | 30.460 | 2.93232 | 69.1283 | 3.1% | 0.149 | 0.074 |
| Peak #30 | 31.799 | 2.81182 | 34.7015 | 1.6% | 0.188 | 0.094 |
| Peak #31 | 31.815 | 2.81048 | 43.3988 | 1.9% | 0.188 | 0.094 |
| Peak #32 | 32.733 | 2.73365 | 16.3371 | 0.7% | 0.168 | 0.084 |
| Peak #33 | 33.746 | 2.65387 | 52.8032 | 2.4% | 0.130 | 0.065 |
| Peak #34 | 34.329 | 2.61014 | 58.9774 | 2.6% | 0.100 | 0.050 |
| Peak #35 | 37.868 | 2.37396 | 42.1249 | 1.9% | 0.114 | 0.057 |

Some of the embodiments of the present description are described in the following items:

1. A compound of Formula II:

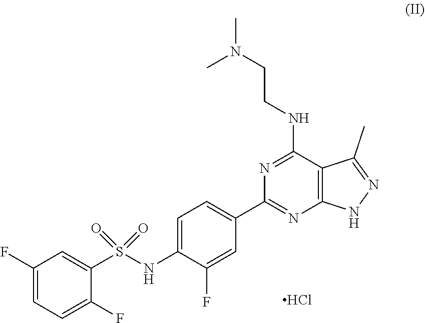 [SMILES: Cc1n[nH]c2nc(-c3ccc(NS(=O)(=O)c4cc(F)ccc4F)c(F)c3)nc(NCCN(C)C)c12 ; Cl]

2. The compound of item 1, which is crystalline and exhibits an X-ray powder diffraction (XRPD) pattern having characteristic peaks expressed in degrees 2θ (±0.2° 2θ) at 6.7, 22.3 and 29.0.

3. The compound of item 2, wherein the XRPD pattern further has characteristic peaks expressed in degrees 2θ (±0.2° 2θ) at 15.9, 20.5 and 20.7.

4. The compound of item 2 or 3, wherein the XRPD pattern further has characteristic peaks expressed in degrees 2θ (±0.2° 2θ) at 28.7, 20.2 and 13.5.

5. The compound of any one of items 2 to 4, wherein the XRPD pattern further has characteristic peaks expressed in degrees 2θ (±0.2° 2θ) at 26.2, 23.5 and 36.1.

6. The compound of any one of items 2 to 5, wherein the XRPD pattern further has characteristic peaks expressed in degrees 2θ (±0.2° 2θ) at 24.1, 10.2 and 25.6.

7. The compound of any one of items 2 to 6, wherein the XRPD pattern further has characteristic peaks expressed in degrees 2θ (±0.2° 2θ) at 19.0, 27.2 and 32.3.

8. The compound of any one of items 1 to 7, which is crystalline and has a Differential Scanning Calorimetry (DSC) thermogram that exhibits an endotherm having an onset of about 302° C.

9. The compound of item 1, which has an X-ray powder diffraction pattern substantially the same as shown in FIG. 29.

10. The compound of item 1, which is crystalline and exhibits an X-ray powder diffraction (XRPD) pattern having characteristic peaks expressed in degrees 2θ (±0.2° 2θ) at 6.8, 14.5 and 25.8.

11. The compound of item 10, wherein the XRPD pattern further has characteristic peaks expressed in degrees 2θ (±0.2° 2θ) at 24.6, 22.45 and 19.1.

12. The compound of item 10 or 11, wherein the XRPD pattern further has characteristic peaks expressed in degrees 2θ (±0.2° 2θ) at 23.6 and 27.3.

13. The compound of any one of items 10 to 12, wherein the XRPD pattern further has characteristic peaks expressed in degrees 2θ (±0.2° 2θ) at 15.1 and 20.8.

14. The compound of any one of items 1 and 10 to 13, which is crystalline and has a Differential Scanning Calorimetry (DSC) thermogram that exhibits an endotherm having an onset of about 308° C.

15. The compound of item 1, which has an X-ray powder diffraction pattern substantially the same as shown in FIG. 8.

16. The compound of item 1, which is crystalline and exhibits an X-ray powder diffraction (XRPD) pattern having characteristic peaks expressed in degrees 2θ (±0.2° 2θ) at 6.7, 14.6 and 24.0.

17. The compound of item 16, wherein the XRPD pattern further has characteristic peaks expressed in degrees 2θ (±0.2° 2θ) at 19.0, 28.8 and 25.7.

18. The compound of item 16 or 17, wherein the XRPD pattern further has characteristic peaks expressed in degrees 2θ (±0.2° 2θ) at 20.8 and 20.3.

19. The compound of any one of items 16 to 18, wherein the XRPD pattern further has characteristic peaks expressed in degrees 2θ (±0.2° 2θ) at 15.9 and 22.3.

20. The compound of any one of items 1 and 16 to 19, which is crystalline and has a Differential Scanning Calorimetry (DSC) thermogram that exhibits a first endotherm having an onset of about 50° C. and a second endotherm having an onset temperature of about 301° C.

21. The compound of item 1, which has an X-ray powder diffraction pattern substantially the same as shown in FIG. 25.

22. The compound of item 1, which is crystalline and exhibits an X-ray powder diffraction (XRPD) pattern having characteristic peaks expressed in degrees 2θ (±0.2° 2θ) at 6.6, 22.1 and 20.8.

23. The compound of item 22, wherein the XRPD pattern further has characteristic peaks expressed in degrees 2θ (±0.2° 2θ) at 14.7, 15.8 and 25.6.

24. The compound of item 22 or 23, wherein the XRPD pattern further has characteristic peaks expressed in degrees 2θ (±0.2° 2θ) at 24.1 and 19.1.

25. The compound of any one of items 22 to 24, wherein the XRPD pattern further has characteristic peaks expressed in degrees 2θ (±0.2° 2θ) at 28.7 and 22.3.

26. The compound of any one of items 1 and 22 to 25, which is crystalline and has a Differential Scanning Calorimetry (DSC) thermogram that exhibits an endotherm having an onset of about 309° C.

27. The compound of item 1, which has an X-ray powder diffraction pattern substantially the same as shown in FIG. 27.

28. A compound of Formula I:

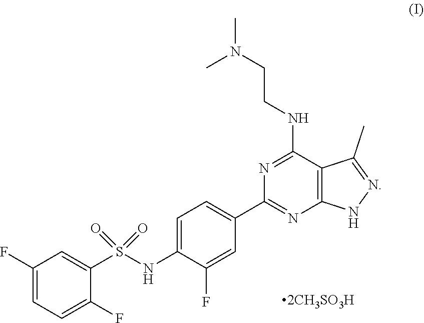 [SMILES: Cc1n[nH]c2nc(-c3ccc(NS(=O)(=O)c4cc(F)ccc4F)c(F)c3)nc(NCCN(C)C)c12 ; "·2CH3SO3H"]

29. The compound of item 28, which is crystalline and exhibits an X-ray powder diffraction (XRPD) pattern having characteristic peaks expressed in degrees 2θ (±0.2° 2θ) at 17.76, 23.38 and 22.80.

30. The compound of item 29, wherein the XRPD pattern further has characteristic peaks expressed in degrees 2θ (±0.2° 2θ) at 12.29, 8.26 and 13.75.

31. The compound of item 29 or 30, wherein the XRPD pattern further has characteristic peaks expressed in degrees 2θ (±0.2° 2θ) at 23.16, 21.63 and 17.07.

32. The compound of any one of items 29 to 31, wherein the XRPD pattern further has characteristic peaks expressed in degrees 2θ (±0.2° 2θ) at 18.44, 15.77 and 10.08.

33. The compound of any one of items 29 to 32, wherein the XRPD pattern further has characteristic peaks expressed in degrees 2θ (±0.2° 2θ) at 24.14, 19.65 and 24.73.

34. The compound of any one of items 28 to 33, which is crystalline and has a Differential Scanning Calorimetry (DSC) thermogram that exhibits an endotherm having an onset of about 164° C.

35. The compound of item 28, which has an X-ray powder diffraction pattern substantially the same as shown in FIG. 12 or 13.

36. The compound of item 28, which has an X-ray powder diffraction pattern substantially the same as shown in FIG. 22.

37. The compound of item 28, which is crystalline and exhibits an X-ray powder diffraction (XRPD) pattern having characteristic peaks expressed in degrees 2θ (±0.2° 2θ) at 22.8, 6.8 and 26.1.

38. The compound of item 37, wherein the XRPD pattern further has characteristic peaks expressed in degrees 2θ (±0.2° 2θ) at 23.7, 15.9 and 18.5.

39. The compound of item 37 or 38, wherein the XRPD pattern further has characteristic peaks expressed in degrees 2θ (±0.2° 2θ) at 17.4 and 22.4.

40. The compound of any one of items 37 to 39, wherein the XRPD pattern further has characteristic peaks expressed in degrees 2θ (±0.2° 2θ) at 31.7 and 27.8.

41. The compound of any one of items 28 and 37 to 40, which is crystalline and has a Differential Scanning Calorimetry (DSC) thermogram that exhibits a first endotherm having an onset of about 74° C. and a second endotherm having an onset of about 218° C.

42. The compound of item 28, which has an X-ray powder diffraction pattern substantially the same as shown in FIG. 37.

43. The compound of item 28, which is crystalline and exhibits an X-ray powder diffraction (XRPD) pattern having characteristic peaks expressed in degrees 2θ (±0.2° 2θ) at 15.2, 16.0 and 17.6.

44. The compound of item 43, wherein the XRPD pattern further has characteristic peaks expressed in degrees 2θ (±0.2° 2θ) at 23.0, 20.5 and 23.3.

45. The compound of item 43 or 44, wherein the XRPD pattern further has characteristic peaks expressed in degrees 2θ (±0.2° 2θ) at 23.9 and 19.3.

46. The compound of any one of items 43 to 45, wherein the XRPD pattern further has characteristic peaks expressed in degrees 2θ (±0.2° 2θ) at 21.1 and 6.9.

47. The compound of any one of items 28 and 43 to 46, which is crystalline and has a Differential Scanning Calorimetry (DSC) thermogram that exhibits a first endotherm having an onset of about 61° C., a second endotherm having an onset of about 140° C. and a third endotherm having an onset of about 218° C.

48. The compound of item 28, which has an X-ray powder diffraction pattern substantially the same as shown in FIG. 39.

49. The compound of item 28, which is crystalline and exhibits an X-ray powder diffraction (XRPD) pattern having characteristic peaks expressed in degrees 2θ (±0.2° 2θ) at 17.6, 23.2 and 22.0.

50. The compound of item 49, wherein the XRPD pattern further has characteristic peaks expressed in degrees 2θ (±0.2° 2θ) at 8.2, 9.8 and 25.9.

51. The compound of item 49 or 50, wherein the XRPD pattern further has characteristic peaks expressed in degrees 2θ (±0.2° 2θ) at 17.1 and 24.1.

52. The compound of any one of items 49 to 51, wherein the XRPD pattern further has characteristic peaks expressed in degrees 2θ (±0.2° 2θ) at 25.2 and 15.0.

53. The compound of any one of items 28 and 49 to 52, which is crystalline and has a Differential Scanning Calorimetry (DSC) thermogram that exhibits a first endotherm having an onset of about 120° C., a second endotherm having an onset of about 165° C. and a third endotherm having an onset of about 215° C.

54. The compound of item 53, wherein the DSC thermogram exhibits an exotherm having an onset of about 180° C.

55. The compound of item 28, which has an X-ray powder diffraction pattern substantially the same as shown in FIG. 41.

56. The compound of item 28, which is crystalline and exhibits an X-ray powder diffraction (XRPD) pattern having characteristic peaks expressed in degrees 2θ (±0.2° 2θ) at 23.4, 12.1 and 24.6.

57. The compound of item 56, wherein the XRPD pattern further has characteristic peaks expressed in degrees 2θ (±0.2° 2θ) at 20.0, 7.9 and 24.9.

58. The compound of item 56 or 57, wherein the XRPD pattern further has characteristic peaks expressed in degrees 2θ (±0.2° 2θ) at 19.0 and 16.6.

59. The compound of any one of items 56 to 58, wherein the XRPD pattern further has characteristic peaks expressed in degrees 2θ (±0.2° 2θ) at 20.6 and 22.1.

60. The compound of any one of items 28 and 56 to 59, which is crystalline and has a Differential Scanning Calorimetry (DSC) thermogram that exhibits a first endotherm having an onset of about 28° C. and a second endotherm having an onset of about 213° C.

61. The compound of item 28, which has an X-ray powder diffraction pattern substantially the same as shown in FIG. 44.

62. A pharmaceutical composition, comprising the compound of any one of items 1 to 61 and a pharmaceutically acceptable carrier or excipient.

63. Use of the compound of any one of items 1 to 61, as an inhibitor of SGK-1.

64. Use of the compound of any one of items 1 to 61, or the pharmaceutical composition of item 19, for the treatment of a cardiovascular disease selected from the group consisting of Long QT syndrome, heart failure, arrhythmia such as atrial fibrillation, ischemic injury, ischemic infarction, cardiac fibrosis, vascular proliferation, restenosis, dilated cardiomyopathy, and stent failure.

65. Use of the compound of any one of items 1 to 61, or the pharmaceutical composition of item 62, for the treatment of Long QT syndrome.

66. The use of item 65, wherein the Long QT syndrome is genetic Long QT syndrome.

67. The use of item 65, wherein the Long QT syndrome is acquired Long QT syndrome.

68. Use of the compound of any one of items 1 to 61, or the pharmaceutical composition of item 62, for the treatment of epilepsy.

69. Use of the compound of any one of items 1 to 61, or the pharmaceutical composition of item 62, for the treatment of Parkinson's disease or Lafora disease.

70. Use of the compound of any one of items 1 to 61 or the pharmaceutical composition of item 62, for the treatment of cancer.

71. The use of item 70, wherein the cancer affects tissues comprising cancerous cells in at least one of the breast, prostate, brain, blood, bone marrow, liver, pancreas, skin, kidney, colon, intestine, endometrium, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, bone, and stomach.

72. The use of item 70, wherein the cancer is a melanoma, liposarcoma, lung cancer, breast cancer, prostate cancer, leukemia, kidney cancer, esophageal cancer, brain cancer, lymphoma, colon cancer or colorectal cancer.

73. The use of item 70, wherein the cancer is prostate cancer, colorectal cancer or breast cancer.

74. Use of the compound of any one of items 1 to 61, for the manufacture of a medicament that inhibits SGK-1 in a subject.

75. Use of the compound of any one of items 1 to 61, for the manufacture of a medicament for the treatment of a cardiovascular disease selected from the group consisting of Long QT syndrome, heart failure, arrhythmia such as atrial fibrillation, ischemic injury, ischemic infarction, cardiac fibrosis, vascular proliferation, restenosis, dilated cardiomyopathy, and stent failure.

76. Use of the compound of any one of items 1 to 61, for the manufacture of a medicament for the treatment of Long QT syndrome.

77. The use of item 76, wherein the Long QT syndrome is genetic Long QT syndrome.

78. The use of item 76, wherein the Long QT syndrome is acquired Long QT syndrome.

79. Use of the compound of any one of items 1 to 61, for the manufacture of a medicament for the treatment of epilepsy.

80. Use of the compound of any one of items 1 to 61, for the manufacture of a medicament for the treatment of parkinson's disease or Lafora disease.

81. Use of the compound of any one of items 1 to 61 for the manufacture of a medicament for the treatment of cancer.

82. The use of item 81, wherein the cancer affects tissues comprising cancerous cells in at least one of the breast, prostate, brain, blood, bone marrow, liver, pancreas, skin, kidney, colon, intestine, endometrium, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, bone, and stomach.

83. The use of item 81, wherein the cancer is a melanoma, liposarcoma, lung cancer, breast cancer, prostate cancer, leukemia, kidney cancer, esophageal cancer, brain cancer, lymphoma, colon cancer or colorectal cancer.

84. The use of item 81, wherein the cancer is prostate cancer, colorectal cancer or breast cancer.

85. A method for inhibiting SGK-1, comprising administering to a subject the compound as defined in any one of items 1 to 61, or the pharmaceutical composition as defined in item 62.

86. A method for the treatment of a cardiovascular disease selected from the group consisting of Long QT syndrome, heart failure, arrhythmia such as atrial fibrillation, ischemic injury, ischemic infarction, cardiac fibrosis, vascular proliferation, restenosis, dilated cardiomyopathy, and stent failure, comprising administering to a subject a therapeutically effective amount of the compound as defined in any one of items 1 to 61, or the pharmaceutical composition as defined in item 62.

87. A method for the treatment of Long QT syndrome, comprising administering to a subject a therapeutically effective amount of the compound as defined in any one of items 1 to 61, or the pharmaceutical composition as defined in item 62.

88. The method of item 87, wherein the Long QT syndrome is genetic Long QT syndrome.

89. The method of item 87, wherein the Long QT syndrome is acquired Long QT syndrome.

90. A method for the treatment of epilepsy, comprising administering to a subject a therapeutically effective amount of the compound as defined in any one of items 1 to 61, or the pharmaceutical composition as defined in item 62.

91. A method for the treatment of parkinson's disease or Lafora disease, comprising administering to a subject a therapeutically effective amount of the compound as defined in any one of items 1 to 61, or the pharmaceutical composition as defined in item 62.

92. A method for the treatment of cancer, comprising administering to a subject a therapeutically effective amount of the compound as defined in any one of items 1 to 61, or the pharmaceutical composition as defined in item 62.

93. The method of item 92, wherein the cancer affects tissues comprising cancerous cells in at least one of the breast, prostate, brain, blood, bone marrow, liver, pancreas, skin, kidney, colon, ovary, lung, testicle, penis, thyroid, parathyroid, pituitary, thymus, retina, uvea, conjunctiva, spleen, head, neck, trachea, gall bladder, rectum, salivary gland, adrenal gland, throat, esophagus, lymph nodes, sweat glands, sebaceous glands, muscle, heart, and stomach.

94. The method of item 93, wherein the cancer is a melanoma, liposarcoma, lung cancer, breast cancer, prostate cancer, leukemia, kidney cancer, esophageal cancer, brain cancer, lymphoma, colon cancer or colorectal cancer.

95. The method of item 94, wherein the cancer is prostate cancer, colorectal cancer or breast cancer.

96. A process for preparing the compound of any one of items 1 to 9, comprising:
dissolving N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide hydrochloride in a solvent to obtain a solution;
filtering the solution;
adding an anti-solvent to the filtrate;
stirring the mixture until a crystalline material is obtained; and isolating the crystalline material.

97. The process of item 96, wherein the solvent is DMSO.

98. The process of item 97, wherein the concentration of N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide hydrochloride in the solvent is between 50 mg/mL and 100 mg/mL.

99. The process of item 97, wherein dissolving is carried out at a temperature of 70° C.

100. The process of any one of items 96 to 99, wherein filtering is carried out at room temperature.

101. The process of any one of items 96 to 99, wherein the anti-solvent is water.

102. The process of item 101, wherein a ratio solvent:anti-solvent is between 2:10 and 0.5:10.

103. The process of item 101, wherein a ratio solvent:anti-solvent is 1:10.

104. The process of any one of items 96 to 103, wherein stirring the mixture is performed at a temperature between 18° C. and 25° C.

105. The process of item 104, wherein stirring the mixture is performed at room temperature.

106. The process of any one of items 96 to 105, wherein stirring the mixture is performed for about 2 to 6 days.

107. The process of item 106, wherein stirring the mixture is performed for about 4 days.

108. The process of any one of items 96 to 107, wherein the crystalline material is dried in a vacuum oven at a drying temperature of 40° C. to 60° C. for about 2 to 4 hours.

109. The process of any one of items 96 to 108, wherein the crystalline material is isolated by filtration.

110. A process for preparing the compound of any one of items 28 to 36, comprising:
combining 1 molar equivalent of N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide with at least 2 molar equivalents of methanesulfonic acid in a solvent;
evaporating at least part of the solvent;
stirring the mixture until a crystalline material is obtained; and
isolating the crystalline material.

111. The process of item 110, wherein the solvent is methanol.

112. The process of item 110, wherein the concentration of N-[4-(4-[[2-(dimethylamino)ethyl]amino]-3-methyl-1H-pyrazolo[3,4-d]pyrimidin-6-yl)-2-fluorophenyl]-2,5-difluorobenzenesulfonamide in the solvent is between 0.1 mol/L and 0.3 mol/L.

113. The process of item 112, wherein the concentration is about 0.2 mol/L.

114. The process of item 111, wherein stirring the mixture is performed at a temperature between 20° C. and 25° C.

115. The process of item 114, wherein the temperature is between 20° C. and 22° C.

116. The process of any one of items 110 to 115, wherein stirring the mixture is performed for about 4 to 6 days.

117. The process of any one of items 110 to 115, wherein stirring the mixture is performed for about 5 days.

118. The process of any one of items 110 to 115, wherein the crystalline material is dried in a vacuum oven at a drying temperature of 62° C. to 72° C. for about one day.

119. The process of any one of items 110 to 118, wherein the crystalline material is isolated by filtration.

Although the invention has been illustrated and described with respect to one or more implementations, equivalent alterations and modifications will occur to others skilled in the art upon the reading and understanding of this specification. In addition, while a particular feature of the invention may have been disclosed with respect to only one of several implementations, such feature may be combined with one or more other features of the other implementations as may be desired and advantageous for any given or particular application.

Accordingly, it is understood that the examples and embodiments described herein are for illustrative purposes only and that various modifications or changes in light thereof will be suggested to persons skilled in the art and are to be included within the spirit and purview of this application and scope of the appended claims. Any publication, document, patent, patent application or publication referred to herein should be construed as incorporated by reference each in their entirety for all purposes.

1-57. (canceled)

58. A compound of Formula II: 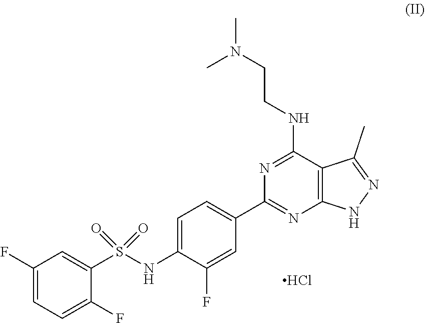 [SMILES: Cc1n[nH]c2nc(-c3ccc(NS(=O)(=O)c4cc(F)ccc4F)c(F)c3)nc(NCCN(C)C)c12 ; Cl]
wherein the compound is crystalline.

59. The compound of claim 58, wherein the compound is an anhydrate.

60. The compound of claim 58, wherein the compound exhibits an X-ray powder diffraction (XRPD) pattern having characteristic peaks expressed in degrees 2θ (±0.2° 2θ) at 6.659 and 22.315.

61. The compound of claim 60, wherein the XRPD pattern further has characteristic peaks expressed in degrees 2θ (±0.2° 2θ) at 15.908 and 20.743.

62. The compound of claim 60, wherein the XRPD pattern further has characteristic peaks expressed in degrees 2θ (±0.2° 2θ) at 28.732 and 20.194.

63. The compound of claim 60, wherein the XRPD pattern further has characteristic peaks expressed in degrees 2θ (±0.2° 2θ) at 24.086 and 25.599.

64. The compound of claim 60, wherein the XRPD pattern further has a characteristic peak expressed in degrees 2θ (±0.2° 2θ) at 19.008.

65. The compound of claim 58, which has a differential scanning calorimetry (DSC) thermogram that exhibits an endotherm having an onset of about 302° C.

66. A pharmaceutical composition, comprising the compound of claim 58 and a pharmaceutically acceptable carrier or excipient.

67. A method for inhibiting SGK-1, the method comprising administering to a subject the compound as defined in claim 58.

68. A method for the treatment of a cardiovascular disease selected from the group consisting of Long QT syndrome, heart failure, arrhythmia such as atrial fibrillation, ischemic injury, ischemic infarction, cardiac fibrosis, vascular proliferation, restenosis, dilated cardiomyopathy, and stent failure, the method comprising administering to a subject a therapeutically effective amount of the compound of claim 58.

69. A method for the treatment of Long QT syndrome, the method comprising administering to a subject a therapeutically effective amount of the compound as defined in claim 58.

70. The method of claim 69, wherein the Long QT syndrome is genetic Long QT syndrome.

71. The method of claim 69, wherein the Long QT syndrome is acquired Long QT syndrome.
